(19) United States
(12) Patent Application Publication
(10) Pub. No.: US 2022/0236268 A1
(43) Pub. Date: Jul. 28, 2022

(54) NOVEL SECRETED ANTIGENS FOR DIAGNOSIS OF ACTIVE BABESIA MICROTI AND BABESIA DUNCANI INFECTION IN HUMANS AND ANIMALs

(71) Applicant: Yale University, New Haven, CT (US)

(72) Inventor: Choukri Ben Mamoun, Farmington, CT (US)

(21) Appl. No.: 17/617,706

(22) PCT Filed: Jun. 12, 2020
(86) PCT No.: PCT/US20/37500

§ 371 Date: Dec. 9, 2021

Related U.S. Application Data
(60) Provisional application No. 62/937,645, filed on Nov. 19, 2019.
(60) Provisional application No. 62/860,662, filed on Jun. 12, 2019.

(51) Int. Cl. G01N 33/569; G01N 33/68

(52) U.S. Cl. CPC G01N 33/56905; G01N 2800/52; G01N 33/6893

(57) ABSTRACT

In various aspects and embodiments, the invention provides a method of detecting a Babesia infection in a subject comprising detecting one or more peptides selected from SEQ ID NOs: 1-62 in a biological sample collected from the subject; wherein detecting one or more of the peptides indicates the presence of Babesia infection.

### CROSS-REFERENCE TO RELATED APPLICATIONS

The present application claims priority under 35 U.S.C. § 119(e) to U.S. Provisional Application No. 62/937,645 filed Nov. 19, 2019, and No. 62/860,662 filed Jun. 12, 2019, all of which applications are hereby incorporated by reference in their entireties herein.

### STATEMENT REGARDING FEDERALLY SPONSORED RESEARCH OR DEVELOPMENT

This invention was made with government support under AI136118, AI123321, AI138139 and GM110506 awarded by National Institutes of Health. The government has certain rights in the invention.

### BACKGROUND OF THE INVENTION

Human babesiosis is an emerging tick-borne disease endemic in the United States and increasingly reported in other parts of the world including Asia and Europe. Three species, Babesia microti, B. duncani and B. divergens have been shown to cause infection in humans with B. microti accounting for the large majority of clinical cases reported worldwide. In susceptible patients, B. microti infection can result in severe symptoms including respiratory distress, splenic rupture and renal failure. The mortality rates associated with babesiosis infections range between 6 and 21%. Furthermore, severe infections as well as end-organ complications may develop in up to 57% of immunocompromised patients. There is a need in the art for compositions and methods useful for the detection and treatment of Babesiosis. The present disclosure addresses this need.

### BRIEF SUMMARY OF THE INVENTION

In certain aspects, the present invention provides methods of detecting a Babesia infection in a subject. In certain embodiments, the method includes: detecting whether one or more secreted antigens selected from SEQ ID NOs: 1-62 are present in a biological sample collected from the subject; wherein detecting presence of one or more of the antigens in the biological sample indicates that the subject has a Babesia infection. In certain embodiments, the method includes: detecting one or more secreted antigens selected from SEQ ID NOs: 1-62 in a biological sample collected from the subject; wherein detecting one or more of the antigens indicates the presence of a Babesia infection. In some embodiments, the Babesia infection comprises Babesia microti or Babesia duncani. In some embodiments, the biological sample comprises one or more selected from the group consisting of: a blood sample, an erythrocyte sample, a leukocyte sample, a plasma sample, a urine sample, a saliva sample, and/or one or more combinations thereof. In some embodiments, the one or more antigens further comprises BmGPI12. In some embodiments, the subject is a human. In some embodiments, the subject is a mammal known to carry a Babesia parasite. In some embodiments, the one or more antigens is detected by one or more antibody-based techniques selected from the group consisting of: Western blot, immunofluorescent assay, IEM, ELISA, PCR amplification-based immunoassay and immunoprecipitation.

In certain aspects the present invention provides a diagnostic tool for identifying or diagnosing a babesiosis infection. In certain embodiments, the tool comprises: an assay platform, an immunologic agent having specificity for one or more Babesia antigens selected from SEQ ID NOs: 1-62. In some embodiments, the babesiosis infection comprises a Babesia microti infection or a Babesia duncani infection. In some embodiments, the assay platform comprises one or more selected from the group consisting of: an enzyme-based assay, a radioimmunoassay, a PCR amplification-based immunoassay, a fluorogenic immunoassay, a chemiluminescence-based assay, immunoblotting assay, and combinations thereof. In some embodiments, the immunologic agent comprises one or more of antibodies or antibody fragments.

In certain aspects, the present invention provides a method of treating, ameliorating, and/or preventing a Babesia infection in a subject in need thereof. In certain embodiments, the method comprises: obtaining a first sample from a subject at a first time point; assaying the sample using a diagnostic tool contemplated herein to detect the presence or absence of an infection relative to a comparator control, administering one or more therapeutic agents to the subject; obtaining a second sample from the subject at a second time point, wherein the second time point comprises one or more time points after the first time point; and assaying the second sample obtained from the subject at the one or more second time points using the diagnostic tool contemplated herein to detect the presence or absence of an infection relative to a comparator control. In some embodiments, the sample comprises a blood sample. In some embodiments, the infection comprises a Babesia microti infection or a Babesia duncani infection. In some embodiments, the first time point is before administration of the therapeutic agent. In some embodiments, the second time point comprises an interval after a therapeutic agent is administered.

In certain embodiments the present invention provides a method of treating, ameliorating, and/or preventing a Babesia infection in a subject. In certain embodiments, the method comprises: detecting presence of one or more peptides selected from SEQ ID NOs: 1-62 in a biological sample collected from the subject; and administering to the subject at least one anti-protozoan therapeutic agent.

### BRIEF DESCRIPTION OF THE DRAWINGS

FIGS. 1A-1B demonstrate that BmGPI12 is secreted into the erythrocyte cytoplasm and subsequently into the extracellular environment of the B. microti-infected erythrocyte. FIG. 1A depicts immunoblotting analysis using pre-immune (PI) and anti-BmGPI12 immune rabbit sera on fractions of uninfected erythrocytes (UI) or erythrocytes infected with B. microti strain LabS1. S: mouse plasma, H: hemolysate; (P) membrane fractions collected following saponin treatment of erythrocytes. In uninfected erythrocytes, the P fraction consists primarily of erythrocyte membrane. In B. microti-infected erythrocytes, the P fraction includes both erythrocyte membrane and protein extracts from isolated parasites. The erythrocyte membrane protein TER-119 (52 kDa) was detected using an anti-TER-119 monoclonal antibody only in the P fractions from uninfected and B. microti-infected red blood cells. FIG. 1B depicts immunofluorescence assay on mouse erythrocytes infected with the LabS1 strain of B. microti. BmGPI12 was labelled with polyclonal anti-BmGPI12 antibodies and could be observed within the parasite cytoplasm, the parasite plasma membrane as well as in the erythrocyte cytoplasm and within individual vesicles (IV) and tubes of vesicles (TOVs) (indicated by arrowheads). Monoclonal antibodies against TER-119 were used to label the plasma membrane of the infected mouse erythrocytes. The DAPI staining was applied to verify the presence of parasites within the erythrocytes by labelling parasitic nuclear DNA. Staining of control uninfected red blood cells is shown. Scale bars: 3 μm.

FIGS. 2A-2C demonstrate that B. microti develops an interlacement of vesicles (IOV) system in the cytoplasm of the infected erythrocytes. FIG. 2A depicts results indicating that giemsa-stained blood smears from B. microti-infected erythrocytes with representative images of LabS1-infected erythrocytes revealed tubular structures within the erythrocyte cytoplasm (see arrowheads). Scale bar: 3 μm. FIGS. 2B and 2C depict analysis of blood smears from four infected mice over a 13-day period following infection with B. microti LabS1 strain. FIG. 2B depicts parasitemia levels in individual mice. A total of 5000 erythrocytes were analyzed per smear (Mean±SEM). FIG. 2C depicts a proportion of each morphological form detected in the blood smears at days 3, 5, 7, 10 and 13 post-infection. A total of 20 images were analyzed per smear at a given day (Mean±SEM).

FIGS. 3A-3C demonstrate that B. microti develops an IOV system in the cytoplasm of the infected erythrocytes. FIG. 3A depicts that EPON-embedded, LabS1-infected erythrocytes revealed the presence of the IOV in the erythrocyte cytoplasm. The IOV contains the same electron dense structures as the parasite, indicating that these structures are of parasitic origin. Various structures of parasites and erythrocytes are shown by arrows. FIGS. 3B and 3C depict a comparison of ultrathin sections of EPON-embedded infected (FIG. 3B) and uninfected (FIG. 3C) erythrocytes. IV: individual vesicle, P: parasite, PPM: parasite plasma membrane, R: ribosomes, RBCC: red blood cell cytoplasm, RBCM: red blood cell membrane, TOV: tube of vesicles.

FIGS. 4A-4D demonstrate that BmGPI12 is localized to the parasite plasma membrane and associated with vesicles and tubules. FIGS. 4A and 4B depict immunoelectron microscopic analysis of B. microti LabS1-infected mouse erythrocytes. Ultrathin sections of high pressure frozen and Durcupan resin-embedded infected erythrocytes were immunolabeled with anti-BmGPI12. IV: individual vesicle, P: parasite, PPM: parasite plasma membrane, RBCC: red blood cell cytoplasm, RBCM: red blood cell membrane, TOV: tube of vesicles. FIG. 4C depicts a schematic diagram showing the steps in the ultracentrifugation of plasma samples collected from B. microti-infected mice. FIG. 4D depicts immunoblot analyses using pre-immune (PI) serum, and anti-BmGPI12 or anti-TER-119 antibodies on either intact plasma (PL) collected from mice infected with B. microti LabS1 strain or on 2 fractions (supernatant: Us, and pellet: Up) of plasma following ultracentrifugation at 120,000×g.

FIGS. 4E-4F depict immunoelectron microscopic analysis of the plasma membrane fraction (Up) from mice infected with B. microti LabS1 using anti-BmGPI12 antibodies coupled to 10 nm gold particles.

FIGS. 5A-5E demonstrate vesicle-mediated secretion of BmIPA48 antigen by B. microti. FIG. 5A depicts distribution of BmIPA48 in the plasma (S), hemolysate (H) and membrane (P) fractions isolated from blood of uninfected or B. microti (LabS1)-infected erythrocytes was determined by western blotting using polyclonal antibodies against BmIPA48 (48 kDa). Pre-immune (PI) sera were used as a control. FIG. 5B depicts immunoblot analysis using pre-immune and anti-BmIPA48 sera on either intact plasma (PL) collected from mice infected with B. microti LabS1 strain or on 2 fractions (supernatant: Us, and pellet: Up) of plasma following ultracentrifugation at 120,000×g. BmIPA48 is associated primarily with the membrane fraction of plasma. FIG. 5C depicts immunofluorescence assay of BmIPA48 distribution in LabS1-infected erythrocytes. BmIPA48 was labelled with polyclonal antibodies and could be detected within the parasite and in discrete IV within the cytoplasm of the infected erythrocyte. Monoclonal antibodies against TER-119 were applied to label the erythrocyte plasma membrane and the DAPI labelling verified the presence of parasitic DNA in the otherwise enucleated host-erythrocytes. Staining of control uninfected red blood cells is shown. FIGS. 5D and 5E depict representative images of immunoelectron micrographs of B. microti LabS1-infected mouse erythrocytes. Ultrathin sections of high pressure frozen and Durcupan resin-embedded infected erythrocytes were immunolabeled with anti-BmIPA48 antibodies coupled to 10 nm gold particles. IV: individual vesicle, P: parasite, PPM: parasite plasma membrane, RBCC: red blood cell cytoplasm, RBCM: red blood cell membrane, TOV: tube of vesicles.

FIGS. 6A-6B depicts a model of vesicular-mediated export of antigens by B. microti.

FIG. 7 demonstrates BmGPI12 distribution in plasma and erythrocytes infected with the PRA99 strain of B. microti. Immunoblotting analysis using pre-immune (PI) and anti-BmGPI12 polyclonal antibodies on fractions of uninfected erythrocytes (UI) or erythrocytes infected with B. microti strain PRA99. S: mouse plasma, H: hemolysate; (P) membrane fractions collected following saponin treatment of erythrocytes. In uninfected erythrocytes, the P fraction consists primarily of erythrocyte membrane. In B. microti-infected erythrocytes, the P fraction includes both erythrocyte membrane and protein extracts from isolated parasites. The erythrocyte membrane protein TER-119 (52 kDa) was detected using an anti-TER-119 monoclonal antibody only in the P fractions from uninfected and B. microti-infected red blood cells.

FIG. 8 demonstrates distribution of the apical end protein BmRON2 in B. microti-infected cells. (A) Immunoblotting analysis using pre-immune (PI) and anti-BmRON2 polyclonal antibodies on fractions of uninfected erythrocytes (UI) or erythrocytes infected with B. microti strain LabS1. S: mouse plasma, H: hemolysate; (P) membrane fractions collected following saponin treatment of erythrocytes. Consistent with previous studies, BmRON2 (163 kDa) undergoes proteolytic degradation in infected cells. The 163 kDa band is found both in the P and S fractions but not in the H fraction consistent with the presence of BmRON2 on the surface of daughter parasites and its release to the plasma following rupture of the infected erythrocyte. No signal was detected when the pre-immune (PI) rabbit serum was used for immunodetection.

FIGS. 9A-9C demonstrate electron microscopy evidence for IOV system emerging from the parasite plasma membrane. Ultrathin EPON-sections of LabS1 B. microti-infected mouse erythrocytes showing individual vesicles (FIG. 9A) as well as tubes of vesicles that are originating from the parasite (FIGS. 9B and 9C). IV: individual vesicle, P: parasite, PPM: parasite plasma membrane, RBCC: red blood cell cytoplasm, RBCM: red blood cell membrane, TOV: tube of vesicles.

FIGS. 10A-10B depict Western blot and immunofluorescence analysis of B. duncani-infected samples and analyzed using antibodies against BdGPI2. FIG. 10A depicts results from a preliminary immunoblot assay using anti-BdGPI2 peptide antibodies (raised in rabbits) on plasma (PL) from uninfected (U) or B. duncani-infected (I) mice (in vivo) or on supernatant (Sup) or parasite fraction (P) from in vitro-cultured B. duncani in human red blood cells (I) or control uninfected human red blood cells (U). FIG. 10B depicts fluorescence microscopy imaging of uninfected or B. duncani-infected human red blood cells using anti-BdGPI2 antibodies. A monoclonal antibody against human Band3 is used as a control to stain the surface of human red blood cells. DAPI is used to stain the nucleus of the parasites

FIGS. 11A-11B depict Western blot and immunofluorescence analysis of B. duncani-infected samples analyzed using antibodies against BdMGF3-1/HSP-70 precurser. FIG. 11A depicts preliminary immunoblot assay using anti-BdHsp70-2 peptide antibodies (raised in rabbits) on plasma (PL) from uninfected (U) or B. duncani-infected (I) mice (in vivo) or on supernatant (Sup) or parasite fraction (P) from in vitro-cultured B. duncani in human red blood cells (I) or control uninfected human red blood cells (U). FIG. 11B depicts fluorescence microscopy on uninfected or B. duncani-infected human red blood cells using anti-BdHsp70-2 antibodies. A monoclonal antibody against human Band3 is used as a control to stain the surface of human red blood cells. DAPI is used to stain the nucleus of the parasites.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, to “alleviate” a disease, defect, disorder or condition means reducing the severity of one or more symptoms of the disease, defect, disorder or condition.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

The term “immune response” as used herein is defined as a cellular response to an antigen that occurs when lymphocytes identify antigenic molecules as foreign and induce the formation of antibodies and/or activate lymphocytes to remove the antigen.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human.

As used herein, to “treat” means reducing the frequency with which symptoms of a disease, defect, disorder, or adverse condition, and the like, are experienced by a patient.

The term “therapeutic” as used herein means a treatment and/or prophylaxis. A therapeutic effect is obtained by suppression, remission, or eradication of a disease state.

As used herein, a “therapeutically effective amount” is the amount of a composition of the invention sufficient to provide a beneficial effect to the individual to whom the composition is administered.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for detecting, diagnosing, screening for, treating and/or reducing Babesia infections such as babesiosis in a subject. The Babesia infection may include Babesia microti (B. microti) infections, Babesia duncani (B. duncani) infections, and/or combinations thereof. The subject may include a mammal. In certain embodiments, the subject is a human.

Compositions and Methods for Detecting and/or Diagnosing an Infection

In certain embodiments, the present invention provides compositions and methods for detecting an infection in a subject. The infection includes parasitic infections such as Babesia infections caused by one or more Babesia strains. The Babesia infection may include Babesia microti (B. microti) infections, Babesia duncani (B. duncani) infections, and/or combinations thereof.

In certain embodiments, the compositions includes one or more compositions for detecting one or more secreted antigens. The one or more antigens include one or more antigens generated by one or more cells infected with a Babesia infection (e.g. B. duncani infection, B. microti infection and/or combination thereof). The one or more antigens may include one or more peptides including one or more of SEQ ID NOs: 1-62.

### Compositions for Detecting/Diagnosing a Babesia Infection

In certain aspects, the present invention provides compositions for detecting and/or diagnosing and infection in a subject. The compositions may include one or more immunologic agents for detecting one or more antigens or peptides generated by one or more cells. The one or more antigens or peptides may be detected in a sample obtained from a subject with an infection.

In various embodiments, the one or more immunologic agents include one or more immunologic agents that have specificity for one or more antigens generated by the one or more cells. For example the one or more immunologic agents may include one or more of an antibody, an antibody fragment, antisense peptide, synthetic peptide, synthetic antibody, synthetic antibody fragment, synthetic hybridized antibody or antibody fragment. The one or more immunologic agents detect, bind to, or interact with one or more peptides or antigens generated by one or more cells infected with one or more of B. duncani, B. microti, or both. The one or more peptides of antigens may include one or more selected from SEQ. ID. NOs: 1-46, 47-62. In some embodiments, one or more antigens or peptides selected from SEQ. ID. NOs: 1-46 may be used to detect a B. duncani infection in a sample obtained from a subject. In some embodiments, one or more antigens or peptides selected from SEQ. ID. NOs: 47-62 may be used to detect a B. microti infection in a sample obtained from a subject.

In certain embodiments, the biological sample includes one or more of blood, urine, saliva, feces, lymph, bile and the like, and/or one or more combinations thereof. The sample may include one or more of a whole blood sample, a plasma sample, a serum sample, a hemolysate sample, and the like. In some embodiments, the one or more infected cells include one or more blood-derived cells such as one or more of erythrocyte, leukocyte, plasma cell, platelets, and the like.

Methods for Detecting/Diagnosing a Babesia Infection

In certain embodiments, the present invention relates to methods for detecting and/or diagnosing one or more Babesia infections including babesiosis, B. duncani infection, B. microti infection, and/or combinations thereof.

In certain embodiments, the methods include obtaining one or more biological samples obtained from a subject. The one or more biological samples may include one or more of blood, urine, saliva, feces, lymph, bile and the like, and/or one or more combinations thereof. The blood sample may include whole blood sample, a plasma sample, a serum sample, a hemolysate sample, and the like. The subject may include a human subject. The subject may have a known or suspected infection of one or more Babesia strains including one or more of B. duncani, B. microti, and/or combinations thereof.

Embodiments of the methods include detecting one or more infections in one or more biological samples obtained from a subject. The one or more infections include one or more of babesiosis, B. duncani infection, B. microti infection, and/or combinations thereof. The infection may be detected using one or more immunologic agent having specificity for one or more antigens or peptides including one or more of SEQ. ID. NOs: 1-46, 47-62, and/or one or more combinations thereof. In certain embodiments, the one or more antigens or peptides are detected using one or more assay platforms including for example, an enzyme-based assay, a radioimmunoassay, a PCR amplification-based assay, a fluorogenic immunoassay, a chemiluminescence-based assay, immunoblotting assay, and combinations thereof. The assay may include one or more of Western blot, immunofluorescence, immune-electron microscopy, ELISA, immunoprecipitation, and the like.

In certain embodiments, the one or more antigens or peptides are detected relative to a comparator control. In certain embodiments, an infection is detected if the signal is greater than or less than a threshold value, a difference relative to a comparator control, or the like.

Embodiments of the methods include measuring one or more biological samples obtained from a subject in order to detect the presence of an infection. Embodiments of the methods include measuring one or more biological samples obtained from a subject in order to evaluate the efficacy of one or more therapeutic agents. In various embodiments, the sample is a blood sample, a sera sample, and/or one or more samples containing one or more other suitable bodily fluids. In various embodiments, the subject is a subject that is healthy, a subject that is confirmed to be infected, a subject that is suspected to be infected, a subject that has been treated for an infection and is believed to no longer be infected, and the like. The subject may be evaluated by assaying the one or more samples obtained from the subject. The one or more samples may be evaluated at one or more time points in order to evaluate the status of an infection and/or the efficacy of one or more treatments for the infection. In various embodiments, the one or more time points include prior to an intentional infection, such as about 24 hours prior to infection, from about 24 hours to about 12 hours prior to infection, from about 12 hours to about 8 hours prior to infection, from about 8 hours to about 4 hours prior to infection, from about 4 hours to about 1 hour prior to infection, less than about 1 hour prior to infection, and any and all intervals and increments therebetween. The one or more time points include at the time of infection, up to 1 hour post-infection, from about 1 hour to about 1 day post-infection, from about 1 day to about 2 days post infection, about 2 days to about 3 days post-infection, from about 3 days to about 4 days post-infection, from about 4 days to about 5 day post-infection, from about 5 days to about 6 days post-infection, from about 6 day to about 7 days post-infection, from about 1 week to about 2 weeks post-infection, from about 2 weeks to about 4 weeks post-infection, from about 4 weeks to about 6 weeks post-infection, and so on. In various embodiments, the one or more time points include at the time of infection, immediately prior to administering a therapeutic agents, at the time of administering a therapeutic agent, and at one or more time points after administration of a therapeutic agent. The one or more time points may include up to 24 hours prior to administration of a therapeutic agent, from about 24 hours to about 12 hours prior to administration of a therapeutic agent, from about 12 hours to about 8 hours prior to administration of a therapeutic agent, from about 8 hours to about 4 hours prior to administration of a therapeutic agent, from about 4 hours to about 1 hour prior to administration of a therapeutic agent, less than about 1 hour prior to administration of a therapeutic agent, and any and all increments and intervals therebetween. In various embodiments, the one or more time points include: up to 1 hour post-administration, from about 1 hour to about 12 hours post-administration, from about 12 hours to about 24 hours post-administration, from about 1 day to about 2 days post-administration, from about 2 day to about 5 days post-administration, from about 5 days to about 7 days post-administration, from about 7 days to about 14 days post-administration, from about 14 day to about 28 days post-administration, from about 28 days to about 42 days post-administration, and so on. The one or more time points may include from about 1 week to about 2 weeks post-administration, from about 2 weeks to about 4 weeks post-administration, from about 4 weeks to about 6 weeks post-administration, and so on.

The methods include detecting an infection in a subject using one or more techniques as described herein. The methods include administering to the subject an effective amount of at least one therapeutic agent including one or more anti-protazoan therapeutic agents. In various embodiments, the anti-protazoan therapeutic agent includes one or more anti-protazoan agents as understood in the art.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

The apicomplexan parasite Babesia microti is the primary agent of human babesiosis, a malaria-like illness and potentially fatal tick-borne disease. Unlike its close relatives, the agents of human malaria, B. microti develops within human and mouse red blood cells in the absence of a parasitophorous vacuole, and its secreted antigens lack trafficking motifs found in malarial secreted antigens. Here, it is shows that after invasion of erythrocytes, B. microti undergoes a major morphogenic change during which it produces an interlacement of vesicles (IOV); the IOV system extends from the plasma membrane of the parasite into the cytoplasm of the host erythrocyte.

### Methods

Parasite Strains

B. microti isolates used in this study are LabS1 and PRA99. These strains were maintained in rag2−/− Knockout (B6.12956-Rag2tmlFwa N12) and SCID (severe combined immunodeficiency) C.B17 SCID C.B-Igh-1b/IcrTac-Prkdcscid and CB17/Icr-Prkdcscid/IcrIcoCrl mice as previously described. Parasitemia was determined by thin blood recombinant protein consisting of amino acids 1-302 of BmGPI12, BmIPA48 (Genebank ID # XP_021338473; EupathDB ID #: BMR1_03g00947) peptide CNKIKTDGGKVDSNS, BmRON2 peptide NKIKTDGGKVDSNS. Monoclonal anti-mouse TER-119 (INVITROGEN®) was used as a control.

Plasma Collection and Fractionation Studies

Blood from uninfected or B. microti-infected animals was collected by cardiac puncture and stored in tubes containing K₂EDTA (dipotassium ethylenediaminetetraacetate) solution. For plasma separation, samples were spun down at 1,300 rpm (200×g) for 20 minutes at room temperature. Plasma or supernatant was removed and added to new 1.7 ml microcentrifuge tubes. The remaining cell pellet was washed twice with PBS supplemented with 1% saponin, incubated on ice for 30 minutes and spun at 9,300×g for 10 minutes at 4° C. The resulting supernatant (hemolysate) was collected and the remaining pellet (uninfected) or parasite (infected) fractions were washed twice with PBS and spun at 9,300×g for 10 minutes at 4° C. Plasma (S), hemolysate (H) and pellet (P) fractions were mixed with Lammeli buffer, separated on SDS-PAGE and analyzed by immunoblotting.

Isolation of Vesicles from Plasma

IOVs were isolated from the plasma of uninfected mice or mice infected with B. microti by sequential centrifugations following the protocol for the isolation of exosomes with some modifications. Briefly, 400 μm plasma from animals was diluted with 5 ml PBS and centrifuged at 500×g for 30 minutes and then at 16,000×g for 45 min to remove microvesicles. IOVs were pelleted with ultracentrifugation (UC) at 120,000×g for 14 hours at 4° C. using a SORVALL™ MTX 150 Micro-Ultracentrifuge with a S52-ST Swinging-Bucket Rotor (THERMO FISHER SCIENTIFIC). The resulting pellet (P1) was collected and the supernatant was spun again using the same conditions. The resulting pellet (P2) and supernatant (Us) fractions were collected.

Immunoblot Analysis

Equal concentrations of plasma (prior to ultracentrifugation), supernatant (Us) and pellet (Up) fractions obtained after UC were analyzed by immunoblotting. To obtain equal concentrations, supernatant (Us) fractions were concentrated with 20% trichloroacetic acid. The pellet fractions (P1 and P2) were diluted further and combined as a single pellet (Up). All the samples were resuspended in Lammeli-buffer and loaded on 10% Mini-PROTEAN® TGX (BIO-RAD LABORATORIES®, Hercules, Calif.) and transferred to nitrocellulose membranes. Membranes were blocked in 5% milk and incubated with a rabbit anti-BmGPI12 serum (1:250) or pre-immune serum (1:250 dilution) overnight at 4° C. The next day, membranes were washed in TBS-T and incubated with ECL-horseradish peroxidase (HRP)-conjugated anti-rabbit IgG (1:10,000 dilution) for 1 hour. Following additional washes, the membranes were incubated with ECL western blotting detection reagents (GE HEALTHCARE®, Amersham, UK) and exposed to X-ray film using KODAK® autoradiography. The same method was used for immunoblotting of parasite and erythrocyte membrane (P), erythrocyte cytoplasm or hemolysate (H) and plasma or supernatant (S) fractions. Polyclonal antibodies including rabbit anti-BmIPA48 (1:100) and rabbit anti-BmRON2 (1:100) and their corresponding pre-immune sera and monoclonal anti-mouse TER-119 antibody (1:500) were also analyzed by immunoblotting.

Immunofluorescence Assay (IFA)

Rabbit anti-BmGPI12 (BmSA-1) antibodies were used to assess the localization of BmGPI12 in B. microti LabS1 and PRA99. Rabbit anti-BmIPA48 serum was used to evaluate the localization of BmIPA48 in LabS1. Blood smears were prepared from retro orbital bleeding (after centrifugation at 1,500 rpm for 2 min (MINISPIN PLUS®, EPPENDORF™) on thin 22 mm×22 mm microscopy cover glasses (12-542-B; FISHERBRAND®) and immediately transferred into a 6-well plate (COSTAR®, CORNING®, Inc.) which was put on a cold metal plate on ice. Afterwards 6-well plates with the blood smears were stored at 4° C. before being further processed. To perform the IFA, thin blood smears were removed from the 4° C. storage and fixed in 1% formaldehyde (28908; THERMO SCIENTIFIC®) diluted in PBS (10010-023; GIBCO®) at 37° C. (PRECISION® mechanical convection incubator, model 6 LM) for 30 minutes followed by three brief rinses in PBS. Smears were then incubated for 1 hour in a blocking buffer (5% heat-inactivated fetal bovine serum (16000-044, GIBCO®) 5% normal goat serum (16210-072; GIBCO-BRL®) and 0.1% saponin (10% stock solution in PBS) (57900-100G; SIGMA®)) at 37° C. and then once rinsed in wash buffer (0.5% fetal bovine serum, 0.5% normal goat serum, 0.05% saponin). To target the mouse erythrocyte membrane, FITC anti-mouse TER-119/Erythroid Cells; Clone: TER-119 (116206; BIOLEGEND®) was used at a 1:1,000 dilution. The anti-BmGPI12 or anti-BmIPA48 antibodies were used at 1:1,000 dilution.

The smears were either incubated overnight at 4° C. while being rocked gently (Orbi-Blotter; BENCHMARK®) or for 1 hour at 37° C. The next day, slides were washed three times for 2 minutes each with wash buffer and incubated with secondary antibody, Goat anti-rabbit IgG (H+L) rhodamine conjugate (31670, Invitrogen) (1:1,000) at 37° C. for 1 hour and washed three times for 2 minutes each in wash buffer. After three brief rinses in PBS and one brief rinse in ddH₂O, the coverslips were mounted on sandblasted single frosted pre-cleaned microscope slides (421-004T; THERMO SCIENTIFIC®) using PROLONG™ Gold antifade reagent supplemented with DAPI (P36935; INVITROGEN® by THERMO FISHER SCIENTIFIC®) and incubated at RT in the dark overnight, before they were examined with the LEICA® TCS SP8 STED 3× microscope (LEICA® Microsystems GmbH; Wetzlar, Germany).

Imaging on Leica TCS SP8 STED 3×

The confocal images were acquired with a LEICA® TCS SP8 STED 3× microscope. A HC PL APO CS2 100×/1.40 oil immersion objective was used for image acquisition. FITC anti-mouse TER-119/Erythroid Cells; Clone: TER-119 was excited at 488 nm (500 nm-571 nm, HyD3), rhodamine was excited at 550 nm (569 nm-650 nm, HyD3) and DAPI was excited at 405 nm (430 nm-470 nm, HyD1). The pinhole was set to 1 AU. The images were acquired in unidirectional confocal mode with 1,000 Hz scan speed (line average 6). The image size was image size was chosen to be 38.75 μm×38.75 μm (1,024×1,024 pixels). The PMT Trans was further activated to enable the acquisition of DIC images.

Sample Preparation for Electron Microscopy Cryosectioning

The sample pellet was fixed in 4% paraformaldehyde (PFA) in PBS for 30 minutes at room temperature followed by further fixation in 4% PFA at 4° C. for 1 hour. They were rinsed in PBS and re-suspended in 10% gelatin. Chilled blocks were trimmed and placed in 2.3 M sucrose overnight on a rotor at 4° C. They were transferred to aluminum pins and frozen rapidly in liquid nitrogen. The frozen blocks were cut on a LEICA® Cryo-EMUC6 UltraCut and 65 nm thick sections were collected using the Tokoyasu method (Tokuyasu, 1973) and placed on carbon/formvar-coated grids and floated in a dish of PBS for immunolabeling.

Immunolabeling of the Ultra-Thin Sections

Samples were processed according to the method described by Slot and Geuze (Slot and Geuze, Nature Protocols. 2007; 2(10):2480-2491.). The grids were incubated on rabbit anti-BmGPI12 (1:100) or rabbit anti-BmIPA48 (1:100) and their corresponding pre-immune sera (1:500). For secondary antibody, 10 nm Protein A gold (Utrecht Medical Center) was used. All grids were rinsed in PBS, fixed using 1% glutaraldehyde for 5 minutes, rinsed again and transferred to a UA/methylcellulose drop before being collected and dried.

High Pressure Freezing and Freeze Substitution Epon Section and Labeling

Samples fixed in 4% PFA were frozen using a LEICA® HMP100 at 2000 psi. The frozen samples were then freeze substituted using a LEICA® Freeze AFS unit starting at −95° C. using 1% osmium tetroxide, 1% glutaraldehyde and 1% water in acetone for 10 h, warmed to −20° C. for 12 hours and then to 4° C. for 2 hours. The samples were well rinsed in 100% acetone and infiltrated with DURCUPAN™ resin (ELECTRON MICROSCOPY SCIENCES®) and baked at 60° C. for 24 hours. Hardened blocks were cut using a LEICA® UltraCut UC7 and 60 nm sections were collected on formvar/carbon coated nickel grids.

Immuno-Labeling of Resin Sections Grids were placed section side down on drops of 1% hydrogen peroxide for 5 minutes, rinsed and blocked for non-specific binding on 3% bovine serum albumin in PBS containing 1% Triton-X for 30 minutes. Grids were incubated with a primary antibody rabbit anti-BmGPI12 or anti-BmIPA48 1:100 overnight, rinsed in buffer and then incubated with the secondary antibody 10 nm protein A gold (UtrechtUMC) for 30 minutes. The grids were rinsed and fixed using 1% glutaraldehyde for 5 minutes, rinsed well in distilled water, and contrast stained using 2% uranyl acetate and lead citrate. Grids were all viewed in a FEI Tencai Biotwin TEM at 80 kV. Images were taken using Morada CCD and iTEM (OLYMPUS®) software.

Image Processing

FIJI (imagej.net/Fiji) and MICROSOFT® POWERPOINT® (MICROSCOFT® Corporation) was used to analyze and prepare raw images of Giemsa smears, EM images and fluorescence images for presentation. Diameter and length of EM structures were determined via the line tool in FIJI. The Huygens Professional Software (Scientific Volume Imaging) was further used to deconvolve the fluorescence images acquired with the LEICA® TCS SP8 STED 3×.

### Selected Results

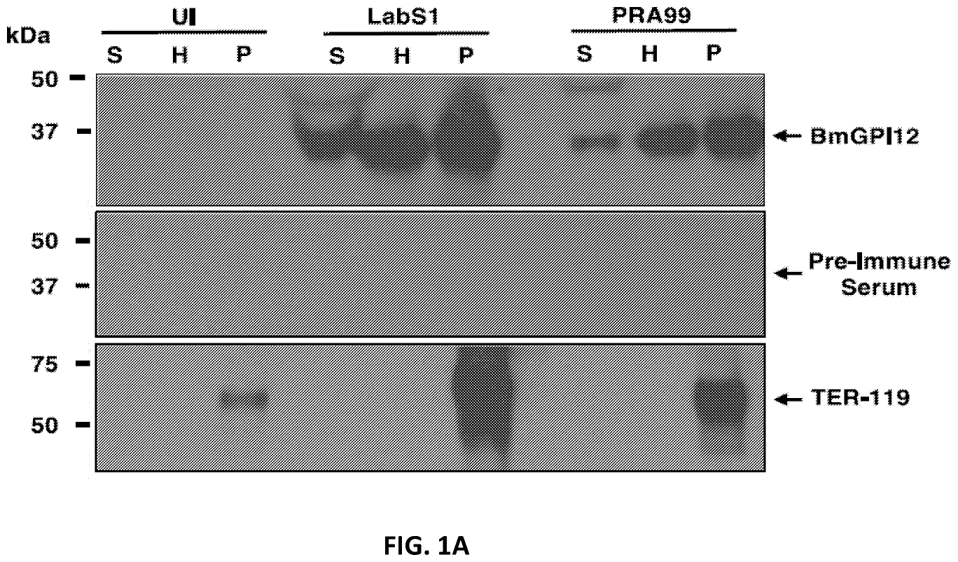

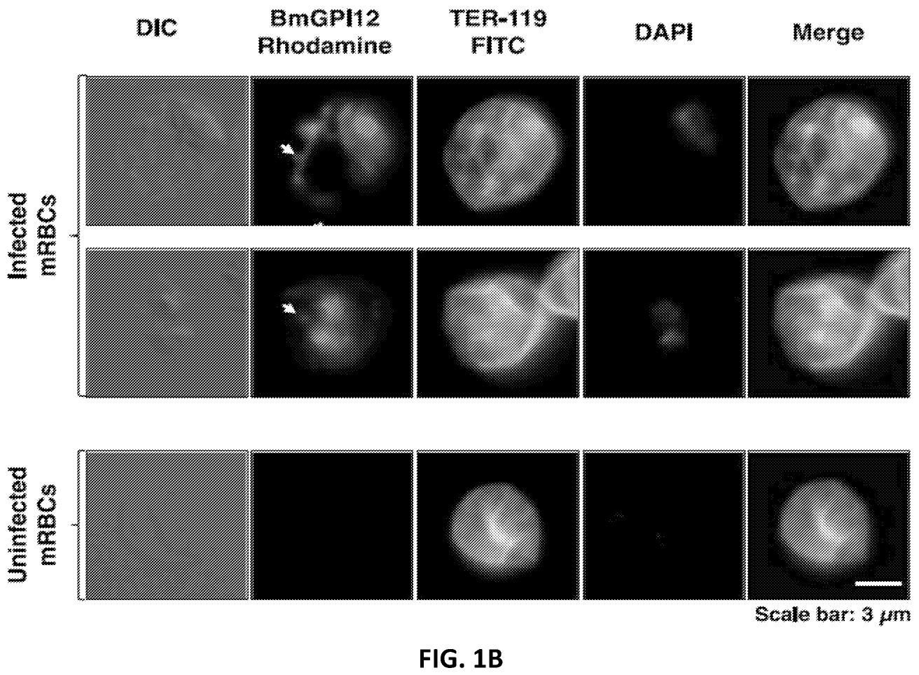

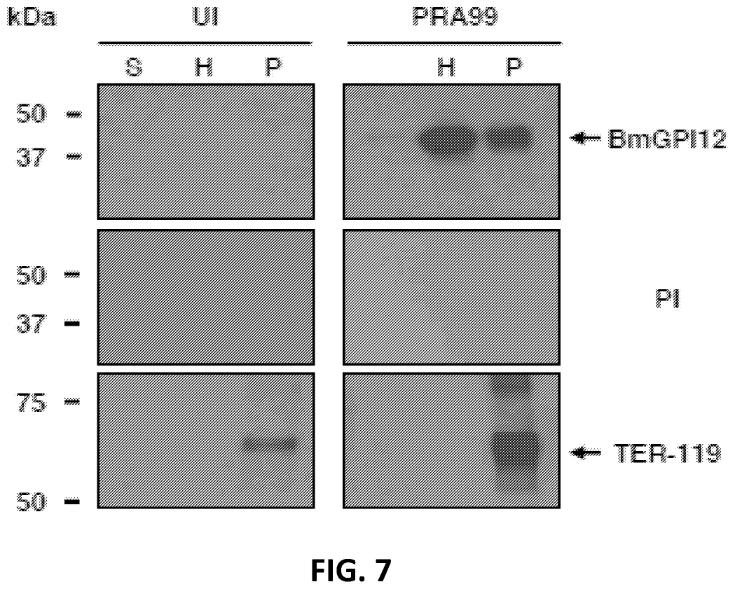

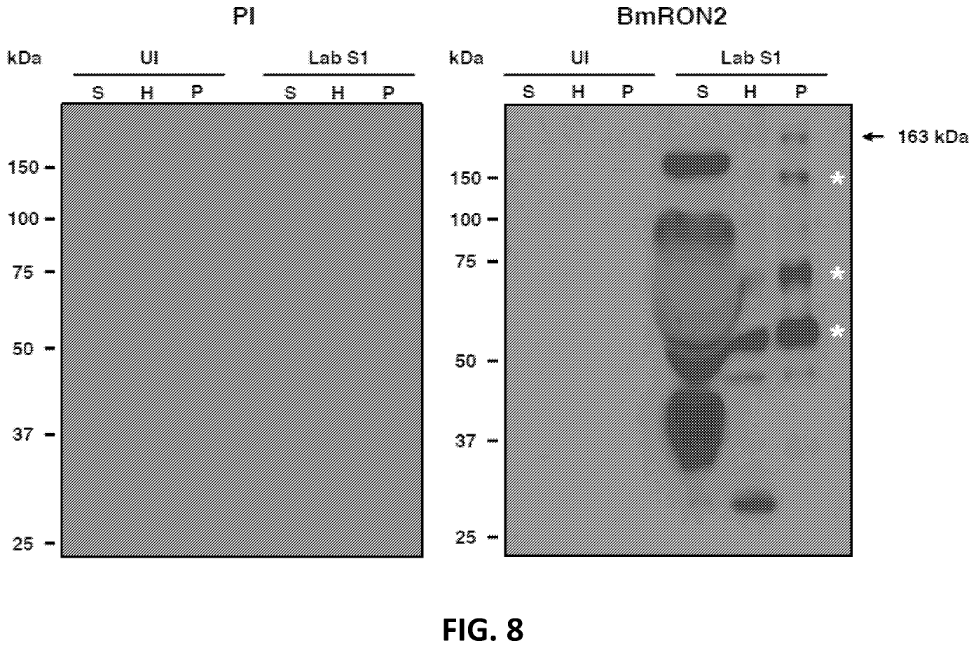

B. microti major secreted antigen localizes to vesicular structures associated with parasite morphogenesis. The immunodominant BmGPI12 of B. microti is encoded by a member of the bmn multigene family and one of the most highly expressed genes of the parasite during its development within red blood cells. Consistent with the secretion of BmGPI12 from the parasite into the red blood cytoplasm and subsequently into the host environment, immunoblot analyses using anti-BmGPI12 antibodies on blood collected from mice and fractionated to collect plasma (S), erythrocyte cytoplasm (H), and membrane (P) fractions showed the presence of BmGPI12 in all three fractions from animals infected with B. microti strains (LabS1 (FIG. 1) or PRA99 (FIG. 7)) but not from uninfected animals (FIG. 1A and FIG. 7). As a control, immunoblot analyses conducted using a monoclonal antibody against the mouse erythrocyte membrane protein, TER-119 (glycophorin A-associated protein (Ly-76)) identified this protein in the membrane (P) fractions of both uninfected and B. microti-infected erythrocytes (Kina et al., 2000, Br J Haematol. 109:280-287) but not in the plasma or erythrocyte cytoplasm fractions (FIG. 1B). As expected, antibodies against the B. microti rhoptry neck protein BmRON2, a highly conserved protein among apicomplexan parasites that localizes to the parasite apical end (Ord et al., 2016, Infect Immun. 84:1574-1584), identified the protein in the membrane and plasma fractions, but not in the erythrocyte cytoplasm (FIG. 8). This finding is consistent with the association of BmRON2 with the parasite during its intra-erythrocytic development and its release following the rupture of the infected erythrocyte and exit of daughter parasites (Ord et al., 2016, Infect Immun. 84:1574-1584). As a control, pre-immune sera were used to analyze the fractions from uninfected and B. microti-infected erythrocytes and no signal could be detected (FIGS. 7 and 8).

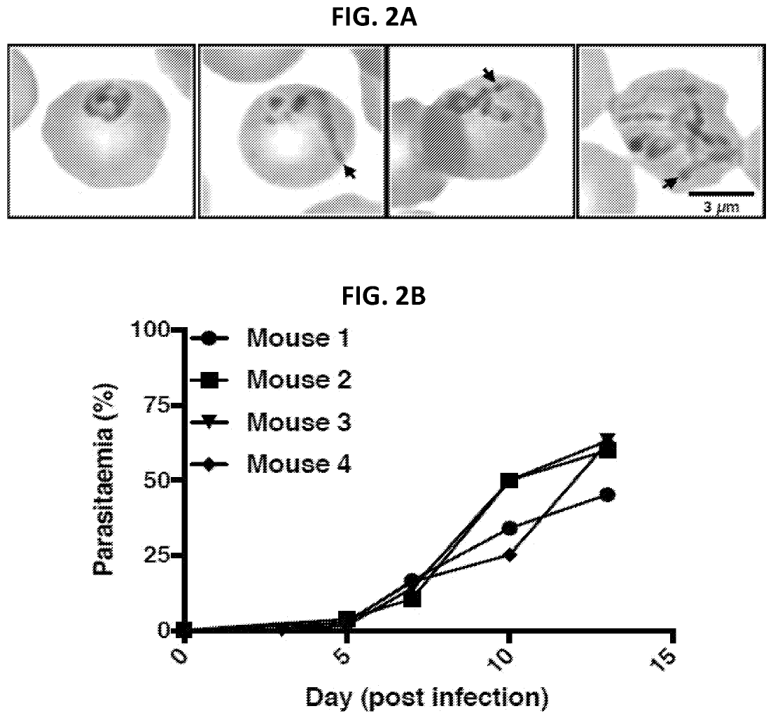

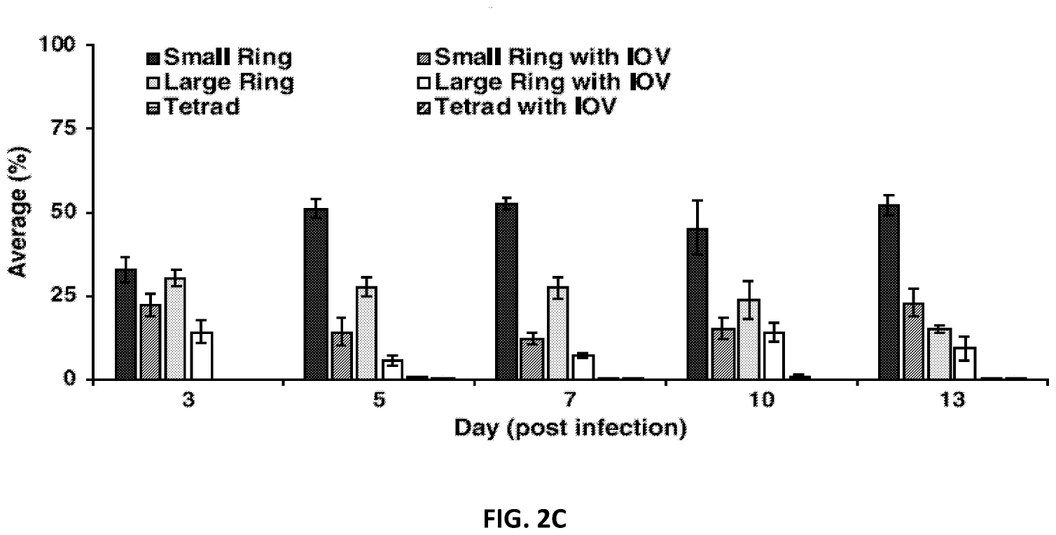

The localization of BmGPI12 was further examined by confocal microscopy. The analysis identified BmGPI12 in both the cytoplasm and plasma membrane of the parasite as well as in the cytoplasm of the infected erythrocyte in well-defined dendrite-like structures and distinct foci (FIG. 1B). These structures are reminiscent of membranous extensions often seen in Giemsa-stained blood smears of B. microti-infected erythrocytes at different stages of parasite development (representative images of LabS1) (FIG. 2A). Blood smears prepared from four B. microti-infected mice showed that the parasite undergoes major morphogenic changes throughout its intraerythrocytic life cycle that include ring-shaped forms, rings with dendrite-like tubovesicular structures (TOVs), dividing parasites (tetrads) and tetrads with TOVs (FIGS. 2B and 2C). The proportion of parasites with TOV structures was similar as parasite burden increased in infected animals suggesting that the morphogenic events are part of the intraerythrocytic life cycle of B. microti (FIG. 2C).

Vesicle-Mediated Export of BmGPI12 Antigen into the Cytoplasm of B. microti-Erythrocytes and Host Plasma

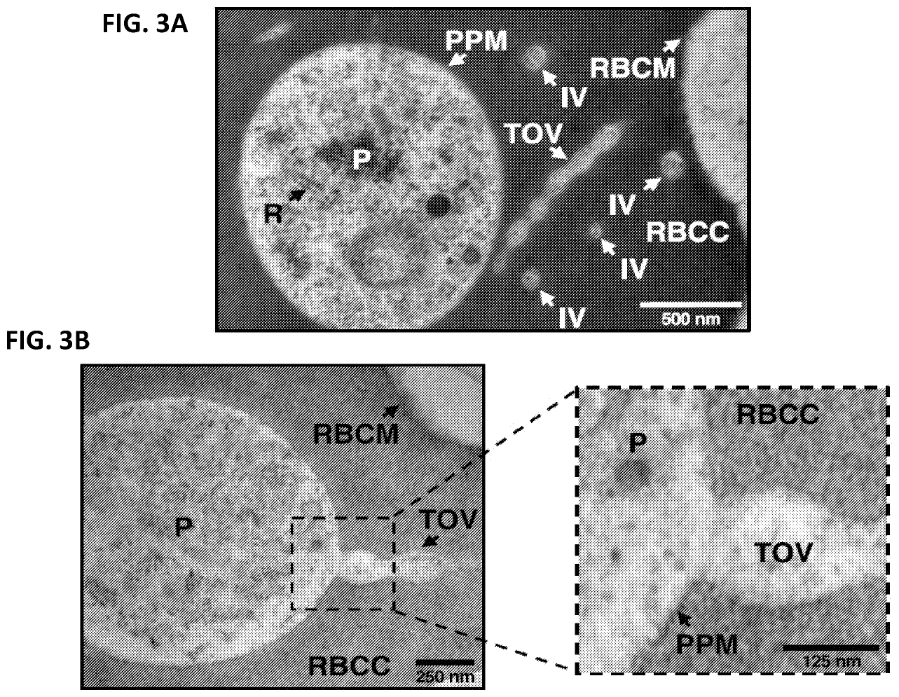

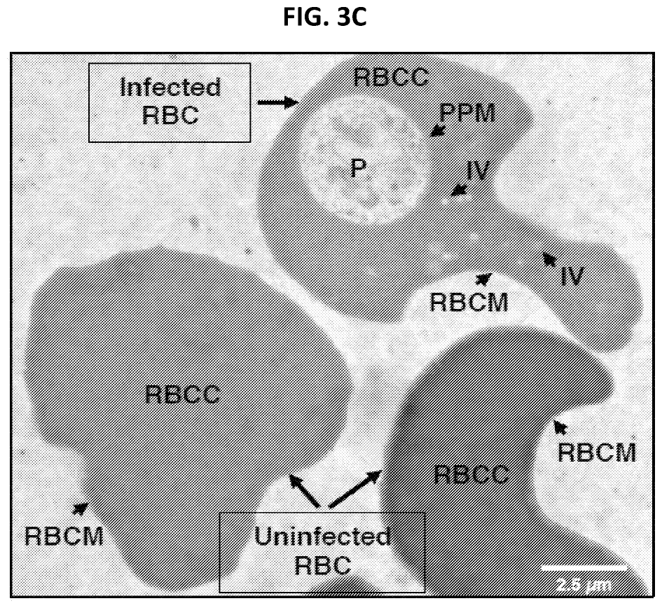

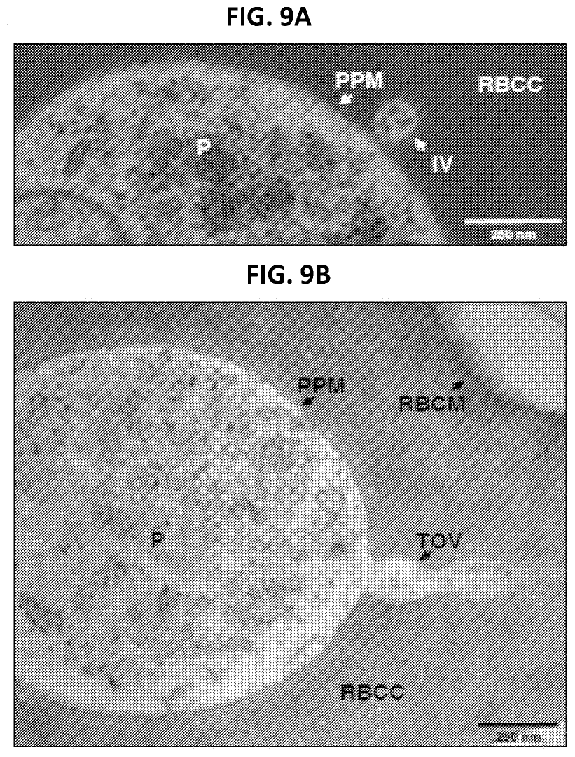

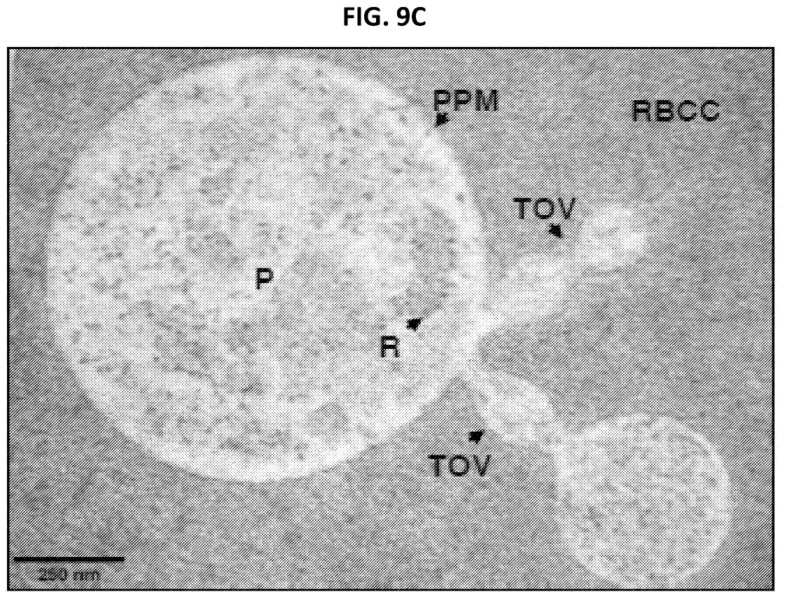

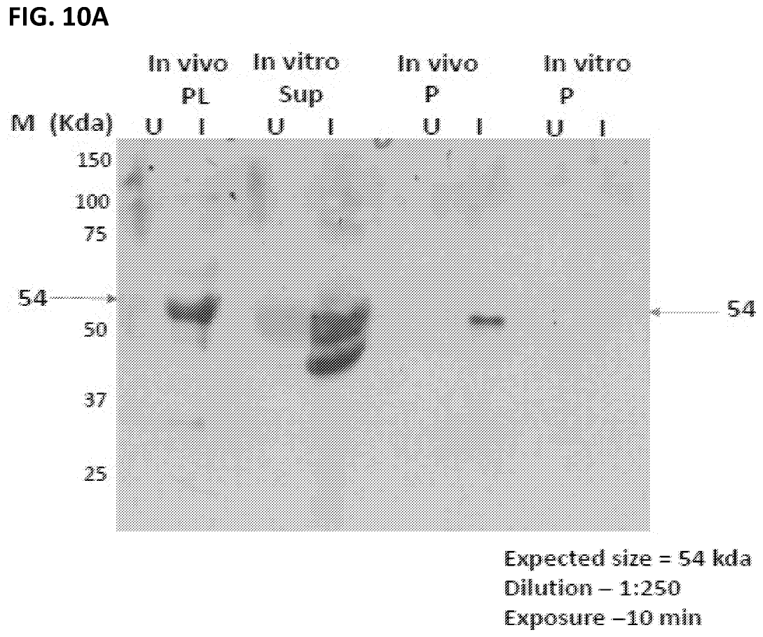

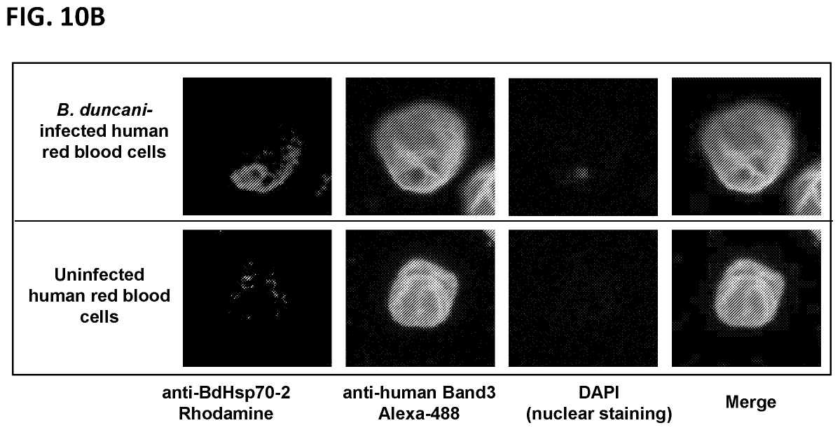

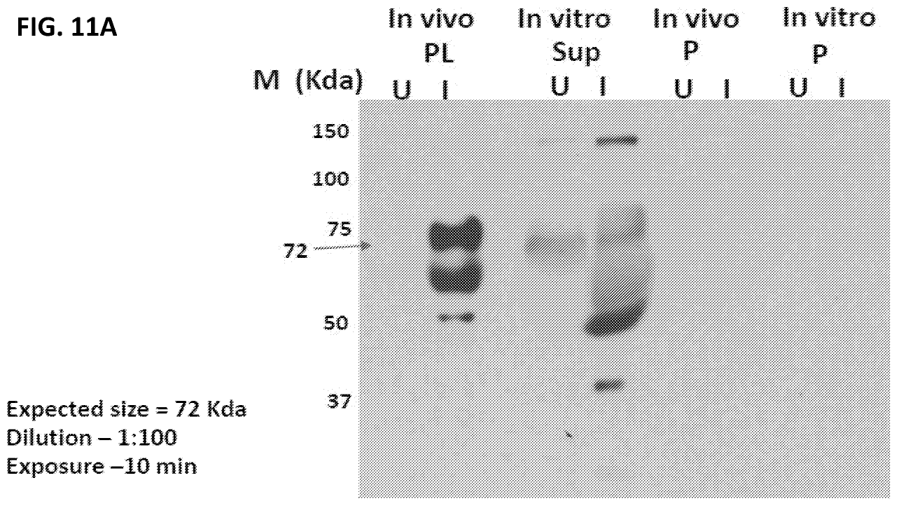

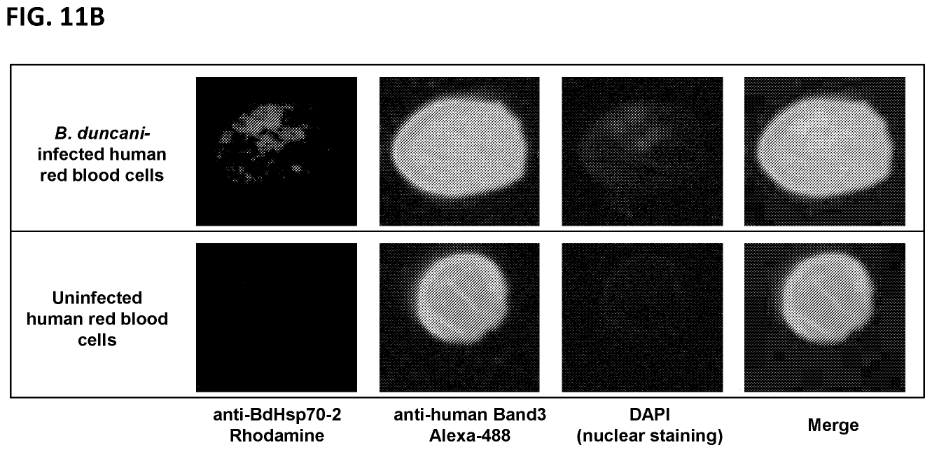

To investigate at the ultrastructural level the nature of the TOVs, electron microscopy analyses of ultrathin sections of erythrocytes from B. microti-infected mice were conducted. These analyses revealed tubes of connected vesicles as well as individual vesicles in the cytoplasm of infected cells (FIGS. 3A and 3B, and FIG. 9), but not in the uninfected cells (FIG. 3C). The measured diameter of individual vesicles (IV) is approximately 0.110 μm±0.0052 μm (Mean±SEM), whereas the length of tubes of vesicles (TOV) ranges between 0.405 μm±0.056 μm (Mean±SEM)) and 0.900 μm, depending on the sections. Interestingly, detailed analysis of these structures revealed that the TOVs emerge directly from the parasite plasma membrane and extend into the infected erythrocyte (FIG. 3B and FIG. 9 (panels B and C)). Both the cytoplasm of the parasite and the content of the vesicles and tubules share the same electron density (FIGS. 3A and 3B, and FIG. 7), further demonstrating that the structures are of parasite origin.

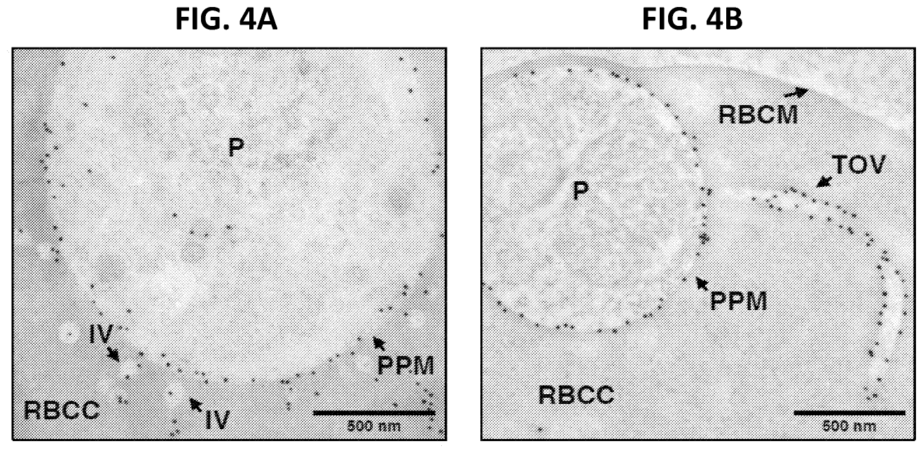

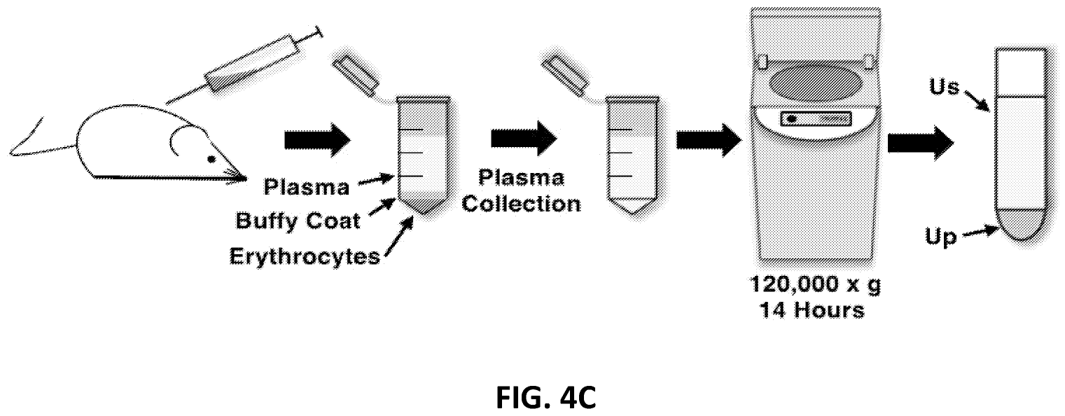

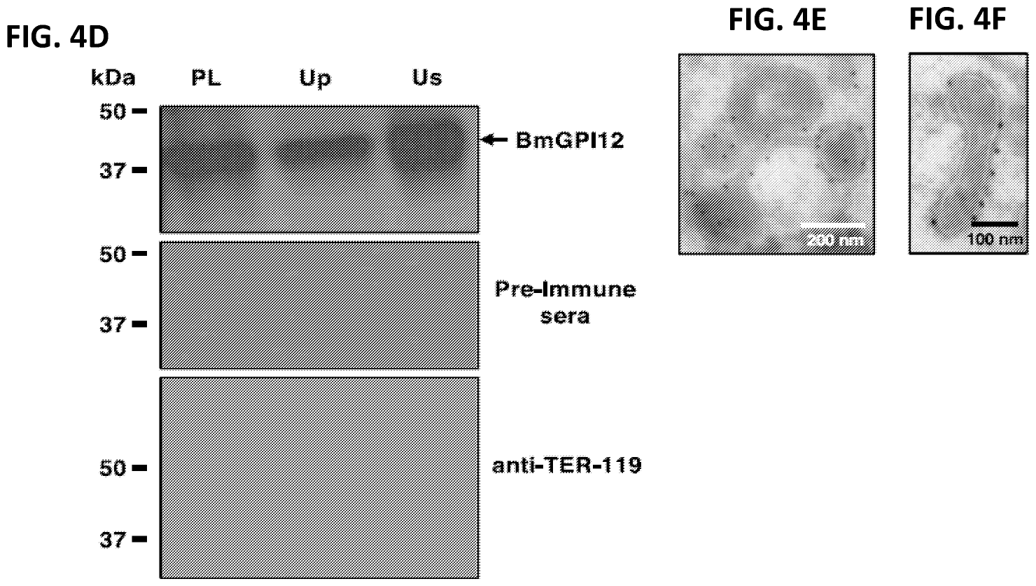

To determine whether the IV and TOV structures identified by electron microscopy are the same BmGPI12-positive structures detected by confocal microscopy, immunoelectron microscopy (IEM) analyses were performed on B. microti-infected murine erythrocytes using anti-BmGPI12 antibodies coupled to 10 nm gold particles. IEM analyses showed that BmGPI12 localizes to the parasite plasma membrane (PPM) as well as to the IVs and the TOVs (FIGS. 4A and 4B).

Export of BmGPI12-Containing Vesicles from B. microti-Infected Erythrocytes

The finding that B. microti produces IVs and TOVs inside infected erythrocytes and that BmGPI12 is associated with these structures led us to further investigate whether this protein is secreted into the host environment via a vesicle-mediated secretory mechanism or as a free antigen. This was achieved by subjecting plasma samples from B. microti-infected mice to ultracentrifugation at 120,000×g to separate fractions containing membrane-associated structures including vesicles and tubules (Up, ultracentrifuged pellet fraction) from those containing free proteins (Us, ultracentrifuged supernatant fraction) (FIG. 4C). As shown in FIG. 4D, BmGPI12 was found in both the Up and Us fractions, suggesting that it is released into the erythrocyte environment as a membrane associated protein, but could also be found as a free protein, most likely due to cleavage of the GPI anchor by plasma enzymes. The host protein TER-119, which associates exclusively with the erythrocyte membrane, was not found in the Up or Us fractions (FIG. 4D), demonstrating that exported proteins are the main proteins found in these fractions. Analysis of the membrane fractions by immunoelectron microscopy identified BmGPI12 associated with vesicles and tubular structures with similar sizes as those observed inside the infected erythrocytes (FIGS. 4E and 4F).

Evidence for Vesicular-Mediated Export of the Immunodominant Antigen BmIPA48 in B. microti

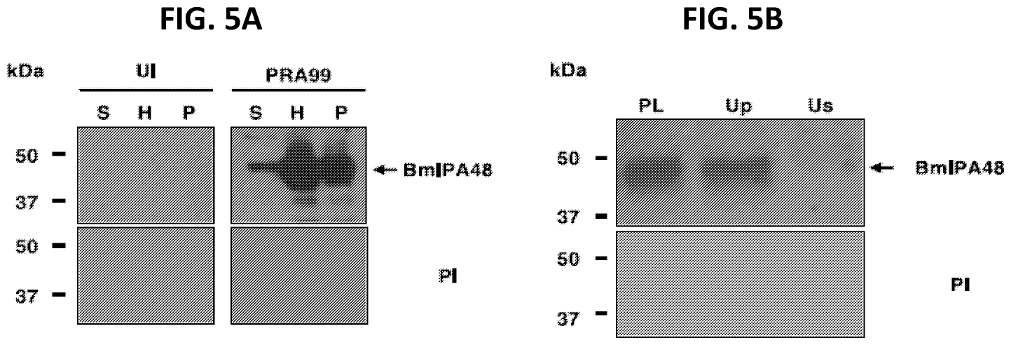

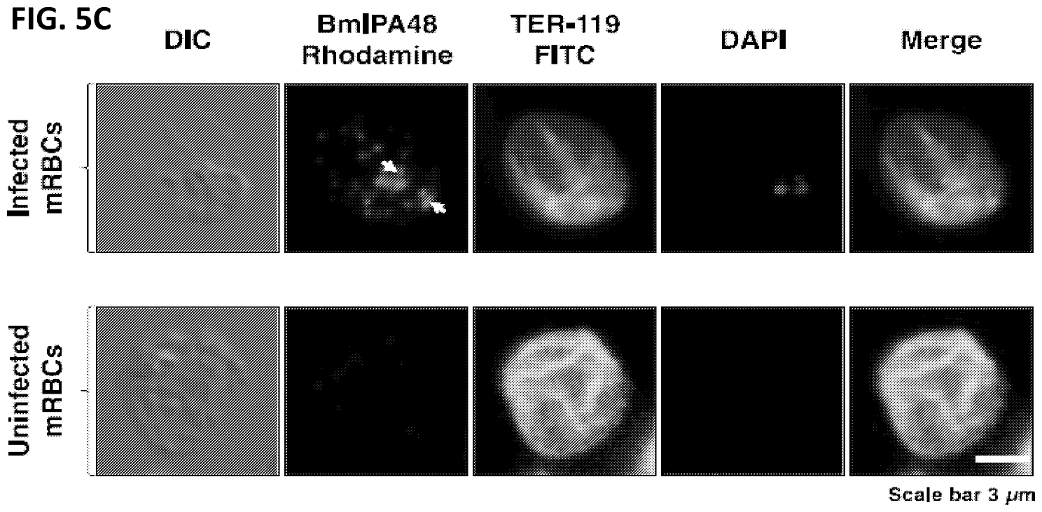

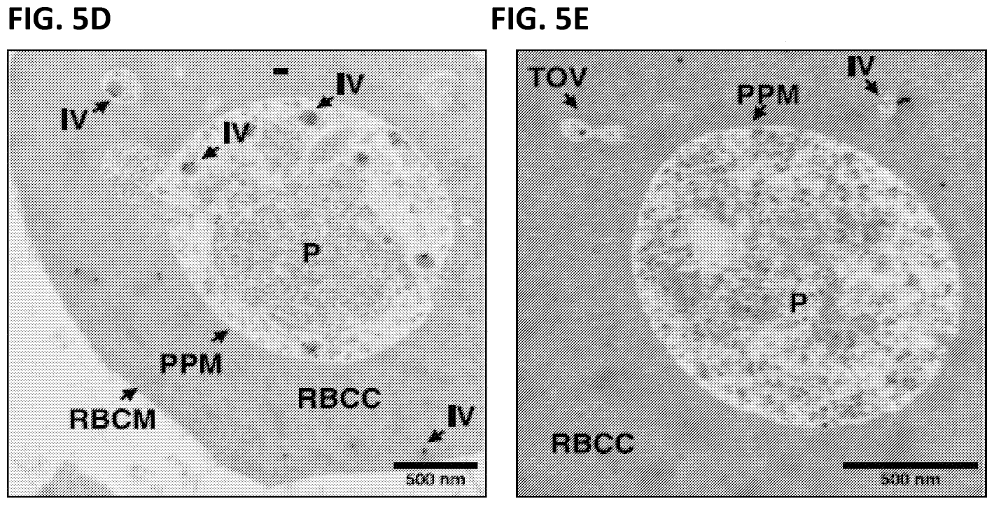

To determine whether the vesicular system used by B. microti for secretion of BmGPI12 is also used by the parasite to export other antigens, we examined the cellular distribution of another B. microti antigen BmIPA48, which was previously shown to trigger strong IgM and IgG response in infected outbred mice (Silva et al., 2016, Scientific reports, 6:35284). BmIPA48 encodes a 48-kDa antigen with an N-terminal signal peptide but no GPI-anchor motif or transmembrane domains (Silva et al., 2016, Scientific reports, 6:35284). As shown in FIG. 5, BmIPA48 is expressed in the parasite, secreted into the erythrocyte cytoplasm, and then released into the host plasma (FIG. 5A). Similar to BmGPI12, confocal microscopy shows association of the antigen with discrete foci in the infected red blood cell (FIG. 5C). However, unlike BmGPI12, analysis of the distribution of BmIPA48 following ultracentrifugation shows the presence of the protein exclusively in the vesicles-containing fraction (Up fraction) (FIG. 5B). Consistent with these findings, immunoelectron microscopy analyses of B. microti-infected erythrocytes demonstrates the presence of BmIPA48 inside vesicles found both in the parasite cytoplasm as well as secreted by the parasite into the erythrocyte cytoplasm (FIGS. 5D and 5E).

This study reports the first evidence that the human pathogen B. microti employs a novel mechanism to export parasite proteins into the host erythrocyte and subsequently into the erythrocyte environment. This system employs a network of parasite made dendrite-like membrane branches consisting of connected vesicles. We show that at least two immunodominant antigens of B. microti, BmGPI12 and BmIPA48, are exported by the parasite via this mechanism.

A recent study estimated that ˜398 proteins may be secreted by B. microti during its development within mammalian erythrocytes (Silva et al., 2016, Scientific reports, 6:35284). Some of these proteins may be exported into the erythrocyte cytoplasm or erythrocyte membrane where they may function to modulate the host cell cytoskeleton or to facilitate uptake of nutrients. Others might be further exported into the host plasma to modulate the host response or effect other changes beneficial to the parasite. Both BmGPI12 and BmIPA48 contain an N-terminal signal peptide but lack specific motifs such as the PEXEL motif found in other apicomplexan parasites and associated with secretion of proteins into the host (Cornillot et al., 2016, Transfusion, 56:2085-2099; de Koning-Ward et al., 2016, International journal for parasitology; Lanzer et al., 2006, International journal for parasitology. 36:23-36; Marti et al., 2005, J Cell Biology, 171:587-592; Pelle et al., 2015, Cell Microbiology. 17:1618-1639; Sherling and van Ooij, 2016, FEMS microbiology reviews, 40:701-721). In fact, all predicted secreted proteins of B. microti lack such a motif (Silva et al., 2016, Scientific reports, 6:35284), suggesting that this parasite might have evolved a novel mode of protein export. Interestingly, unlike P. falciparum where the PEXEL motif plays an important role in the trafficking of parasite proteins across the parasitophorous vacuole that separates the parasite from the erythrocyte cytoplasm, B. microti spends most of its intraerythrocytic development without a vacuole, thus eliminating the need for a translocon to export proteins into the host cytoplasm. Consistent with this model, analysis of the B. microti genome shows the lack of orthologs of most components of the malaria translocon (Cornillot et al., 2012, Transfusion, 56:2085-2099; Silva et al., 2016, Scientific reports, 6:35284).

The electron microscopy analyses of ultrathin sections of B. microti-infected erythrocytes showed that the interlacement of vesicles consists of individual vesicles (IVs) and tubes of vesicles (TOVs) with a diameter of ˜0.110 μm±0.0052 μm. Giemsa staining showed that the TOVs can vary in length between infected erythrocytes with some extensions several μm in diameter. Further analysis of the blood smears showed the presence of TOVs throughout the life cycle of the parasite and suggest that production of filamentous forms represent a distinct morphogenic event in the development of the parasite. Interestingly, analysis of cryosections showed that the IOV system of B. microti is of a composition similar to that of the cytoplasm of the parasite. An enlargement of a section near the parasite plasma membrane in FIG. 3B showed a tubule consisting of two vesicles directly emerging out of the parasite membrane. This distinguishes this system from the TVM system previously described in P. falciparum, which has been shown to emerge from the parasitophorous vacuolar membrane of the malaria parasite.

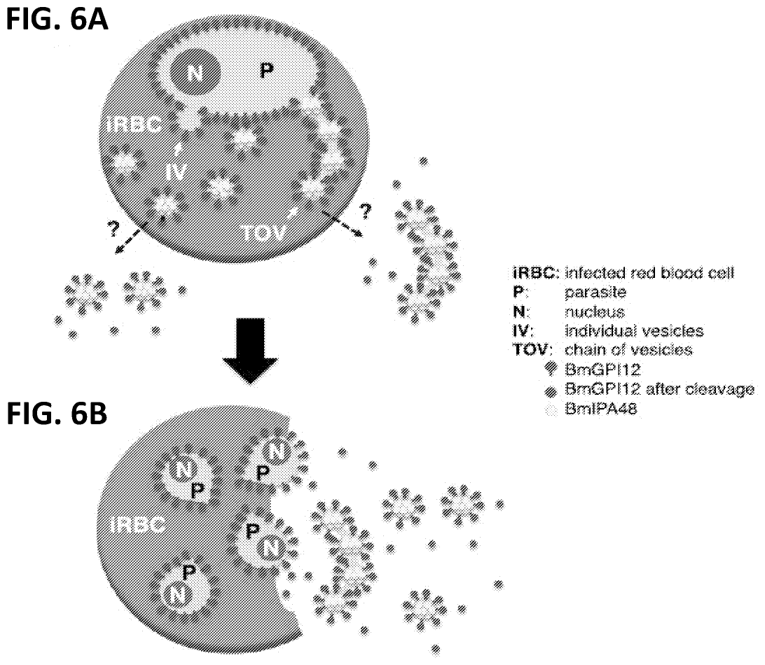

This study employed fractionation methods used to characterize the secreted vesicles produced by B. microti. Using this approach, we found that BmGP12 could readily be detected in both the soluble and membrane fractions following centrifugation of plasma samples or culture medium of short-term cultured parasites, whereas BmIPA48 was found primarily in the vesicle-rich membrane fractions. The difference in the distribution of these two proteins could be due to their respective localization in the vesicles and tubules. Immunoelectron microscopy analyses showed that BmGPI12 is primarily found on the parasite plasma membrane and on the membranes of vesicles and tubules. Interestingly, the 10 nm gold particles could be found both inside and outside these vesicles and tubules. It is, therefore, likely that following vesicular release, BmGPI12 exposed outside the vesicles is cleaved and thus found in the soluble fraction whereas the proteins still residing inside the vesicles remain associated with the membrane fraction. On the other hand, immunoelectron microscopy analysis of BmIP48 showed the presence of the protein primarily inside the vesicles, consistent with their exclusive association with the membrane fraction. Interestingly, none of the vesicles could be detected on outer layer of the erythrocyte membrane of infected cells, suggesting that the vesicles are likely released into the host environment following rupture of B. microti-infected erythrocytes as suggested in our model (FIGS. 6A-6B).

### Example 2: B. microti Secreted Antigens Identified by NANOTRAP® Proteomic Approach

TABLE 1
List of 15 exported antigens of B. microti identified using
a NANOTRAP ® proteomic approach

| B. microti Gene | Annotated protein | Protein length |
|---|---|---|
| BmR1_04g08775 | Putative HtpG | 712 |
| BmR1_03g03490 | Putative EF-1 alpha subunit | 447 |
| BmR1_04g05965 | Putative Enolase | 438 |
| BMR1_03g01010 | Putative Ubiquitin family | 77 |
| BMR1_03g01340 | Putative Ubiquitin C | 282 |
| BMR1_03g02390 | Putative Actin | 376 |
| BMR1_02g03540 | Putative RPL18 | 187 |
| BmR1_04g09800 | Multifunctional tryptophan | 425 |
| | biosynthesis protein | |
| BMR1_01g02545 | Hsp70 | 644 |
| BMR1_02g03395 | Unknown function | 395 |
| BMR1_03g04775 | S-adenosylmethionine | 404 |
| | synthetase | |
| BMR1_03g03380 | Glutamate dehydrogenase | 467 |
| BmR1_04g08050 | peptide chain release | 433 |
| | factor eRF subunit 1 | |
| BMR1_02g01430 | Unknown function | 1094 |
| BmR1_04g08520 | Unknown function | 1024 |

### Example 3: B. duncani Secreted Antigens Identified by NANOTRAP® Proteomic Approach

TABLE 2
List of 46 exported proteins of B. duncani identified
by a NANOTRAP ®-based proteomic approach.

| B. dunani Gene | Protein Length | Annotation |
|---|---|---|
| BdWA1_II1364 | 229 | Hypothetical protein |
| BdWA1_II1520 | 644 | Hsp70 (BdHsp70-1) |
| BdWA1_III2507 | 1058 | Dynamin family, putative |
| BdWA1_I0004 | 183 | Hypothetical protein |
| BdWA1_I0706 | 376 | Peptidylprolyl isomerase, putative |
| BdWA1_II1893 | 160 | Hypothetical protein |
| BdWA1_III3045 | 132 | BdGPI6-Hypothetical protein |
| BdWA1_I0760 | 188 | RPL22p/L17e, putative |
| BdWA1_III2950 | 490 | Protein disulfide isomerase |
| | | related protein |
| BdWA1_II2303 | 442 | Enolase |
| BdWA1_I0910 | 177 | Hypothetical protein |
| BdWA1_I0555 | 131 | Polyubiquitin, putative |
| BdWA1_II1366 | 229 | Hypothetical protein |
| BdWA1_I0089 | 797 | Metallo-beta-lactamase |
| | | superfamily, putative |
| BdWA1_III2562 | 392 | Alpha/beta hydrolase family, |
| | | putative |
| BdWA1_I0812 | 713 | Hsp90 (BdHsp90-1) |
| BdWA1_III2693 | 126 | Hypothetical protein |
| BdWA1_II1642 | 255 | 14-3-3 protein, putative |
| BdWA1_II2273 | 742 | RAP domain/Core histone |
| | | H2A/H2B/H3/H4, putative |
| BdWA1_III2537 | 400 | Rhoptry-associated protein 1 |
| | | (RAP-1), putative |
| BdWA1_II1831 | 242 | BdGPI8-Hypothetical protein |
| BdWA1_II2038 | 917 | RPL1p/L10e family, putative |
| BdWA1_I0231 | 618 | BdGPI17-Hypothetical protein |
| BdWA1_II1370 | 266 | Hypothetical protein |
| BdWA1_I0253 | 498 | Hypothetical protein |
| BdWA1_III2944 | 296 | phosphatidylinositol-4-phosphate |
| | | 5-kinase, putative |
| BdWA1_I0708 | 376 | Actin, putative |
| BdWA1_II2269 | 292 | EF-hand domain/EF-hand |
| | | domain pair/EF hand, putative |
| BdWA1_II2320 | 476 | Sodium: dicarboxylate |
| | | symporter family, putative |
| BdWA1_III3035 | 207 | Hypothetical protein |
| BdWA1_I0924 | 299 | Ethanolamine-phosphate |
| | | cytidylyltransferase, putative |
| BdWA1_III2663 | 516 | MAC/Perforin domain |
| | | containing protein, putative |
| BdWA1_II2261 | 1416 | RNA polymerase Rpb1, |
| | | domain containing protein |
| BdWA1_III3073 | 333 | PP-loop family, putative |
| BdWA1_II2013 | 278 | 5′-3′ exonuclease, putative |
| BdWA1_I0563 | 825 | Hypothetical protein |
| BdWA1_II2256 | 374 | Hypothetical protein |
| BdWA1_II2291 | 535 | Hsp70 (BdHsp70-2) |
| BdWA1_II1496 | 400 | elF4A-3 |
| BdWA1_II2200 | 108 | L7Ae |
| BdWA1_I0250 | 418 | 26S protease regulatory subunit 6A |
| BdWA1_II1994 | 524 | GMP synthase |
| BdWA1_III2590 | 126 | GTP-binding nuclear protein Ran |
| BdWA1_I0810 | 612 | Hsp70 (BdHsp70-3) |
| BdWA1_II1634 | 117 | Core histone H2A/H2B/H3/H4, |
| | | putative |
| BdWA1_II1773 | 396 | 26S protease regulatory subunit 4 |

### Example 4. Babesia duncani Secreted Antigens: SEQ ID NOs: 1-46

TABLE 3
B. duncani secreted antigens identified in the pellet fraction after
ultracentrifugation of the culture supernatant: SEQ ID NOs: 1-23

| | | SEQ ID |
|---|---|---|
| Antigen ID | Annotated Sequence | NO: |
| BdWA1_II1364, | MKFLFGFFVILFLRLSKQEELVSLQLGDFHFDFEN | 1 |
| hypothetical protein | GKYSTHEPFETCFIDVYNYRYDKTGPFLFNIFIKRT | |
| | LENEYQSLFFKRENGKLVNFAPSHLSSPQTDNTGT | |
| | YYSTKEPVVLESKNLSDIRNGIKKIGGNKLSSSGKI | |
| | NWDTISTTLLLKTACGTYSTYSSGYEALLPVKDG | |
| | NDTFCCCFSKAVLFTGYRFQMKKYEEVKPASNSQ | |
| | STCKK | |
| BdWA1_II1520, | MAATAIGIDLGTTYSCVAVYKDNNVEIIPNDQGN | 2 |
| heat shock protein | RTTPSYVAFTDTERLVGDAAKNQEARNPENTVFD | |
| 70 precursor | VKRLIGRRFDDPTVQSDMKHWPFKVNAGAGCKP | |
| | TIEVTFEGQKKTFHPEEISSMVLIKMKEIAEAYLGR | |
| | PVTDAVITVPAYFNDSQRQATKDAGTIAGLNVMR | |
| | IINEPTAAAIAYGLDKKGSTEKNILIFDLGGGTFDV | |
| | SILTIEDGIFEVKATTGDTHLGGEDFDNVLVEHCV | |
| | RDFMRMNGGKNLATNKRALRRLRTHCERAKRV | |
| | LSSSTQATIELDSLFEGIDYNTTISRARFEEMCNEK | |
| | FRSTLIPVEKALRDADMDKRKINEVVLVGGSTRIP | |
| | KIQQLIKDFFNGKEPSRSINPDEAVAYGAAVQAA | |
| | VLSGNQSEKIQELLLLDVAPLSLGLETAGGVMTV | |
| | LIKRNTTIPTKKTQIFTTNEDRQEGVLIQVFEGERA | |
| | MTKDNNLLGKFHLSGIAPAPRGVPQIEVTFDIDAN | |
| | G1LNVTAMDKSTGKSEQVTITNDKGRLSQTDIDR | |
| | MVAEAEKFKEEDERRKCCIESKHKLENYLYSMRS | |
| | TLNEDAVKQKLSTEELQNGLNTVEEAIKWVENN | |
| | QLANQDEFEDKLKEVEKACAPLTAKMYQAAGG | |
| | AGAGGMPGNFGGAAAPPSGGPTVEEVD | |
| BdWA1_III2507, | MATIRKQRLEDLTDIHKEHLSTAHQLLDVIKSASD | 3 |
| Dynamin family | PKLIYLHCYQLMKLGGLDAEVPRLVVFGQQSMG | |
| | KTTLLDFIMGGPIGYSSTDTGTRQPVVIIMRPESAI | |
| | DPRELELASGSVGSPSSTITSKKIWCKFNGKIMDIH | |
| | NVQQNMRLHMQSLGERICSEELEVEVYVPDAITA | |
| | IFVDLPGIKDDSKSGAEFTRRVVRNYVSNNPNDL | |
| | YLLVKKSSDDPANWPWSLREFITAAAPNGLGLSP | |
| | QQTMVVGTRAREFLINEKTDIRTQDQLYERVHKR | |
| | AVLDSKGQALPLHLLELFSLSIQAKDKGDFLTNKE | |
| | EMKRQIANGQVEVENMIRHGFEESNSINKEGRSV | |
| | SEELLDTFSIRQFLRSLNSKFSQLLNGHLTNLERRL | |
| | IRKKIDLERIVASLETKLQCFSPTTVRESIKQFIRQF | |
| | MEIVHNMMMGNYTIMKLPIPPEQFLGIYGGSLRD | |
| | NLEDGHELAQNLFPQPDMYESNFYTKITARTEEL | |
| | YNKKLTMIDTVKPGRYVRYFTSKISVHMFGLIEPP | |
| | RRVPPAVSGADLGPFDMMGQDYGADELINVEFIQ | |
| | LNNNEENSLHKDIDRSKLSLLTPLASVSAEHLQVP | |
| | LYAWHKTKEPKTGWVVVRPIVIDRLPPEILVQKS | |
| | NYREVDVANKQISFRYLDVEFETKALGSGEEDQL | |
| | ENSVETEISETPPVVEGVQNRCSNRYLYVTACSEI | |
| | FLEEHRTAPYYASALEMVSGEHAEANLLNQLAVT | |
| | NICHWLKFQIKHMEPEHVYSAEVLYQMLRSIDHV | |
| | VDRADWEPLVADLVQSNVRGTLLHASRLAACAS | |
| | AAALRRVLRASLAEAFRCIKLTDCDQTLYCLPDS | |
| | LHFQEQIDHLSEEYCRQKAIDCANAMMNCIIEQT | |
| | YSIQFDVAVDIFDGCRQFEKYFMGRAGHRSFMGD | |
| | ALSSVKEDLALRKRRLAMTDIFEKSDAKTSIELIY | |
| | EEVKVQFWATKLLLSTPLTTKLYTYFIKQVVDKA | |
| | LPTTHSDPTASIKVEFEQFLIDNILYEQIDGTRTAK | |
| | SNNRLSADYDLNNKYEQLVQQHNRNKRLLEYISC | |
| | ALEGISRFKHHATADTDFLAHLD | |
| BdWA1_I0004, | MISSPPSGFSLHGEDAKGTPRDVERQDSDRLDPG | 4 |
| hypothetical protein | NFPPDKYWSPSIGLSTIEANRRVVWLLQEAISRYK | |
| | IHLGYWNTTKSCLTIDILIGVLVLVLFILGAEPNLG | |
| | VWHVVRPVLLLPWLVLNCYSKITVMRIKSSRACE | |
| | RVRDVLESFSRELKTGGHTEVEQRRFSFDPPNSLL | |
| | IPVYRDREWKRLPANVLLAGDVFKLQIGDYFPCN | |
| | CRIILSCDREGKVQLDANLFNAGSVFKSSHLPSINT | |
| | NDGGEWTDASFVAVTDSFVQSLETFLSADQGPQS | |
| | RFMFFNERDWENCQKGPNTPPPTTCVWNFSDYT | |
| | HFSKYGVDWIQLGIAFFISSIVTVLQIIWSGFQGWR | |
| | RHVNVFATCIICLCHPAFDPFLNLADIWGNVKLQS | |
| | LFQWHNEKRFTVDILPQQSSSSSSTFDSGSSEDSV | |
| | DSEMAASRIPLLHQLRELNRVFKRGLDSEGSLLRT | |
| | LCSVTLLCFVDDMGLLTEGCATPQELAVVDPSGG | |
| | IGTQRRQQKESTAMFQSGNIDAIETINSTATSHKD | |
| | SIGQTEQSDKGTIKAGPREGKQVVRKDPQGGERL | |
| | VILDVFEDSSQYYKQYVKFNNDAERNCIPQVLSL | |
| | SFAMSATQFPRVQPSLLQLKAAPDLIHTYMGMLA | |
| | NGTLHDFTHCLCAFAGSVGLKRSYIRRFRLLRFIV | |
| | VLDETLGTNGKMLIYFLRDPRKQIVQMLVKAKPE | |
| | TVFDRSINYHDRARGAILPVSRLTKRKLRDLNMQ | |
| | WVSSGLTPFAFIYKPIHLDEFNLIMAHLPGVAVFK | |
| | VGHFLKLDRHQSVKYQESTTCQQDPQADYFGEG | |
| | GKSDAKARRWYARLTDYDAISMRYGSMHTDLR | |
| | QRILTNSIGGLVNSCLKNSILLGMCATKYQYPKEV | |
| | PSRIQSFHEAGIRFVYFSKHDEKQTRIVGGLLGLET | |
| | SWNSMISLVKSGRYSHVNQDGRVVLPSGIDNIRR | |
| | HIRDVDDIPLQVSLFCDCTHTSTVEMMRILRENGE | |
| | RIMCVGNGLRPSNFFVFCEAHSSVSVALGYHPTC | |
| | RFCRGKRWSGIARAHAFEEATPEMKLSAFLTSLP | |
| | CDLQTSKMYKVADPYFVMEMLHEVFKEARHMA | |
| | TNIQDAAAFFKLASHSVAWMLMFQASLGFRRIL | |
| | MPADLALLIFVYIPLMGSCLLSNVVAEGTMQQMP | |
| | SRCTKDDAKVTIAAMCKTYPRLLLVATSLLVFYS | |
| | FVLGQIQALLKIELERLYNFTLDDTQCSRFWRVAS | |
| | YSCLQEHEQSIALLRTQVQSVIFRENTAAHLAEQT | |
| | ASFAMAFLYSVSSASWIVRTGRIGAIASELLHSRW | |
| | IISSAAILTIQGTILVARILAQPVPLASLNAIMPWGL | |
| | VCGMLLGLSLAILAVDHLHKFYAARQHEMDQKN | |
| | VESKVTSDLDIMDVTCICEPILSPSTQKISKRTSPPP | |
| | IKQNVRGILQCKKRSYTSKSNRRRHPAIFNPDTTS | |
| | AKDMGVLRTRQHYTSILLHFLQRGINGEQLQLVS | |
| | LDIDQESFRLPLKKHSFGEEAAPAFLDVYVEEDIQ | |
| | KIPVMFNLVIETKGDYTYRNYFFKRDGDKFINFD | |
| | VPKLSDYYNVRTEGIATYYLTYKPIRVPREVLKRL | |
| | RREFPDNNQLKVSNDPEDGPRSNEITLKLKLNSGS | |
| | LVSYMGTYVPKTSSRGCRSKAPIFNKFRYKLTTD | |
| | GQAIPVDEVEEEGEEEEEENENEKTEETDDQDRK | |
| | VVTKEDDEEEEDEEVEEVEEELVEIKDEEEDKCYI | |
| | EAWEKHSKNYSKVEVATSNKKHISDTGSLKPIDE | |
| | LSGLFNALERKIATETALESDTTYTPKDNERWIID | |
| | KCNSEESILDLHIKEDILTFDVPITQLPIQEKEIVDP | |
| | CNSQRKSAFFNKIGSAGEHYLRAFGSNILSVIDIM | |
| | KSDLNVSIKKVNSDEINEDLPTKEKLLAMEPIMFN | |
| | SMDGEDNNNKNTDEEVNGTKTLMHSINSQSIERC | |
| | EMNSAYNQGSFISIHDGTSDSFMDSDSFSISQKITQ | |
| | DELLSKHDDKTLLNTDNALNDDSGLKTTEISHTV | |
| | NDTVIPNLKLEKLLPQSKEQSAESTSSGEVGLIKK | |
| | NKDVQSTKGKVGDLGNYVQINDDKEHPREIVTV | |
| | EKFADETGIGTSTKKNEPATTSPLNEFDKIPNNDA | |
| | AVNDSEKKTPADESRGNTNAKKTSDGNFKTISAN | |
| | LKPLKPFRPDSLVTAMHISNDNKFLATGNENGEL | |
| | YVWKFESAKMMSSPEISDNRINALPKMLNWEML | |
| | SSTPTAGVQAHEFFIYSIHISKIQNRKHSGSVLVVT | |
| | SGGNNYVRIWALTKSDKENVLILKGDRQFDDDIL | |
| | TAFQLPISQHIAICGIDQVIEIWKFPPITTSVNDNPK | |
| | NISSWNKTKERIDVELTIQSVSYSPSGMYIAIGSID | |
| | GLLSLYSAETMKLISMAICRNEKGWYSNSASITGI | |
| | VWNTKETLVCATTADSRIRLFSTNIENDNALIYAE | |
| | KLKGHKYCGENVAARFTGLNDEYVICISKGGYIVI | |
| | WKHSCTEERIFYDGNPIIKNTNYCKFKIIPKPYIGK | |
| | LLGVFNPGTWGHLWPTYQEQSVYEPLNDKNSVG | |
| | CLDRFLTKNTPGVKFSKANPKNAILLVASVDGSQ | |
| | LLCTIIDASLLVFKKS | |
| BdWA1_I0706, | MTESVDLTGDGGVLKTIIKPSKFFEQPEQGHEVEV | 5 |
| peptidylprolyl | HYTGKLDTGFVFDSSHKRQTTFKFVLGDGNVIKG | |
| isomerase | WEVGVAAMNIGETALLVIKPEYGYGAAGSGSTIP | |
| | PNATLHFEIELINSRIKPKQKWEMSMDEKIQAAAD | |
| | AKVEGNAKFSQERILAAITYYEDGISYLSTRDEWP | |
| | EDAIKASNVTKLQCHLNLANCYIKTEDFANAETH | |
| | ATEALNIDPVNIKGLYRRGLARVKQGDFKEAIDD | |
| | LSKLIKIEPTNAAGVSQLKLAKEKYTIFKQRERNV | |
| | FGSVFKKMNLYDEKSGIRNLDTLPRVFLDISITGH | |
| | VDVNRLVIALFEDTVPKTVENFKSLCDENAKLTF | |
| | KGNKFHRLIKGFMIQGGDITNGDGTGGACIYGDQ | |
| | FDDEGFKDSHSERGLLSMANCGPNTNNSQFFITFV | |
| | PTKHLDGKHVVFGKIVEGMEFLDILENLSTGEND | |
| | RPEADVTIEHCGTL | |
| BdWA1_II1893, | MPSQGTVKAQTGTTEVDNADSSEEESQKSQPESK | 6 |
| hypothetical protein | AVNGISGTESRNENSQSVDTNSSGDTNPSQSAGGS | |
| | APTTGDSKNQQGKNVNAESSSNPNSEKSVATQDS | |
| | STDQTGKTDSSTTHTTTGDSVEQKGDDNTETTDT | |
| | AQNTATEEATTGSGTEGSADQTE | |
| BdWA1_III3045, | MARFFSYKKLIAFAIVALASLKEVSFLGGCPYALA | 7 |
| hypothetical protein | VATTTTTGTNGAATGTNGAATGTNGAGANDTSK | |
| | NTSDPNTPATPPSSPESNKDNAAGGSDGQKPTGQ | |
| | DPQKPNAGNGFAATSVIGAATIGLLTLAFN | |
| BdWA1_I0760, | MTKYSQEPSNLAKSAKAYGAHLRVHFKNTYETG | 8 |
| Ribosomal protein | RAIQGKMILEAKRYLNDVIEHKRCVPFRKFNGGV | |
| L22p/L17e | GRCAQAKAFKHTQGRWPEKSCRILLDLLTNLESN | |
| | AEAKGLDVENMVIENVLVNRAPLGRRRSYRAHG | |
| | RIIPFLSHPCHVALIAVEKDENVPRFTPEAAKTIKL | |
| | NKRQIARMRLCNGKGVAK | |
| BdWA1_III2950, | MISHYCLLLLAAATVFGGEATVTIEDAKSATLEAT | 9 |
| protein disulfide | TGVPEVAEGDVSIPRGVVTLTADDLHKSIEKHEAI | |
| isomerase related | MIKFYATWCGHCKILAPEYIKAAKILEEENVNVV | |
| protein | LAEIDAVAHSDAVAEFEIKGYPTIKFFKRGIPIDYN | |
| | SDRKAETIASWCKEMLNPALMETTNLEAEIASRK | |
| | SKIALVAHGCNDKDELCVLFEKLAEVHRMDAHF | |
| | FSVADSSSVWFEVRHVGDGTLKFNGLSPEELALF | |
| | VKDETLPLLDEINPANYARYTSSGKSISWLCANTQ | |
| | DYTKYRSSIVEVAKEMRSHTVFVWLDTEKFSAV | |
| | NEAFAISKLPAIAHQTMKGRFILSPDAYDFTSKSA | |
| | MLQFYTDVEQGKIPLSFRSEAEPQDATEGPVMLV | |
| | VGKTLQQLFTQTDKAVLLMIHAPYCEHCRNFMP | |
| | VFEDFAKTIDAQAPLIVAKLDGDANESPLDYVSW | |
| | EAFPTVLLFKAGDKQPIPFKGTRTIEELTSFVQEHV | |
| | TLAPVKTEL | |
| BdWA1_II2303, | MVEELDGTKNEYGFCKSKLGANAILVVSMAAAR | 10 |
| Enolase, N-terminal | AAAAHLNIPLYVHLANLAGKPTNKFILPVPCLNVI | |
| domain/Enolase, C- | NGGSHAGNMLAMQEFMILPIGAGSFREAIQMGSE | |
| terminal TIM barrel | VYHTLKKVISSKYGQDATNIGDEGGFAPNIKNAE | |
| domain containing | EALDLLLEAFRIAGVEGLFKIAMDVAASEFYDKN | |
| protein | TGMYNLGFKGKEPQNKTGEEMISYYKLLCAKYPI | |
| | CSIEDPFDQDDFDSYTKLTAAIGEKVQIVGDDLLV | |
| | TNPKRIEMALGKKACNALLLKVNQIGSVTESIDA | |
| | CKMAHANKWGVMVSHRSGETEDTFIADLVVAL | |
| | GTGQIKTGAPCRSERNAKYNQLLRIEEELGNKCE | |
| | YAGHNFRTCGN | |
| BdWA1_I0910, | MEEWYTYALRHVDLDLDNFFVEFGETILEDYTRI | 11 |
| hypothetical protein | YPRSNVFVDNIRKGATLITLSTEHKGLLFVELRYP | |
| | RPGKDSVMDIIYKNWEDEEVMFSLVWVFGDWVP | |
| | QNFLYSGILDSGPEYVPVRRHDSR | |
| BdWA1_I0555, | MQIFVKTLTGKTITLEVEPSDTIENVKAKIQDKEGI | 12 |
| polyubiquitin | PPDQQRLIFAGKQLEDGRTLSDYNIQKESTLHLVL | |
| | RLRGGVIEPSLVILAQKYNCEKMVCRRCYARLPL | |
| | RATNCRKKRCGRCSQLRPKKKIKGGN | |
| BdWA1_II1366, | MGNPGLIFIALFKYSFNCYYTFLPFKFDNNHFDVL | 13 |
| hypothetical protein | PHENFLPRATVPRFVDIYVSADDPRIPILVNFVTGG | |
| | MTTSGPDTRPTNRNYFFKRDGNKLVNYKFTNKP | |
| | NDTVENNDIVTRISRERVTYYVGTSNPEDIIVATK | |
| | DPQNDLNQRENSMYASLATLDPKATIPRIGLKTK | |
| | SKYNKVALYSDEHKEKIEISNADIDGVMHEKLILF | |
| | RYRLVKEYRV | |
| BdWA1_I0089, | MRNVKKRTVPFLWISCFVLYGIYNVEPIALYNKN | 14 |
| Metallo-beta- | WVHKRVGFIANLKQPPCGITFQGLTHEKAKRGFK | |
| lactamase | RYAEQSSQVKEASVLFDTLSNTEVLDELDSNDAI | |
| superfamily/Beta- | QDTPDPLESETIIEVEVEDKGKKTFSDIHNKLKGSI | |
| lactamase | NKLPSTFAVMKDLVAFLDTMEQQGNEKAKSIAY | |
| superfamily domain | TSIKKKLITELNKNVEELKSDLPKTKKRLYISPFLK | |
| containing protein | NTVDPFFEDQYVNPVDFKNYFSLYDNYKQEYRKI | |
| | AYLCMKTYIARLILGLKRKLKHGIVSTFLWPFAK | |
| | WAKDNNVRYKEPKPSQLENFERLLKYYGHEFEN | |
| | VYFEQNIAMVRPPQPKARPPNKWSLIFLGTGSRQP | |
| | TDTRMTSTMAFTEHDGGRIWLFDCGEGTCACMQ | |
| | KLNLNPKAVDRIFITHLHGDHCFGLFSFISNSARA | |
| | LPITVYGPIGISKMLIDIMNFTTTSVLPKFVVHELV | |
| | LHPDNEKHKTGWHVNYPFGGYIYPQEAGHYLV | |
| | YENDTCKVMAAPLKHILPTVGYVIKEKSKNENDS | |
| | TKTQRKIVICQDSCDSSKMVPISMNPNVLIHEATT | |
| | STTSTIGSSLIMQLVYNFSKGKIDESLLSKINDIISQ | |
| | EELRRSCFSVTFTTMAMKLRRKCYLIERSYSNLTK | |
| | TLEQNELKATETESNNADMMTDIYNIVQLVHAAS | |
| | KIKSCIAEAKKIESQLKELYDAPKHLGPRSTWLKS | |
| | VYNHVRDVIENNGNTETSSSPTIQAMESLKNLLTK | |
| | FWTTNKSMGLLFSLEINLPEAAPKDWFSLYSKSV | |
| | RYSGHSTPWDAGKFAAKINAASLYLTHLSSVSLH | |
| | VHFY | |
| BdWA1_III2562, | MLLSCWFNLIACFICICSCYIRLNQTNCTNLRLINT | 15 |
| Alpha/beta | NNSNIKMKATGKLSMSHFKNKQGLLIRTYAAEV | |
| hydrolase family | DKPKGSAILVHGNKSHFRADFTNYNVDFYKDKY | |
| | GLESVDPNIVIREMHAIYPNVDHKIDFNNDYEFHY | |
| | SKLDGKNALDITPRFTLNGSIVEYLNGLGYSAYGL | |
| | DLQSQGMSQGHNGHRNYFKKFDDHVVDVIQFIDI | |
| | IRRNKFHNVNEEWDPNVLGKNYSLNKCFLMGLS | |
| | MGGNVVLRAAQIFKTLSDFKSNIVDGIVCFAPML | |
| | DIDMHFSGAFNQLALAIAKMIVTFCHHSTFMINEK | |
| | YEIDTLNSFLRVNDPYYITKTQTHKAIVSLLEATR | |
| | TLEKNHSKYPVDMPTLVFHCKDDNVCDFKVFTC | |
| | NEMI | |
| BdWA1_I0812, | MADGMNQETYAFNADISQLLSLIINAFYSNKEIFL | 16 |
| heat shock protein | RELISNASDALEKIRYEAIKDPSITEDQPEYFIKLYA | |
| 90 | DKNNNTLTIEDSGIGMTKADLINNLGTIAKSGTKA | |
| | FMEAIQAGTDMSMIGQFGVGFYSAYLVADKVTV | |
| | VSKNNNDEQYIWESNASGHFTITKDESGEQLKRG | |
| | TRIILSLKDDQTEYLEERRLKELVKKHSEFIGFPIQ | |
| | LSVEKTTETEVTDDEAEESADAEGEKDKIQDVTD | |
| | KDETEAKEGEEGDKDKKKKKRKVQNVTREWEM | |
| | LNKQKPIWMRSPNEVTNEEYASFYKNLSNDWED | |
| | HLAVKHFSVEGQLEFRALLFVPKRAPFDMFESRK | |
| | KKNNIKLYVRRVFIMDDCEELIPEWLGFIKGVVDS | |
| | EDLPLNISREVLQQNKILKVIRKNLVKKCLELFNE | |
| | LTEKKDDFKKFYEQFSKNLKLGIHEDNANRTKISE | |
| | LLRFETTKSGDEAISLRDYVDRMKPEQKYIYYITG | |
| | ESKQSVANSPFLETLRQRGMEVIYMTDPIDEYAV | |
| | QQIKEFEGKKLKCCTKENLELDDDEEANKNFEKL | |
| | KEEMEPLCKLIKEILHDKVEKVTCGRRFTESPCAL | |
| | VTSEFGWSANMERIMKAQALRDPSITSYMVSKKT | |
| | MELNPRHAIVRELRQRAESDKTDKTLKDLVWLL | |
| | YDTALLTSGFNLDAPAEFGNRIYKMIKLGLSLDD | |
| | DVAEASLDEVPALDEVPVDSRMEEVD | |
| BdWA1_III2693, | MRTLSRLPPPGDPGHVASFQEIATFNIDEFLANIDK | 17 |
| hypothetical protein | THDGVSQLQLPPNLLENLKQGAGSLLQHLAEQST | |
| | GPFATHNQPSLIGASGMAVAAPQMQLPQVAMAP | |
| | QVMAAPQPGMPPAVAMPQMTSQFAGAPGQALP | |
| | VAAQAVPNMQAPAGPQLASVPISNVIRGNNIAFP | |
| | GAVPNEELAMELVMKITWALLMHC | |
| BdWA1_II1642, | MSEENSQRAQLTYSAKLAEQAERYDEMADAMK | 18 |
| 14-3-3 protein | LLVETCITDKDELTVEERNLLSVAYKNAVGSRRA | |
| | SWRIVSSVEQKEASKSNSVHKTLAGEYRAKIEKE | |
| | LNKICLCIIGLLDEKLIPATVDSESHVFYYKMKGD | |
| | YYRYISEFSCDESKANASASARDSYQKATEIAESE | |
| | LKSTHPIRLGLALNYSVFFYEILNRPQQACEMAKR | |
| | AFDDAITEFDSVSEDSYKDSTLIMQLLRDNLTLWC | |
| | SDVTSDAPDKQKQED | |
| BdWA1_II2273, | MFGLLRPCLIRLSGAVATRQTFGNLADCLKIAESS | 19 |
| RAP domain/Core | QSIATTDLYEAFKFISSSGQIRRQAIHDDRFVTLLD | |
| histone | QLDARISTLNCSYMGNFGIRLGLIIQSLGNLDRED | |
| H2A/H2B/H3/H4 | PIVEKSVKVIERLCTEMMEKSGNIKEISQLAFAAA | |
| | SAGLQHKFLDYAKQNLTLNIENADPDVLNLALLA | |
| | SYKTKVHDKVFLALICEKLSELTDRFTANDVVST | |
| | LRSLEKTSLMKGFLLRRLSMLIHDNLEQFTNEQLA | |
| | QCCYRLSILKFQTPVQYSTILSLLEPKFQQLSIHLQI | |
| | EVLASGCMCQCTDANERLVKLAKSITLTDKVDLA | |
| | GLVNYIYSCVYLKLYKGDHLTGALEEALARSPFLI | |
| | RKYALLFKEAYDTLSLECPNLTLELPEAWKMALE | |
| | NYESAEHDRCIQTSIIAETGNILKTSAGDFETFSKV | |
| | GPFTVAFADVARKLVILAETPNTLGGLALAQRSIK | |
| | AMDYKVAIIKYWEWRRLKTEKSELSYAFKRFDK | |
| | SRLGVLSHVQFVRLLGAIGIHLTRQELKFLQFEEDI | |
| | RGGFTLEDLEALGRDFYNDEVIATKVLESLQEHF | |
| | GPCNTLDKQELASVLMKLGASLGVAREELDTFLN | |
| | FYSAHSNSISVEAFIRGAALGVLELCADHTVTILQ | |
| | WETSRLQTKTSKMSGRGKGGKGLGKGGAKRHR | |
| | KVLRDNIQGITKPAIRRLARRGGVKRISGLIYEEVR | |
| | GVLKVFLENVIRDAVTYTEHARRKTVTAMDIVYS | |
| | LKRQGRTLYGFGG | |
| BdWA1_III2537, | MVLSPILQFSFLALPSMLLNNVFALRMSAPTPIESP | 20 |
| Rhoptry-associated | VTKYGDSLNMLFLDLGNAFHENFSMFQVATSMS | |
| protein 1 (RAP-1) | NYAETNNDIVSRICERFESQKACFVLASKYINNCA | |
| | KAKKCMQIEKFHLFQSPDMSIKLMNRAQLAAAIH | |
| | VFRNSGVYEKNYLKRRFNKIFKRQPFGYSSYRTL | |
| | LIPLLYSNASFNEYTTFSEIFITYYLNVATFMYATLI | |
| | YRDTRLARFVNAFDKLNVLLIPLKKHLRNMVTGI | |
| | ANASPVAFCEEDFEPILRIFGHYLSGFDKSLTPLAN | |
| | RFAKLIRDVLKNELHKSESCILNRAGDFLKNAGR | |
| | RAGKAVRDAGATIKERGMATYKGVKGGLAGAS | |
| | DKIRNRFRSRNGNGDDASSYGLLDEDATGEATDD | |
| | VKPEDDSTGDNEPKEDEITRL | |
| BdWA1_II1831, | MNLKWLLGLALIGSKYALGGDPNDSEVDSGKER | 21 |
| hypothetical protein | GPGKRMTFDELLDELKTAEASVLGIKAEINGGLN | |
| | RLRYRIGNLDAITKSDYDEISDAIRDIITKRTEFAK | |
| | AVNKRVQLEAIANKFSERTSMGNLEDIQFSTFWV | |
| | KLEAITRVPDFQLKEDFVKMKDEIIDVKEKFIEKL | |
| | KKAREATAEVIPETIVEDQEMKSDLHEEIKSHGDD | |
| | DIFNDKSDKKQNSGFAATSSSLILLAMATIGYSLF | |
| BdWA1_II2038, | MAKSALYILDSNVKSKKVDKESLKLIKKQSGKPK | 22 |
| Ribosomal protein | KILRPVEHKAHNPSCLENAKVELDLDLLKKVIETL | |
| L1p/L10e family | KKRAEVVRESNTRDLLEDPSRNYVFIQIALTKVVT | |
| | EVHVKPLQIKLKHPIYTDKEVCIFVKDPQKHWKEI | |
| | IKRENVPQIKKVIGVTKLKKKYKQFEDRRKLCRSF | |
| | DLFLCDKAVCCSLPSLLGKVFIQRKKMPVPISMSK | |
| | GGLGNSMREAIQSTYYKLSTGNTCSVKVGICSMN | |
| | TEQLIDNIKQVFQTIKKFHTEDPIFRNVISSIFLNWE | |
| | GTESLMLYSRALADDDIQIPQSHVTSPSKPTAAKP | |
| | TCKWFGTSQSDLVARIVATAKGGTTSLSVWRQFS | |
| | KDVIETIDTLNIPDVYRILKCFSILRYRHDPLLNVIS | |
| | YRIVESLDKIACKNLAEILKAYSKLECRNDFLLKT | |
| | ALPTVARHLEFFTPSDLSSVFYSYCNLGFHDLNFI | |
| | RQVEWRIFNTLGKLQSCDFALLFCGLTRLERINTR | |
| | FVISLACQFCKSLDAIDEKHFSLCVNALGRLEFAE | |
| | HPHLYGILVQHIYNEIKLHHLTSVSLALLVNGFSR | |
| | AKPKDLKVFQMLSKQIESRLSEFDIHSLCLITAGY | |
| | SRISNAMEQVYLFEKIAESVGRKSLQLYPMAITSL | |
| | MYSFSRAGHVHGPLMFYGSQHLTKFAEHYNIVEL | |
| | SMVSRAHCLLEIKNDDLMHCIAREIVKRFPNVIPT | |
| | AADSPRVRRISQDSKDLEHVPQESEKCLILEAGTI | |
| | NLLWIMQGFASFYIFDGNIRNAIMAICNEMCMRIV | |
| | DLTPMLVSNFLHALATLRYRHETFLEILVRELEDP | |
| | RLGVKFNQDELKLCYEALNTFGVHGPIYKVSKM | |
| | ALKQAHEINGGDSTTTLRQVMAGELKTFQDDSEE | |
| | LTQELKIPPPPKKKVYIHVPEVIRKHLQVPNAQVT | |
| | HQYDFVTFQV | |
| BdWA1_I0231, | MDVFSILLVFSAFYVNAIAADDVKTFLFKKDVES | 23 |
| hypothetical protein | TVEIDANDDAVLVCPIASVLIIKKARWLPVTGGD | |
| | MRVKDGFSRTTRIGWLCNGLENCAFRPVAHLSKI | |
| | GDRYEFLGQPIETDIYKLTVTATCGNFMFKRPGR | |
| | REMLCIPTSAKPDIVLGCKDNEAIELSYVRVGGKS | |
| | KHQWRHRDYCAESIIKTAHPLCTGKKTCKIAHDV | |
| | FLKNAKECIPREFNVEYYCAAPHKNSFYDPLDAV | |
| | VVDGVSVATKYVLTAEDGARASAKTNAYQVLQ | |
| | VDSALWESDGATERRDRLELVKFLCDGRAECVFS | |
| | PTRSIIGPDERKCNDVVFGGMVKDTMSHFMLRAH | |
| | FSLVPFDPKKYDEKEYHHVTIKSTEKKTLECPVN | |
| | MSLTFYVALWGGKITDTSPLKGPKHFVEVDINGE | |
| | KHRYSEIINIVGTQCFGKSKCEIEPLKLKPPRHEKD | |
| | LKEFPTHEGVKKDDHQLELYYKCIDLQTLPSLVES | |
| | LISDGPRYPREFITPIQLSPDMRIVVMLDIYGPTVL | |
| | EVANALKLEIPVARTNEIKISWKDAKISQGIRLVK | |
| | DTRNYVFEFVIGAEDYIHMTVNSFDNDGSPMSIPV | |
| | EFEASKRILDFSRGIEDFVVATGEITNFRAFIKS | |

TABLE 4
B. duncani secreted antigens identified in the supernatant fraction: SEQ ID NOs: 24-
37

| Antigen ID | Annotated Sequence | SEQ ID NO: |
|---|---|---|
| BdWA1_II1370, | MVTREMYIMGYANVISADIKKFKETLANEITTREE | 24 |
| hypothetical protein | YEKLKADIASTRAIMGDLKSNVRVLTRLLNEVYIL | |
| | KTLMREEDVRALGDLSMEAKTLVAGKKELQIKL | |
| | ENEIREIKKKVDGLPLTKKILEENTEQLLEEISKIE | |
| | MHVKKSAEAIEKNIDECNDKITQTNVMAEEEHEA | |
| | LTNKLGITKIVLNGIKTGLLKLISISNRLKAATSGA | |
| | TREENKILKETILNRIKFILAEGDVVFKRLEKDMT | |
| | DIEKRIKRIPIEGTLPDLNTHGGYGTIETH | |
| BdWA1_I0253, | MDFLWLLGFAIIYRKFVVGVGPDEDSDYPEVDVK | 25 |
| hypothetical protein | SSKVNIGISATVDKFFDDMKLMEEDYKAYQDKIL | |
| | GALNTVRRRLEKAKHLEATDLSDLANIMLDVNK | |
| | ELTKMNSCVSRLAALRPYVEKSINLLHEDDKETA | |
| | KKRLLNFMNPDEMVNVLHMLFSEYKELNVELVH | |
| | VKKRGTAPSQSESLSKSKIESLSKSLSALNQEPGRI | |
| | LEPALAIYDKIMNGGTEILEEMNKLNLKIQGKQT | |
| | MSSMEYLYIVQNMLHAKEYVMESKQPLTSLGYL | |
| | GTALDQMQFTYSPTEKSHVENIKKEVGEILNGIKD | |
| | FQNKVKNSIDTVEKRVTTISVTDALPKERALEIISA | |
| | VPAYVQIFKKQMDELKGAVLNDINELDKQLDEK | |
| | KRIPSKEHEQMEAKIPILETQVQFFLEFIEAMKYFR | |
| | VTCEMIPKMMGVDEKKQFRRELILCDMALKNEK | |
| | QEYDFMIEQFKKVKERIVQTRPRASRFKRKHSGFS | |
| | TMEPSSLLLVLPVIVSLFY | |
| BdWA1_III2944, | MGNACCKSSPPAAVSDAKTADKPNFESLSTLSMK | 26 |
| phosphatidylinositol- | NDGSHRNQEKAPSAKASPRKNVEFKFTDTGYDA | |
| 4-phosphate 5- | TGAKKWDEKVISALLGGKPTNIEAAPGIDVGDGL | |
| kinase | VERGPVLLKDGSVYCGQWKGSVRHGRGQFFDV | |
| | DGTQYIGNFSKGVFEGAGELRTWTGDKYQGLFK | |
| | NGKYHGKGIFTQKNGDVYEGVFVDGMREGYGTE | |
| | RYKDGSVYMGEFKGGKRMGNGELKMADGVLYE | |
| | GEFNDEITGKGKMFWPTGECYVGSFLKGMKHGL | |
| | GETTWKTGPMKSQRGKYENGKMCGTFENVMRD | |
| | GKVVKGVYKDGILLQEITDTKPKVAPVVTQPPPV | |
| | KEQATPTPTPKAAASPTTSTPTRAAASASPTLSRA | |
| | PSTSSTPATATPPASTPTAAASTQAKPKAKSAKSS | |
| | SAAKKKTSSKSSAR | |
| BdWA1_I0708, | MADEEVTALVIDNGSGNVKAGVAGDDAPRCVFP | 27 |
| Actin | SIVGRPKNPALMVGMDEKDTYVGDEAQSKRGIL | |
| | TLKYPIEHGIVTNWEDMEKIWHHTFYNELRMAPE | |
| | EHPVLLTEAPMNPKANREKMTTIMFETHNVPAM | |
| | YVAIQAVLSLYSSGRTTGIVLDSGDGVTHTVPIYE | |
| | GYALPHAMMRLDLAGRDLTDFMQKILAERGFSF | |
| | TTTAEREIVRDIKEKLCYVALDFEEEMTNAESSSEI | |
| | EKSYELPDGNIITVGNERFRCPEVLFQPSFIGMEAA | |
| | GIHTTTFKSITKCDVDIRKDLYANVVLSGGTTMYE | |
| | GIGQRMTKELNALVPSTMKIKVVAPPERKYSVWI | |
| | GGSILSSLSTFQQMWITKEEFDESGPNIVHRKCF | |
| BdWA1_II2269, | MAIWKLFVFGICGAGKVLGYRLDDVLQGDGEDF | 28 |
| EF-hand | ALFGLGKEVIEARMDKLFSVIDLNNDGILDLEELA | |
| domain/EF-hand | AFHAKTFQTILDLQLNHEMELVDRNKDGFVDVEE | |
| domain pair/EF | LKVAFEREGTQDVDISTVEKGLQRRFVAADKDQ | |
| hand | DGKLNRQELGLLLNPGRDEELINIEIEEIMQTYDQ | |
| | NGDGLVSLEEYSHGRSDQEGVSAEFKPFDSNADG | |
| | FLSREEIRGVYVEENKNDLDSEHEDLFAITGKKPIT | |
| | REVWNANLNKIAHTSLTDHGEMLRFPEDYHMDL | |
| | GDIPRDRKDGAEERPSGEL | |
| BdWA1_II2320, | MCGNGIINNLVYLKMDSIRESTSFHNDFYYITCPF | 29 |
| Sodium: dicarboxylate | SIKGFLKEYWLSTITFFLTFAAVFIPSVFKLDLKDH | |
| symporter family | ADYVMLPANMFLRHIRGFVVLFMFFASAAKIRLF | |
| | LQRKVDSLKTRIMIKYLIAGLLSLLVTLGLAALIIP | |
| | LNTSLGGNTYFSPHSIKDYNPTTEFKNFINHLAVH | |
| | DLPLNMATTRVDFVNEFGKDQLHEGLGDGYNAP | |
| | GFVVYGLLFAFAIYTMDEHTDALCNIITAMHKCL | |
| | LSVYWILVVYSPFAFFLAGLVTFDELKVKGGLGIA | |
| | LMGYLYLTLAVLAVFVAWSFIVVPLLHFIRTARN | |
| | PYPTIIKLLPYLPTAFCSGSSVLSGEKTKDFLKKRG | |
| | FDPDDVENYLTFSTLVNFSGTTSGFTVCAILMLKL | |
| | FGKSLDWVTVLKIIVTGLLVGFTIVEYIQGYLFGII | |
| | FFLNNTMLPPGSIILLLQIDWLLDRFRIVSNVIDDA | |
| | LTIDEITNVKSKLSSCPFHKV | |
| BdWA1_III3035, | MGEGTNKTQYPFILGLHEEVVCAAKRLEICLLEKI | 30 |
| hypothetical protein | EQLGHVDDKGWKVCVDSTIEGKLLGNHITCLSLC | |
| | KVPNGSHGSEFLLAVGYKSGLIALARVDSQNEFHI | |
| | LSHNDQNKSSISQIELKYVGESTAAYNLMSLRVY | |
| | AFLDSGKIITFSWGDKIQTSIQSGEHVLGFAVNSQ | |
| | NTTIAILQNTGIVCRSLDTETILRTLENVQIVNANR | |
| | HYLSWHPYKNLLVFLDNKGISYTCHPKWDVYKF | |
| | SQNSQHQELIRHVQFGICGDFVLLLTASFDKIIIWD | |
| | FETESILYSHKGCDMVACGLIHLSNKRVRLAVFA | |
| | NFASNEFWRCKRLIMSGDELMEEAKSELPQSHKT | |
| | RRLKRHITELDDYHIRKMVDQEAVDEDNEEQDEE | |
| | YKDEETYRDILDSYDNLDKDEMTHFDQSRVHVM | |
| | HEISKLRKKVASLDNRVAERTTLVPGSCPAPDDA | |
| | AVQWVLFWDEVGQITKQLISDVWCLHVHVFSGP | |
| | LAGYKRKPDRYNCHTAALNQKYVVTGSEINVDG | |
| | GHVHGILTFLDFENNTQWDRRFFNEYISAVAVGD | |
| | SFVAAITADGILYILSLARSLMGVFQLKGSPIAIAA | |
| | RGNVLATITEATTLTNVSSFGHVRMFWVNGLRGL | |
| | AKNPASRIVDLYSDSLVLGPDKAISWISFSSQCTL | |
| | WITDTSGQLMALLPAIESCYKVGGYSLEWIPFMN | |
| | LENLVNESSDYEPSHTVFPLYVSDMKLNYILLKR | |
| | GQSYPTCSQPLNFMGYTLKKACLRIDGCVGAYLP | |
| | FQKFNGLVTKDPQLREVIGSDIISDVASIPWQQYD | |
| | EMRHIQSLQACQNEYILKVFQNYNFWMQNSQGIS | |
| | DDAISAFGNAERVHDKWTLRILRKTKDSKQDSGV | |
| | LLDALWMLRFPKCLEAAFSILQNQLDAKQRQVL | |
| | QEASLLLENVTPFENNVQPLDAQSQPTATVSIPKQ | |
| | DLDPIPKKHAPLIQGPSNRLQEFVPLGNEPEESGPL | |
| | FKNILE | |
| BdWA1_I0924, | MVDFEPESCRKHVDKNTRIYVDGAFDLLHWGHL | 31 |
| ethanolamine- | NALRQSYKLGGELIVGINGDVETFHAKGISPIYNQ | |
| phosphate | DERAELVKGCRWVNEVMVGTPYEVNLDFLVNIA | |
| cytidylyltransferase | KCDYIAHGDDIAIGASGKDAYDEPKKAGKFIFFRR | |
| | SLGVSTSTTVGRLIDALESDHFSHLSKNSENKLQY | |
| | GDFEKALQENEEQMINEGIIDDALFSGNNKHDNK | |
| | NKKSTDVFPYPRFRLSTSLLGEFITPKPKPKGGKII | |
| | YVDGSFDVFHVGHLRLLKRAREMGDYLIVGIYD | |
| | DQTVRTLKGTPFPFSSLMNRALTILGMRYTDDVV | |
| | LGAPYVPSRTYLENLGITTVVTGKQHDSKMINRD | |
| | FDPYREARDMDILVEIDSGSDITTSDIIARVSSRMD | |
| | QITANIRKRCAIEKTRKNIVCSL | |
| BdWA1_III2663, | MLNNANTVKEVGFRKSVIYQPNVFKDADGVIKIK | 32 |
| MAC/Perforin | TNNKNIWIRQNSKCWEFTSKTPITSVDDYYKELLS | |
| domain containing | DFNTDDSTSITLYNANVDLAKRGFNNFHEGYTKI | |
| protein | KKLYCSILEAGLMIPYSGMLSGDFMYAVVSLPDK | |
| | LDITYCPIDNYMENPTKSECENINRWMEFFKMYG | |
| | THVSTHIITGGKIFHEYKTLNITEYRKKSKFRQSTN | |
| | IFEVTNINFDSAGQKSTKNTIVFGGNYVKGMESEA | |
| | RHFYNEWSKTLESRSLPIKVTVKPLSIFMSYRNDI | |
| | YKEALKFYRDVALLTTVGIQYSTKIDELLRESTTV | |
| | VSDDGMAHCPTNQIVLAGFIMSKNPKVPIVNCEQ | |
| | GKILCSNGTDKPASVYIICVKELNDIITTSTSMENV | |
| | HICPNGNVTALGFAFRKQSESDDWTVVIPRIGKQ | |
| | QMINYTKGLNMSWLLCVPIEIMFWHMEMIIDSAP | |
| | KDDSRTVSCKTGWTILKGFKLMFLKKEDTTRVVL | |
| | EECISHQQHCILNCNEECTKMYGTILCKKQHD | |
| BdWA1_II2261, | MSTNGTVRWRTSSRSQLRDRRKNALTRRLRFEV | 33 |
| RNA polymerase | GRLDTEAFETRTKYLREIFLDVDKRNSHDWTALE | |
| Rpb1, domain | KKILLLDWCQNPQKFEKWKPAATEDMQSRFFAN | |
| containing protein | LIFNYSHLAMINSGMCIVIVRLVPLVVNIKAPYQC | |
| | VILLVRDNVNGMVIDRLKVGLIWRGKDTNTDVL | |
| | GKSTRIFTPPLSTDFQFLSHGIGYNFKSLLPQTEFEP | |
| | HVACSDCISRELESLCTLETPDIVLESPALGMEVTS | |
| | SFVNGHKVYLVVTRGVDGIVRIHRVQELIQTSIDII | |
| | RTWEQLRLKHLKRTMQSQQYAACTGCLVHKEEL | |
| | QQLYHLKQCARSFAPECGPMQFVGLEMIDVARP | |
| | RTCATKREACGLPLALGYTSVRNEQAVIPSLLMA | |
| | MASNTVCIWSIYSYLKEWNLNPLGPIKVMTYPDG | |
| | TLPTDISSTVSIAASLEALERLEFIHAPKFYTTDDR | |
| | GHLSLWSLGSTGPEAQVTLDQNALCSCAVNRSYP | |
| | HIVAVGLDVGKIKIYNTLTQGTTCIAQMEHPLLTK | |
| | VQTLQSFLPDNVYDYQRWYHPVVKLEWISDVFIL | |
| | AQYSEPIFTTESTNASAVAIWNVVKDIFDRDDALV | |
| | CNKHWSLNSCNLYNTWHLASKLVCLYGGHFGCI | |
| | CGVLSSDAKWSAEQGLLAVTIDSTGQLHVFKPGI | |
| | WTWGDCDDPMAIARLTGDCEFYHSILARVQEQH | |
| | RRLSVPVMEIDNSVDMGQGTRLRKTRAKFSTYIQ | |
| | DAQTQLESVETASGLEEINSLEKLPMFCKKTLRLN | |
| | SESEKYLKLVHRLVAEREHAEFLLSQGTPGKTGK | |
| | RKCPLGAPNIKQEPLELIEAPQGPSSEAPVKSKNE | |
| | YQNDAMEPESFNFSSVLTKFDTDVSMEPETELVE | |
| | TALPVVQPTKKRLVEKGLEACCIEGVQFDIMGDV | |
| | EIARCAELEVQKRELYYYLSSMPFPQGVLDLRLG | |
| | CNRNDTKCETCGRALMECVGHWGYINLQLPVFH | |
| | VGFFKYTIQLLYCICKRCSSLLLPFDTVAQLRDAR | |
| | LRRSDDPLARAVIFKRILSSCRKVTKCPACGARQG | |
| | VIRRIVKPTMDQFMKLRHVVKYKEGGKIVIVEDE | |
| | LNPLSVLRLFEAIDPVHARILNIMDPQRLIISNLPVP | |
| | PACIRPSVSLQGQGTTEDDLTCILSDIVELNNVMA | |
| | TQMQQGFQTNQVIGNWQFLQLQCTRLINADAPA | |
| | VSQLLAAKHISKPGRGICQRLKGKEGRFRGNLSG | |
| | KRVDFSARTVISPDPNIGIDEIVIPEYIARRLTFPEK | |
| | VTSANLGVLQKAVLNGVSKWPGACYVMKRDGV | |
| | KCTLRFANPKQVAESLQIGDIVERHLWNGDVVLF | |
| | NRQPSLHRMSIMAHKARVMPGSTFRFNECVCNP | |
| | YNADFDGDEMNLHMPQTYEARAEALHLMGVLQ | |
| | NITTPRNGDPLIAATQDFLSASYLLTSKDRFLSHQ | |
| | EFCQLLCYVGDGILHAQVPAPAIVYPTFLWTGKQ | |
| | VYTAILRQIGAVVNLECREREFQHPEPNLFGRMQ | |
| | PNFMCIKDGHVIISNSELLCGALAKKTLGASKDGL | |
| | FYQLLRRHGPHKAADVMLKVSKLTSRWVSDFG | |
| | MTIGLDDTTPSPMLLATKQQLLSDGYAKVELAIA | |
| | NAANIEPFPGCTRKETLELQVKGILDDLRNQAGK | |
| | ACNKSLSANNKPMIMFNSGAKGALINIAQMIACV | |
| | GQQNVMGQRIHHGFIGRTLPHFEVGCIDAKSRGF | |
| | VSNSFFSGLDPAEFWFHTMSGREGLIDTAVKTSET | |
| | GYMQRRLMKALEDLAICYDYTVRTSDGQIVQFIY | |
| | GDDGLGASGAHATTPKALQETLAHVLTLSRFQRT | |
| | PTILNSRAPGPNSKGDLRLPLPSGDGDDENFECGI | |
| | VPKCKLAMPPKVKAKASARASWRTSQVGTPLDE | |
| | ATSAQINRNMYAHWVVHVHLDPEHAKAPLFGDE | |
| | VLDWVALLEPLCREPLPRCMQESVTSMDHTSEPR | |
| | IHASSMEIDKRVNGCSMKATNTGPRFSAVLLDTIR | |
| | QWAARLQTLDPEIQREYESSGMDLVQFRAFKIPP | |
| | QDRISVLQIYEFLRCAWSDYLHGICEPGEAIGALG | |
| | AQSIGEPGTQMTLKTFHFAGVASMNVTLGVPRIK | |
| | EIINAASVIQTPIIEVPLAKKDSYDFAKSVRAKIER | |
| | HTLEQVVHSIKQVYTPGATLLLVSLDSQYIQQNM | |
| | LQLDASSVCAVIRNSNLITKFKLSKHNIEAPQKWL | |
| | VSIRLVASEALLFQLNALVAALGQLVVCGVKDIK | |
| | RCVVKREQKDVINSLTPGATFEYALAVEGYGLQQ | |
| | VLGIFGVDAHRVISNHVAEVAKVLGIEAARLVIVT | |
| | EIKKSMDAYGIDIDGRYMKLLGDVMTFRGEVVGI | |
| | NRFGIQKMRASSLMLASFEETNEHLFQAAVHGRR | |
| | DPIKGVSECIIMGKHIALGTGAFDLLYRE | |
| BdWA1_III3073, | MANGGLYVFPLLLCMHSVYALQCPHAHRACFLK | 34 |
| PP-loop family | YNGIYTSKANKLGVLDVVNVEDNALDLEDVVPL | |
| | QEVYKFFNNYLMPFYHKRVSQNYGTNVPIIISCSG | |
| | GVDSMALLHTFGLIKENSHTFIKKHPINYIDGSNA | |
| | VIAETASNVLKYIFENVNVIYFDHKVRSDTKVDIEI | |
| | IENACKKYNFNFNVQELDCNDSYFSNLEGGFQAN | |
| | SRKWRRRELKKYVMELHSSKMLSNEGKNKIGIR | |
| | NGSYNSITTNKQDGLNSNSIGIVFMGHHANDNIET | |
| | FFMKFIRGTHLLQMSEINDQSFLDSKDRIPLIRPFIH | |
| | LPKNALHQFMQDFRFQYNEDSTNVLFDYGRNQF | |
| | RKLVMPNLIEYIMSINKCQNDKAIDLLDKRIHVLS | |
| | KQASNFRNDIEFQIQMFKVYLKSKYGDLLPIKKK | |
| | RYMNRMGEIYSDFYKRLYFNRYNTAIHSNIQELK | |
| | YFHDLNIPIMDLFFVDEWLILESKLLREEVLYDFFS | |
| | HHFRKPLFYNAFTKIVDRLEVNFSEGSIKQYCLSK | |
| | DVSMSHQGSLLKVKHRPEKNEKVLIFKDDLCSFS | |
| | VLDNLQLFVEKAEGYTRNVFHLLFKVPMYATTES | |
| | IDFDIRLFRNDDVLPSKILWNREAGSLLTHMKIKHI | |
| | IKDYIPVIAMAGTNKIVGFYGFNIIPPYHCRGRQEV | |
| | TYSFVDVAHGSTEYREYSIRVTR | |
| BdWA1_II2013, 5′- | MGFFIQSQLLSTLWILIVIPQNVQCLRNNSRNNSL | 35 |
| 3′ exonuclease | AFASGYAHSNVRNSLDSLNGLPNLINRKEPTCQD | |
| | GFKLFGVPRMYGWMIENLGKINQSFDTCDFYED | |
| | VDYFYIDMNAVIHSATHGNMSPIVEIEDEQRMRRI | |
| | TSTLLKLFHMIKPKKVMYLAVDGVCPSAKINQQR | |
| | TRRFRLAKKVEDLTARVQDICEMKPIEDYNIDKLP | |
| | CGSYDNVTFNPNYISPGTEFMQMFDSEIKNWLAI | |
| | KTLEKQWGDCLVIYDGANIPGEGEQKIYEFMRKL | |
| | NESKVKSRNKNHLVYGLDADIMMLSLLTKMPNV | |
| | CVLREKRDYTPHILSKIKPEPYFTPETGIVHYHAN | |
| | DYIDLEAKHFDVVSMMDIRRALYNRCIKYVGLK | |
| | YDLANIPFLNQENVPNRLADDFVLLSFLAGNDFLP | |
| | HLPTVDLEFHTFSDMLNSYFFMLPRLHGFITRGYR | |
| | IHMGRLQKLFRVLQRQEVHVFKEKAQHESVPEY | |
| | RDVTKYAEHYYRVKCNINYKNRNQVTRMCQEYI | |
| | KGLVWNLYYYYKGCPSWNWCYKYHYAPLVSDL | |
| | SQTSGVFVSFKRGRPIKPLEHLLAVSPPNGNELLP | |
| | EQYRKLSASPNGELAEFFPTDYEICEDGKVNEWE | |
| | HVVKLPFLNTNKLVTHAEQANQHLKYNSLSKNK | |
| | LGRVNVYKCRPHNVNKSGDNRLIFDDLTKKGLIV | |
| | RDGMHYGATFSVYEERPGLAHSSCLIFARHGQTPI | |
| | MIKDLVRWTRISQAANKRVLIITILYKTMNTSGWS | |
| | SDFSSLSNSDKITGKDRINEQESISVPSGLHCGPAG | |
| | RRTLIKWIEENYRVRKGMDDTNPGVVACRLWQD | |
| | SQGFSDPVSKAAIDLLRLQGIPANVTYKTLFEQHL | |
| | SKMLSSLINQSRNSQARLYSIAEKMIGMFQVAEIQ | |
| | PLICDVLDQLDEIPQFALPKLLDDAASANNFYKIC | |
| | NDGLKRKIWATSAVRLYEEMLPLFQEAVLCIQLG | |
| | VLEPRMHHASFIQSCRERYEIVIEQICQMIGDCKS | |
| | QASRQVFRLTMRIIKVLYVKSILGDKGDFQIYFTP | |
| | HDHLDTALKLNWRDLAARSVGICFKSLQLEELDE | |
| | AHDSFSSTDHSSFCALAHSIISRHQDMHKITSSDM | |
| | QDLESFYTLWSLVDTVIASPCMSLDNEQIASVLN | |
| | VVLGDLRIRDEIDLFDASFVLGHFEFLIKISKYIVT | |
| | RSININEQLPEDQDTLGQWFSLASLGLSCGYIGAC | |
| | QFLFRAQWNNDTCIWHFPTETEPGKPISLSKLLGY | |
| | TFPYNRSDQMCLDTLPPFFYKIVHGQASPHLSETS | |
| | PVRNPYEANKASLRNLLCLYLPMVDVWKFKRA | |
| | MMDLKAFEQNGYGTLLNSFKRQTFQTEKLLFYM | |
| | QDMAIRDFESDDYDWTIARKLYQNLERLQALKL | |
| | ATPPHGTVMDPKSPQPHVDDGKRLMGTILIQGDI | |
| | TCGFIRMMGVSMVEASDEMARRLENYWKNLLQ | |
| | PYMCCMQFRLLRMLFSNWTKISSFSLDVLCKMV | |
| | KGYPIVAFLPLFTKAPNCDISTALGRLSASLQGTLP | |
| | NHSSKGWHRIVFTMYNKHS | |
| BdWA1_I0563, | MKLQINSFNHIFFIKQNIRTNLFKLSQKPHPFNLKF | 36 |
| hypothetical protein | FRQYSHHEIFTSIKLASNKSEMASVTQILLNRSNY | |
| | KGVVTLDNSTVVSANDIVLYSEFNSKISFSLTLDT | |
| | LKKLTKLQTPCKVVWNYVIDSILSDSSHFTPNEIA | |
| | KILVYILNIKFNDIFIKSKIELASETLIEKLNGSLSLM | |
| | VGFSVIDIKRAGLRNISDTIRHDAEFDKLDSMDFEI | |
| | IWKLWYTLSKQRGNERIRGLLFEYLERGKDKVYK | |
| | FDLPKVIRSMDIILSNSKSLDLLELKFLEKLFKRFL | |
| | NLYNSDNIPTFGGSSNIYMTTIQAKKALYILGNICK | |
| | RNPKIFENIVKHECAMLSLVNFLKQVKEITSRSLE | |
| | VHCRLYNHLKNINVENTNMKHNVRIEAIKRSSNV | |
| | HAKMFVYCVEFLNNFDLITKANYNNASSYSKATS | |
| | DNKRSTDLDHTIFDICTNLLRCTAQSSFLIHNVTM | |
| | YCRSIYQLLVLALYNTCTGINESFVKAALSSISNIQ | |
| | ATTLQMDRDNWDISSVIRNLSYIDKGISVLADISY | |
| | SSIILEPIQNANAEIVSKCSSKYEEVLMGGEILQLD | |
| | KLVYCTLLNKYNIIDGTMLDVLLQFIKANELTSRE | |
| | LCMFCDLLLKTREMLMDDCDKRNNVLAKLLPLA | |
| | LELLKSIDFDFNKRTLKDLLLILECLGAFGFSCAD | |
| | DLSKRILEHGAKLTISQLMKLYSIVNDDYKRRIRL | |
| | LLPGILKQRGKINITAILKVLNVLDIDYNLLETLEH | |
| | NLGDDLYLVDKAYFARKRLLHQRNSSNPRKKTK | |
| | VDYNNSGSHHMFIIDSSVAKAFIKCNKGTHSSSKL | |
| | YKILVSKYQSSSSSMAL | |
| BdWA1_II2256, | MQGGWLLVCIVGFLACFGASTKPNDKKATNEEL | 37 |
| hypothetical protein | TCPAVNDLQGTPIDLQYTFDRLDMTSLSRLLAIQN | |
| | MYSKRNPRNIRIPKFWNTDVAHGIWSRFRIYGTN | |
| | LVYKTSRGDGSCLFWSVSDSLRLGGFTIKKIKENL | |
| | DHYGTKNRGIYEAIMSLPNDDEYLTMQDIQRIATI | |
| | GFVGFDPDVEASVKQWDREPILSHLETVKSLKTA | |
| | YMGDVIPFDLGCHRFTSFRAVDDFYNGIANDATC | |
| | IKAGRDLFNHTIKYLASGAWGYEIDIYAIETALNL | |
| | KIVLISYNTGSLVCYYWSEDYVPQSMILLYYHDS | |
| | RHFDVAGLVDMFKVPNVRNPRAKVLTSFHIREM | |
| | PMALAIILKDDCRITKRNERFSFGNEDLTHLY | |

TABLE 5
B. duncani secreted antigens identified in the hemolysate fraction: SEQ ID NOs: 38-
46

| Antigen ID | Annotated Sequence | SEQ ID NO: |
|---|---|---|
| BdWA1_II2291, | MQMFNRFLKASVALLAVASFGIQYIFAKGSNSGK | 38 |
| heat shock protein | IEGPIIGIDLGTTYSCVGIYKNGRVEIIANEMGNRIT | |
| 70 precursor | PSYVSFVEGTQKVGEAAKSEATINTESTVFDVKR | |
| | LIGRKFTDRDVQEDMKLLPYKIINKSTRPYISLHD | |
| | GKEQRTFAPEEISAMVLKKMKQVAESYLGKEVK | |
| | KAIITVPAYFNDSQRQSTKDAGAIAGLDVVRIINEP | |
| | TAAAIAYGLDKANAESNILVYDLGGGTFDVSVLT | |
| | LDSGVFEVIATGGDTHLGGEDFDRRVMDHFIDIF | |
| | KKKHKVNIRDNKQSLQKLRKEVEAAKRTLSSTTE | |
| | VLVEVENLINGIDFSEKLTRAKFESLNAELFEKTL | |
| | ATVKKVVEDADIPIRDINQVVLVGGSTRIPRIREMI | |
| | KEYFGKEPDYGINPDEAVAFGAAMQGGILSGESS | |
| | DNLLLLDVCPLSLGIETLGEVMSVIIPRNTMIPAHK | |
| | SQVFSTSVDNQPMVTIKVYQGERKLTKDNVILGK | |
| | FDLSGIPPAPRGVPQIEVTFDIDTNGILSVSAEEKG | |
| | SGNKHNIVITPDKGRLSPEEIERMIKDAEMNAEKD | |
| | KEVFNRVQARQALEGYIDSMTKTINDDKTGKKLE | |
| | DDEKEKIRDALDEGTKWLASNPEVGADEISAKQH | |
| | EIEAICNPIISKLYGSGEDSDDSGYSDEL | |
| BdWA1_II1496, | MAIPDNNNNTQSNGFDTLESNYDEVVDSFEALKL | 39 |
| eukaryotic initiation | NEDLLRGIYSYGFERPSAIQQRGIKPIIENYDTIGQ | |
| factor 4A-3 (eIF4A- | AQSGTGKTATFSIAALQIINYNIMSCQTLILAPTRE | |
| 3) | LAQQIQKVVLALGDYLKVQCHACVGGTVVRDDI | |
| | HKLKAGVHMVVGTPGRVYDMIDKKALLTDKIRL | |
| | FILDEADEMLSRGFKGQIHEVFKRMPPDVQVALF | |
| | SATMPNEILELTTKFMRSPKLILVKKDELTLEGIK | |
| | QFFVMIDKEDYKFDTLCDLYESVTITQAIIYCNTR | |
| | RKVDMLTNKMQEKDFTVSSMHGDMGQKERDLI | |
| | MREFRSGSTRVLITTDLLARGIDVQQVSLVINYDL | |
| | PMSPDNYIHRIGRSGRFGRKGVAINFLTPLDMDA | |
| | MKSIENYYNTQIEEMPADIAAYM | |
| BdWA1_II2200, | MSKKLKTKGPENINHSLQLVMKSGKVCLGFKSTR | 40 |
| ribosomal protein | AALRSGKAWMIILSNNIPALRRSEIEYYAMLAKCS | |
| L7Ae containing | VYRYSGDNNDLGTACGKYFRVGCMAVLDAGDS | |
| protein | DILRNIE | |
| BdWA1_I0250, | MTSVNSDVDISEVSQMSDADIRVRINLIDSEIKILR | 41 |
| 26S protease | SEHTRLKSRQKTLQDRIKDNLEKIQLNKQLPYLV | |
| regulatory subunit | ANVVELLDFTSDDEQDDGLTPSPSQKKSKSLVIKT | |
| 6A | STRQTIFLPVIGLIPASELHPGELVGVNKDSYLVLD | |
| | KLPPEYDNRVKAMEVCEKPIEDYSDVGGLDKQIQ | |
| | ELVEAIVLPITHQERFKKIGIKPPKGVLMHGPPGTG | |
| | KTLLARACAAQTKATFLKLAGPQLVQMFIGDGA | |
| | KMVRDAFSLAKEKAPTIIFIDEIDAIGTKRFDSELS | |
| | GDREVQRTMLELLNQLDGFASDDRVKVIAATNR | |
| | PDTLDPALLRSGRLDRKIELPHPNEQARCHILQIHS | |
| | RRMNVNPDVNFKELARSTDDFNGAQLKAVCIEA | |
| | GMVALRRDASELEHEDFVEGISMVQAKKKNTLN | |
| | YLN | |
| BdWA1_II1994, | MAGHAALILNFGSCFSGVLVRVLRDVGINCVLES | 42 |
| GMP synthase | AEKALDALNTNSTVKVAILCGGLDSVYDETSLTV | |
| | PEEFIKACEEKNVKILAISHAFYALCKTLGARLMN | |
| | GKGNDYIIDTVTVQRPMVLFNNVGKHFKAKINPI | |
| | NGVEVLPAGFESLATFGNGHYAAIGDEKRGIFGV | |
| | AFHPESDDTENGLVILKNFCLEQGACPIEWSMEQ | |
| | YLKDELARCIAQCGDTKVVVAGLSGGVDSTVCA | |
| | AIVHKAIGNRFHGVMINTGLMRLDETKKCAERLK | |
| | KEIPGIQLHIRESADVFFGELKGILDPEQKRKIIGKV | |
| | YIDEFERAIKDLGFDKSNCLLLQGTIYPDILESELN | |
| | RRNQMPIKSHHNVGGLPKDLALELIEPVRLLFKEE | |
| | VRKLGRLLGLSQESCERQPFPGPGLGVRVIGELNP | |
| | RNLDLVRRADAWNQILDARGYRSKISQSGCILL | |
| | ADVHNTGIRNSGRTYGHAVIIRIIITTDFVTAQWA | |
| | RIIDTDCLAEISKTITDTVPEITRVCYDITDKPPACIE | |
| | WE | |
| BdWA1_III2590, | MAEEMPQFKLLLVGDGGVGKTTLVKRHLTGEFE | 43 |
| GTP-binding | KKYIPTLGVEVHPLKFRTNCGGIQFNAWDTAGQE | |
| nuclear protein Ran | KYGGLRDGYYIKGECAIIMFDVTSRITYRNVPNW | |
| | HRDIVRVCENIPMVLVGNKADVKERQVKAGHIQF | |
| | HRKRNLQYYDLSARSNFNFERPFLWLSRRLLNQP | |
| | QLVFVGECAKAPEIQIDPLLAQQSERDLEAAARV | |
| | AIDDDGDL | |
| BdWA1_I0810, | MADRFTGRNNREAVVAYPGWFSETQKQCLRAC | 44 |
| Hsp70 protein | VTASGLSCLRVISHVHAMAMDYGVYRVKQLNDE | |
| | TPTRVALVMIGHCHASAAIVDFYASHCSILSQVSR | |
| | RNLGGRNLDMMLMKYMATEFSKKYHCDPLENN | |
| | KTRLKVEAVAVKTRRVLSANAESSYSAECLMED | |
| | NDMSGHITRTQFEEMCNAEFIPQLIEMLKECIEAS | |
| | RTDLDSIFSVEIAGGSSRIPCIQQAISSIFNKVPSRTL | |
| | NADECIARGCVLEAAIKSNHYRVREYKTRLTLPR | |
| | SLTLGYFNGQEPMLLEAIAAGTPLGDPIRVTLQAQ | |
| | APVCVRVALGDALDPRSQDALGTLDIARHISQEA | |
| | QPAPVTTNDGAAIQTDEQDAEIQSESSPSGGISVTL | |
| | GFDDCGQFVASPECCEYRWLPATILDIARLEAAEL | |
| | EARGRDLKENSRLQALNDFETLLYTVRDKMQSS | |
| | HRDFIDPQMIPAYESELDHWREWLYENSGASQET | |
| | LQEGIDKVSSEWKRIDKYFKEHQNKLENLEPFLQ | |
| | RLQERYNFCCEDNNPNWHGATPEERLNFAQELM | |
| | DLDSRVRQMHQDESQRPRHMEPLFTMQQIQGEM | |
| | QKLLVSISEFCQAKAAKAPAQEPPEQQPKEQQE | |
| BdWA1_II1634, | MDAGGKIGGKIGGKVGGMGKGGKGKTGSGKGK | 45 |
| Core histone | KAPMSRAARAGLQFPVGRVHRMLKSRISADGRV | |
| H2A/H2B/H3/H4 | GSTAAVYASAILEYLTAEVLELAGNASKDLKVKR | |
| | ITPRHLQLAIRGDEELDTLIKATIAGGGVIPHIHKA | |
| | LMNKGPAQVLVKPPKRI | |
| BdWA1_II1773, | MGIVTASIAPLHIGDDLYTRMKTLEKKLEICEIQE | 46 |
| 26S protease | NYVREEYRNLKLELIRAREEIKRIQSVPLVIGQFLD | |
| regulatory subunit 4 | MIDKNYGIVSSTAGSNYYVRILSTINRELLTPNSSV | |
| | ALHRHSHSVVDLLPPEADSSIQLMQVSEKPDVTY | |
| | ADIGGLDIQKQEIREAVELPLTCPELYRQIGIDPPV | |
| | GVLLYGPPGTGKTMLAKAVANQTDAKFIRVVGS | |
| | EFVQKYLGEGPRMVRDIFRLARENAPAILFIDEVD | |
| | AIATKRFDAQTGADREVQRILLELLNQMDGFDQN | |
| | ATVKVIMATNRADTLDPALLRPGRLDRKIEFPLPD | |
| | RRQRRLIFQTITSNMNLAADVDLETFVARPEKVS | |
| | AADIAAICQEAGIQAIRKNRYVVTTKDFERGWKR | |
| | HIRKHERDYGFYGV | |

### Example 5. Babesia microti Secreted Antigens: SEQ ID NOs: 47-63

TABLE 6
B. microti secreted antigens identified in the supernatant fraction: SEQ ID NOs: 47-
54

| Antigen ID | Annotated Sequence | SEQ ID NO: |
|---|---|---|
| BmR1_04g08775 | MSSQETFEFNADISQLLSLIINAFYSNKEIFLRELIS | 47 |
| | NASDALEKIRYELLRDGTKVSDESEFSIKISADKS | |
| | NNTLTIEDSGIGMTKADLINNLGTIAKSGTKAFME | |
| | AMQSGCDMSMIGQFGVGFYSAYLVAEKVTVVSK | |
| | HNSDEQYIWESSASGVFTITKDETTEKMKRGTRLI | |
| | LQLKEDQTEYLEERRLKELVKKHSEFISFPIHLLCE | |
| | KTKEEEVTASDDEGDKKEDDKKEDDEKEDDKKG | |
| | EDEKVEDVSEDKKKTKKVSTVTKEWEVLNKQKP | |
| | IWMRQPNEVTNEEYANFYKNLTNDWEDHLAVK | |
| | HFSVEGQLEFRAILFIPKRAPFDMFENRKKKNNIK | |
| | LYVRRVFIMDDCEELIPEWLSFVKGVVDSEDLPL | |
| | NISRETLQQNKILKVIRKNLVKKCLELFSELTEKK | |
| | DDFKKFYEQFNKNLKLGIHEDSANRNKISELLRFE | |
| | TTKSGDEAISLREYVDRMKPNQKYIYYITGESIQA | |
| | VSNAPFLEKLKDKNIEVIYMTDPIDEYAVQQIKEF | |
| | DGKKLRCCTKEGLDIDDEKDEEEEKRFEQVKQE | |
| | MEPLCKTIKEVLHDKVEKVTCGKRFTTSPLALVT | |
| | SEFGWSANMERIMRAQALRNSSITSYMVSKKTM | |
| | EINPYHSIMKALKERVAADKSDKTVKDLIWLLYE | |
| | SALLISGFNLEEPTQFGNRIFRMIKLGLALEDDQPD | |
| | DTDLPPLDEGVAVDGGDSKMEEVD | |
| BmR1_03g03490 | MPKEKTHINLVVIGHVDSGKSTTTGHLIYKLGGID | 48 |
| | KRTIEKFEKDSSEMGKSSFKYAWVLDKLKSERER | |
| | GITIDITLWKFETQKYEYTVIDAPGHRDFIKNMITG | |
| | TSQADVAMLVVPAESGGFEAAFSKEGQTREHAL | |
| | LAFTLGVKQMIVAINKMDSCQYKEDRYMEIFKEV | |
| | QQYLKKVGYKVESVPFVAISGFHGDNMVEKSTN | |
| | MPWYKGKTLVEALDQMEPPKRPVEKPLRLPLQS | |
| | VYKIGGIGTVPVGRVETGQLKAGMIITFAPTGLTT | |
| | ECKSVEMHHEVVEVASPGDNVGFNVKNVSVKDI | |
| | KRGNVASDSKNDPAKEATSFSAQVIVLNHPGTIK | |
| | AGYSPVVDCHTAHIACKFESLDTRIDKRTGKTLEE | |
| | NPKTIKNGDAAMVTMKPNKPMVVETFTDYAPLG | |
| | RFAVRDMRQTVAVGIIKAVEKKDPSSAKVTKSAV | |
| | KAGKK | |
| BmR1_04g05965 | MTKIISACGREVLDSRGNPTVECEVTTEGGKFRAI | 49 |
| | VPSGASTGIYEALELRDGDKTRYLGKGVQNAIKN | |
| | MHNIICPGIQGFLCTEQEKLDNHMVKVLDGTQNE | |
| | WGFSKSKLGANTILAVSMGAARAGAAAKGIPLY | |
| | EHLAQLSGKPTDKFIMPVPCLNVINGGSHAGNAL | |
| | AFQEFMILPTVADNFSNALRMGVEVYHTLKKVIN | |
| | KKYGQDATNVGDEGGFAPNISTPQEALDLLVEAI | |
| | AAAGYTGKIKIAMDVAASEFYQKDVKMYNLTFK | |
| | SSSPDIKTSDQLVELYKELVNKYPIVSIEDPFDQDD | |
| | WEAYAKLTAAIGDKIQIVGDDLLVTNPKRIEAAIQ | |
| | KKACNALLLKVNQIGSVTESIQACKISQENGWGV | |
| | MVSHRSGETEDVFISDLVVALGTGQIKTGAPCRSE | |
| | RNAKYNQLLRIEQELGERATYSKVFNK | |
| BMR1_03g01010 | MQIFVKTLTGKTITLEVEPSDTIENVKAKIQDKEGI | 50 |
| | PPDQQRLIFAGKQLEDGRTLSDYNIQKESTLHLVL | |
| | RLRGGD | |
| BMR1_03g01340 | MQIFVKTLTGKTITLEVEPSDTIENVKAKIQDKEGI | 51 |
| | PPDQQRLIFAGKQLEDGRTLSDYNIQKESTLHLVL | |
| | RLRGGMQIFVKTLTGKTITLEVEPSDTIENVKAKI | |
| | QDKEGIPPDQQRLIFAGKQLEDGRTLSDYNIQKES | |
| | TLHLVLRLRGGMQIFVKTLTGKTITLEVEPSDTIE | |
| | NVKAKIQDKEGIPPDQQRLIFAGKQLEDGRTLSDY | |
| | NIQKESTLHLVLRLRGGVIDPSLALLAQKYNCNK | |
| | MVCRKCYARLPPRATNCRKKRCGHCNDLRPKKK | |
| | IKGN | |
| BMR1_03g02390 | MGDDDNAALVVDNGSGNVKAGIAGDDAPRCVF | 52 |
| | PSIVGRPKNPALMIGMDEKEVYVGDEAQSKRGIL | |
| | TLKYPIEHGIVTNWDDMEKIWHHTFYNELRVNPE | |
| | EHSVLLTEAPLNPKTNREKMATIMFETHNVPAMY | |
| | VAIQAVLSLYSSGRTTGIVLDSGDGVSHTVPIYEG | |
| | YAMPSAIMRLDLAGRDLTEYMQKILVERGFSFTT | |
| | SAEKEIVRDIKEKLCYIALDFDEEMQAAETSSDLE | |
| | KSYELPDGNIITVGNERFRCAEVLFQPSFIGKECH | |
| | GLHKTTFDSIIKCDVDIRRDLYSNVVLSGGTTMLQ | |
| | GIGERLTKELSCLAPSTMKIKVVAPPERKYSVWIG | |
| | GSILSSLSTFQQMWITKDEFDESGPVIVHRKCF | |
| BMR1_02g03540 | MHNHSYYTILLTIALIHTTGCHGFLHRNIKFILHSM | 53 |
| | TVYGKPKRLHKTPPWAHLFEEKVEPSPLGEPWAK | |
| | LSKDVANGERRVRLTVKKSHLQIYAAVVDDYKN | |
| | QVICIASSNLPVLADVLGTVPTKDPTVRRNKGNN | |
| | VKAAYEVGKHIGRLALAKGVAKVYFDRAGYKY | |
| | HGRVEAVAIGARKVGLQL | |
| BmR1_04g09800 | MYQIDRMIDKHKDPIDPLQTILSVKGTMKCKLSE | 54 |
| | MLTRHSTEDRHSLSLVADLKRESPTHTDSRSGVR | |
| | LSFLDAGEVVVTMANTGFDVVLVNTDDIAYKGT | |
| | LDDLKTSICAAHAIGNRSRPAVVMKDIILHPLQLA | |
| | QAAQLHADGVVLNSFYLGPALESMIDTSYNLGIE | |
| | PIVEVHTLEDALYAIQLHTKILMINQWDRLTNKC | |
| | HPNRALQIREIVPDGIITIACGGIKTLEQIEQLGLAG | |
| | YDAVVLGKKLADTNIPSFVGSIKKWNAPGKGILAI | |
| | SKPLFFTDEMDEKDVSGGGHVIRMKQNYRQTLQ | |
| | EMCEFYQIAENNCVEAPRPKDVLLRFIGNDMPEK | |
| | WIFKREKWMKDHAHEYPSEDHATAVYDFNLAVE | |
| | LNTTLECNKKLLSQHFKREMVDKLEEAIKNYVKK | |
| | CYINLKRFTNKISCS | |

TABLE 7
B. microti secreted antigens identified in the hemolysate fraction: SEQ ID NOs: 55-
62

| Antigen ID | Annotated Sequence | SEQ ID NO: |
|---|---|---|
| BMR1_01g02545 | MSQGPAIGIDLGTTYSCVGVWKNETVEIIANDQG | 55 |
| | NRTTPSYVAFTDVERLVGDAAKNQDARNPENTV | |
| | FDAKRLIGRKINDPCIQSDIKHWPFTVAAGPNDKP | |
| | VIKVQFQGETKSFHPEEISSMVLTKMKEIAESYLG | |
| | KTISNAVITVPAYFNDSQRQATKDAGTIAGLNVM | |
| | RIINEPTAAAIAYGMDKKGTSEKNVLIFDLGGGTF | |
| | DVSILTIEDGIFEVKATQGDTHLGGEDFDNRLVNF | |
| | CVDDFKRKNGGKNISTNRRALRRLRTQCERAKRT | |
| | LSHSTQATIVVEAIFDGIDYSCNITRARFEELCAEM | |
| | FKNTLIPVEKALADADMDKKQIHEVVLVGGSTRI | |
| | PKIQQLIKDFFNGKEPCKSINPDEAVAYGAAVQA | |
| | AILTGEQSSKVQDLLLLDVTPLSLGLETAGGVMT | |
| | VLIPRNTTIPAKKEQEFTTNENNQTGVMIQVFEGE | |
| | RSMTCDNNLLGKFHLTGIPPAPRGVPQIKVTFDID | |
| | ANGILTVSAADKSTGKTEHVTITNDKGRLSQQDID | |
| | RMVAEAEKFREDDEKKKRCVESKNELENYCYSM | |
| | KNALEEEGVKSKLSSSELSEAQKLLQNTFSWIESN | |
| | QLAEKEEFEAKLKEVQAVCTPLTAKLYQAGGGV | |
| | PGGAAPGGFNAGGAAPSGPTVEEVD | |
| BMR1_02g03395 | MTAYIPGLLERLSAPIVSSRLDDDDLAYLGKYESE | 56 |
| | ISDSDNLQNIYSNDTIVALGDLDAKYKQQTYLSSL | |
| | NSHIVDQDAQIAFYPVKYTNKIIRNDSNLTYRRTT | |
| | SSLSNPLVNYIQKSQHDTPDPVDSNIQNSSSEQYAI | |
| | TTASSGSDKSKRLSHDNSKTDHDIHDKSGVVTAN | |
| | NHSTDRFQNAGDLVWQPGFTRQLGTKGRRVLLD | |
| | LIRKVYRGNPEYFKNILALKNPPTSISNLPFCNVT | |
| | MLWELANDFGVFDQAIQIHYAHGKPGYPANYHN | |
| | TANYGCNKTRKTNSGKSKKNKFYNYDYEAYDEY | |
| | YEDEYISSFSNGRTSRGRKIVQPKRFDDYDHLLDS | |
| | NYQVEYGYCSNRHLNKDGNTILYYKNKSHDYVN | |
| | LGELKGHKIERFTSQLIC | |
| BMR1_03g04775 | MKNDFNSVELPGHFLFTSESVNEGHPDKLCDQIS | 57 |
| | DSILDACLEQDPESKVACEVCTKRGMVMVFGEIS | |
| | TNAKVNYEEVVRNVVKNVGYDSEDKGIDYKTM | |
| | DVIINLDQQSHEIAQAVHLGKDADNTCAGDQGIM | |
| | FGYATDETPEYMPLSHSLATGLGKRLKDVRLSGL | |
| | LPYLGPDGKTQITVEYIKEGYGSIKPIRVHTVLISQ | |
| | QHSANVSNDKLREDLMTHVVKAVIPPHFLDDKT | |
| | KYYLNPSGHFVVGGPSSDAGLTGRKVIVDTYGG | |
| | WGAHGGGCFSGKDGTKVDRSAAYYARKVAKSL | |
| | VANGFCRRALVQVSYSIGIRSPLSLHVDSYNTCIE | |
| | GFTDLDLEQIAVRNFDFSVGNIIKELQLKKPIYSQT | |
| | GVYGHFGKDNPEYLWENVKDLSHELTHKPKR | |
| BMR1_03g03380 | MDYTRSLFTLSGPATASEVEKHIQNAIEFVKRRDP | 58 |
| | DQVQFIQAFTEVANGLAPVFQTDLKYLEIFLSLSE | |
| | PERVITFKVPWVNDAGKLMINRGFRVQFNSTLGP | |
| | YKGGLRFHPSVNLSILKFLGFEQIFKNSLTTLAMG | |
| | GGKGGSDFDPKGKSDNEVRSFCQSFMTELQRHIG | |
| | PDTDVPAGDIGVGEREIGFMYGQYKRLSNSSTGT | |
| | LTGKDPKWGGSFIRPQATGYGLVFFVQYILNDLH | |
| | NGDSFKGKRVAISGSGNVAQYAADKVIDFGGIPIT | |
| | FSDSSGYIYEPNGFTKEMVTVLMELKNIQRARVS | |
| | EFLKYSNTAKFFPNKKAWDVDTNVNVALPCACE | |
| | NELDKADAEMLVKKGCIIVGEGANMPTTPEAISV | |
| | FKAAKVTVCPGKAANAGGVAVSGLEMSQNSQRE | |
| | KWTSEKVLEKLQDIMKNMSKACQEAAAKYNVH | |
| | GDIISGANIAGFLKVAHSYCDQGCV | |
| BmR1_04g08050 | MSYSAEETDASIEQWKILRLIRNLESAKGNGTSMI | 59 |
| | SLIIKPKDEIARINKMLADEFGTASNIKSRVNRLSV | |
| | LSAITSTQQKLKLYRQTPPKGLVVYCGTILTEDGK | |
| | EKKVSLDFEPFKPINTSLYLCDNKFHVEALKELLE | |
| | SDEKFGFIIVDGNGVLYGTLQGNTKEVLHSFTVDL | |
| | PKKHGRGGQSALRFARLRLEKRHNYVRKVADIA | |
| | VQMFITNDRPNVSGLVLAGSADFKNDLMSSDMF | |
| | DPRLAAKVVKIVDVSYGGDHGFNQAIELSAQCLS | |
| | NVKFIQEKKIISRFFDELAHDTGRYVYGVHDTINA | |
| | LEMGAVEMLIVYEALDIQRLQMRNPVTGEESVIIQ | |
| | TSERDTEAMRDPVNNVDLELVESIHLSEWLVNNY | |
| | RNYGATLEFITNKSQEGSQFHRGFGGIGGILRYKL | |
| | DMSEYDLPVNDNDFDDFI | |
| BMR1_02g01430 | MDSLVPPYNKLNFDISRPDISGNTLHYTFFQYPDS | 60 |
| | SISKLRNVFAHNPQQNFTNQPFYPPKHNDTPHTEQ | |
| | NGSQFIHSNSNTSNNLESNDVDNNASASACDKRS | |
| | FPHDDRYSSSNYNEYPGILSSIQDIADLFDLDNYHI | |
| | YHGIDNISYLTYSTDANRTGIDSKLEFIYEVLNSNG | |
| | SNMTLSKLEGFLRILTTDDNPIGTLTPYGSLMAHT | |
| | CLRFLKSYIRTDNKGNSHDPDTFIYSIEGKKGKSK | |
| | LSKQGITNENETTTVSSDFRRIAMFNFELYSKQLM | |
| | DTIHSQDKSIAINKRKFDDLTSNYTKSESVSIDSMS | |
| | AISDSTDKRAVSSQTLKIIKQGSAYLQDFISKYRIG | |
| | FEPHFPRIVCGSIDRSLVTNQASITVPIDKSGRSVHI | |
| | AFEALSDSYGMSVWPELNQLPNSLQLGTKIQIAW | |
| | HKTPGNMRWYIGTIVQSTASQYSVQVYLGKNEY | |
| | KTRYCTMKYFVPPNVPVVKPDNTWWRLVPREIN | |
| | LSTDLYVGSCVSLYDIISNFESIDCVICNVYYSNDN | |
| | SPNGPIKGPNEEIYKSDKSSSGFKIQLNCACHGPTR | |
| | RIRQGVLTTGVVRVHIYCMSHKIDKIIPIDSIRGYK | |
| | LNYDLHAHFDGQPDYGPLMQSTTWTWVIPRLVI | |
| | APDNKYDDEKYWTSNPKYSLLIFPYHRNMEYIIS | |
| | ATKLIAILHPELNVTSLYNNGEWFVRQEGDIAIKG | |
| | YGGLRPSGLSFSKIPLLDRLFGCHRIKVFYNVPIDV | |
| | NKKVEDFKNDYTISRCANCRGIYYTDISHASNGK | |
| | TYISSDVEYRKKIQIAAAIRWKRVRESSNTILNCY | |
| | NENDELVYRTKNSFSLSWEQDGRNLELAYRRAH | |
| | APGISKFESKRLDSLMKSNKQVSNVPENMCTEGL | |
| | LEKFNESMKFSESVKSTDSKNAAKLLEVAILGGW | |
| | MNDSTKTESINTNKGDNIFRYINKSIQAKAAQDAL | |
| | DFLKNCSPPCLSKQVGKHYTPINGKQSMFKQGG | |
| | MSDYQDSIPHLDIGNPMISDYNSKKVKLQSVSGD | |
| | QRGLNIYENPCIATVHDPSVNYNSLRNFLAKAEW | |
| | DEEIEDAPSHGVLDFTIKSEEPVHNEAGEDCIYAV | |
| | PINDSVNIIWSDTEDAK | |
| BmR1_04g08520 | MVERLIARSLSIDSIETQILRFFSGDIGLQTVLLEFQ | 61 |
| | KGPRAFVICLDIINKHCKEFSSKSLPLILFCSQTLVE | |
| | CDHIYSLCRLVSDGQAQDLDKSDATKLDTEREETI | |
| | EKLERVINLLEQLNKVNPQIFPSLRFLLCAWLRLK | |
| | LFDNWSKGITFEIIQFVGTFQSNRQFQIELLAILAQ | |
| | EICNDKFILSIARRNELAKNCISQAPMVFKFLGVK | |
| | DSSIGSVYSDWIQLHVKYLDSIVDEEQCELPSDVD | |
| | LMVKLSLSTLISLNLIDKLFEQSPLPIHTITNLIVLC | |
| | PLDNHTALQDDCDMINPLVNEILTLIICNLKLYKI | |
| | QSPLFKTTSPILLSLSYEQLPYLIEHQFSEDIDNILD | |
| | GTIRILSNGDYPTRDVMVNFWINLKRNINTQNGW | |
| | EKLADYLQKIVIVFYEMPLYETEKYDFAELCSFRE | |
| | SASMLLFEIADAIGHQFIFDIVECRLQVLVETINLK | |
| | EQITVSFSEIESIFFILSAISSNAEMGKDTCIPTALAL | |
| | LNKLKYPTQGSLSLLLSISIGRLILWTAEYSGKKTD | |
| | LFNCLFMLIIGTLLPSILRIKNNSASKGMYYYLLGE | |
| | NILIDAMLALSRTSRKIVSEDTQEVKKYLICIYQIIK | |
| | NVQFSIENRIKAVNAAGAIISYMPIAEMKTLFSDLI | |
| | ENLSNNIKSVSNPTDYIQLYLMAMQSVCPIPDCIE | |
| | SEVAILKIVEKHVSVMELLFTSSNEDIIERLSQVLV | |
| | VVNRISRNHSEASPLFLWTLKLLSVSFRCSHPSHP | |
| | YSALRSILINVNDCSVENWASISNSLLPSLAMLKD | |
| | SIVSCCVGDKDRNLLQQQMSDLTMDFNTSQLLSA | |
| | PDSVGLCVDCLNVALNRHEIANFLLDKSKFIIFLES | |
| | LLIILPYIVHPKVLHACMILIKLVASIDMNEASGND | |
| | YSDSAVIGTSVYAKKIRLLNIISQRCNESSDEIHTIT | |
| | FKIVTAILSTVISGTCGVETWIDVAAETILALMCD | |
| | GKTADEAKNAIDSFFEMISEPSINVTIKNDYCNKL | |
| | KVPNTLFQALLQLEGWKLWKKC | |
| BmR1_03g00947, | MRGMFSNKWMSFVCFSILFVALKSDLEYVSALKL | 62 |
| BmIPA48 | LRAPPQTSLFLEKLIDDGSDIPKDPIDTDKEESQSS | |
| | LFKFNLNLFN | |
| | KKSIWEADEKFVITLAKSRLNVILAQKLDKFLAKT | |
| | CKIYTVDSEHSACINDIKIYAQKCIESNDLNSCYVI | |
| | PIQPIAKLP | |
| | TSRLYGLVPHVLNFSILIFTNLRSNLDRYYIDGSKD | |
| | WFSHIFMRLKRFFGIRNKHSYFSDNRLMNKIFSRT | |
| | STTFGPDRS | |
| | DSLLSNYIKFGAIEYAILLNTRSNLVKMILSSFAHI | |
| | KFVRKRLYKFYTNKWKSIEGLVTRGHLKPVDLSN | |
| | NPISDNIFKY | |
| | FGKFSNNTNLSNAIAGAFLDHYKSLFSNSTDVNGE | |
| | GSSGEGPSGEGFNGEGSSGEGPSGEGFNGEGFDG | |
| | EGPSGEGPSGE | |
| | GFNGEGFNGEGLNGEGPSGEGPSGEGLNEWNGL | |
| | MNGTA | |

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of detecting a Babesia infection in a subject, the method comprising:
detecting whether one or more secreted antigens selected from SEQ ID NOs: 1-62 are present in a biological sample collected from the subject;
wherein detecting presence of one or more of the antigens in the biological sample indicates that the subject has a Babesia infection.

2. The method of claim 1, wherein the Babesia infection comprises Babesia microti or Babesia duncani.

3. The method of claim 1, wherein the biological sample comprises one or more selected from the group consisting of: a blood sample, an erythrocyte sample, a leukocyte sample, a plasma sample, a urine sample, and a saliva sample.

4. The method of claim 1, wherein the one or more antigens further comprises BmGPI12.

5. The method of claim 1, wherein the subject is a human.

6. The method of claim 1, wherein the subject is a mammal known to carry a Babesia parasite.

7. The method of claim 1, wherein the one or more antigens are detected by one or more antibody-based techniques selected from the group consisting of: Western blot, immunofluorescent assay, IEM, ELISA, PCR amplification-based immunoassay and immunoprecipitation.

8. A diagnostic tool for identifying or diagnosing a babesiosis infection, the tool comprising:
an assay platform, and
an immunologic agent having specificity for one or more Babesia antigens selected from SEQ ID NOs: 1-62.

9. The diagnostic tool of claim 8, wherein the babesiosis infection comprises a Babesia microti infection or a Babesia duncani infection.

10. The diagnostic tool of claim 8, wherein the assay platform comprises one or more selected from the group consisting of: an enzyme-based assay, a radioimmunoassay, a PCR amplification-based immunoassay, a fluorogenic immunoassay, a chemiluminescence-based assay, and immunoblotting assay.

11. The diagnostic tool of claim 8, wherein the immunologic agent comprises one or more of antibodies or antibody fragments.

12. A method of treating, ameliorating, and/or preventing a Babesia infection in a subject in need thereof, the method comprising:
obtaining a first sample from a subject at a first time point;
assaying the sample using the diagnostic tool of claim 8 to detect the presence or absence of an infection relative to a comparator control,
administering one or more therapeutic agents to the subject;
obtaining a second sample from the subject at a second time point, wherein the second time point comprises one or more time points after the first time point; and
assaying the second sample obtained from the subject at the second time point using the diagnostic tool of claim 8 to detect the presence or absence of an infection relative to a comparator control.

13. The method of claim 12, wherein the sample comprises a blood sample.

14. The method of claim 12, wherein the infection comprises a Babesia microti infection or a Babesia duncani infection.

15. The method of claim 12, wherein the first time point is before administration of the therapeutic agent.

16. The method of claim 13, wherein the second time point is after administration of the therapeutic agent.

17. A method of treating, ameliorating, or preventing a Babesia infection in a subject, the method comprising:
detecting presence of one or more peptides selected from SEQ ID NOs: 1-62 in a biological sample collected from the subject; and
administering to the subject at least one anti-protozoan therapeutic agent.


SEQUENCE LISTING (62 sequences)

SEQ ID NO: 1 - protein, 215 aa, Babesia duncani
MKFLFGFFVILFLRLSKQEELVSLQLGDFHFDFENGKYSTHEPFETCFIDVYNYRYDKTGPFLFNIFIKRTLENEYQSLFFKRENGKLVNFAPSHLSSPQTDNTGTYYSTKEPVVLESKNLSDIRNGIKKIGGNKLSSSGKINWDTISTTLLLKTACGTYSTYSSGYEALLPVKDGNDTFCCCFSKAVLFTGYRFQMKKYEEVKPASNSQSTCKK

SEQ ID NO: 2 - protein, 644 aa, babesia duncani
MAATAIGIDLGTTYSCVAVYKDNNVEIIPNDQGNRTTPSYVAFTDTERLVGDAAKNQEARNPENTVFDVKRLIGRRFDDPTVQSDMKHWPFKVNAGAGCKPTIEVTFEGQKKTFHPEEISSMVLIKMKEIAEAYLGRPVTDAVITVPAYFNDSQRQATKDAGTIAGLNVMRIINEPTAAAIAYGLDKKGSTEKNILIFDLGGGTFDVSILTIEDGIFEVKATTGDTHLGGEDFDNVLVEHCVRDFMRMNGGKNLATNKRALRRLRTHCERAKRVLSSSTQATIELDSLFEGIDYNTTISRARFEEMCNEKFRSTLIPVEKALRDADMDKRKINEVVLVGGSTRIPKIQQLIKDFFNGKEPSRSINPDEAVAYGAAVQAAVLSGNQSEKIQELLLLDVAPLSLGLETAGGVMTVLIKRNTTIPTKKTQIFTTNEDRQEGVLIQVFEGERAMTKDNNLLGKFHLSGIAPAPRGVPQIEVTFDIDANGILNVTAMDKSTGKSEQVTITNDKGRLSQTDIDRMVAEAEKFKEEDERRKCCIESKHKLENYLYSMRSTLNEDAVKQKLSTEELQNGLNTVEEAIKWVENNQLANQDEFEDKLKEVEKACAPLTAKMYQAAGGAGAGGMPGNFGGAAAPPSGGPTVEEVD

SEQ ID NO: 3 - protein, 1058 aa, babesia duncani
MATIRKQRLEDLTDIHKEHLSTAHQLLDVIKSASDPKLIYLHCYQLMKLGGLDAEVPRLVVFGQQSMGKTTLLDFIMGGPIGYSSTDTGTRQPVVIIMRPESAIDPRELELASGSVGSPSSTITSKKIWCKFNGKIMDIHNVQQNMRLHMQSLGERICSEELEVEVYVPDAITAIFVDLPGIKDDSKSGAEFTRRVVRNYVSNNPNDLYLLVKKSSDDPANWPWSLREFITAAAPNGLGLSPQQTMVVGTRAREFLINEKTDIRTQDQLYERVHKRAVLDSKGQALPLHLLELFSLSIQAKDKGDFLTNKEEMKRQIANGQVEVENMIRHGFEESNSINKEGRSVSEELLDTFSIRQFLRSLNSKFSQLLNGHLTNLERRLIRKKIDLERIVASLETKLQCFSPTTVRESIKQFIRQFMEIVHNMMMGNYTIMKLPIPPEQFLGIYGGSLRDNLEDGHELAQNLFPQPDMYESNFYTKITARTEELYNKKLTMIDTVKPGRYVRYFTSKISVHMFGLIEPPRRVPPAVSGADLGPFDMMGQDYGADELINVEFIQLNNNEENSLHKDIDRSKLSLLTPLASVSAEHLQVPLYAWHKTKEPKTGWVVVRPIVIDRLPPEILVQKSNYREVDVANKQISFRYLDVEFETKALGSGEEDQLENSVETEISETPPVVEGVQNRCSNRYLYVTACSEIFLEEHRTAPYYASALEMVSGEHAEANLLNQLAVTNICHWLKFQIKHMEPEHVYSAEVLYQMLRSIDHVVDRADWEPLVADLVQSNVRGTLLHASRLAACASAAALRRVLRASLAEAFRCIKLTDCDQTLYCLPDSLHFQEQIDHLSEEYCRQKAIDCANAMMNCIIEQTYSIQFDVAVDIFDGCRQFEKYFMGRAGHRSFMGDALSSVKEDLALRKRRLAMTDIFEKSDAKTSIELIYEEVKVQFWATKLLLSTPLTTKLYTYFIKQVVDKALPTTHSDPTASIKVEFEQFLIDNILYEQIDGTRTAKSNNRLSADYDLNNKYEQLVQQHNRNKRLLEYISCALEGISRFKHHATADTDFLAHLD

SEQ ID NO: 4 - protein, 2541 aa, babesia duncani
MISSPPSGFSLHGEDAKGTPRDVERQDSDRLDPGNFPPDKYWSPSIGLSTIEANRRVVWLLQEAISRYKIHLGYWNTTKSCLTIDILIGVLVLVLFILGAEPNLGVWHVVRPVLLLPWLVLNCYSKITVMRIKSSRACERVRDVLESFSRELKTGGHTEVEQRRFSFDPPNSLLIPVYRDREWKRLPANVLLAGDVFKLQIGDYFPCNCRIILSCDREGKVQLDANLFNAGSVFKSSHLPSINTNDGGEWTDASFVAVTDSFVQSLETFLSADQGPQSRFMFFNERDWENCQKGPNTPPPTTCVWNFSDYTHFSKYGVDWIQLGIAFFISSIVTVLQIIWSGFQGWRRHVNVFATCIICLCHPAFDPFLNLADIWGNVKLQSLFQWHNEKRFTVDILPQQSSSSSSTFDSGSSEDSVDSEMAASRIPLLHQLRELNRVFKRGLDSEGSLLRTLCSVTLLCFVDDMGLLTEGCATPQELAVVDPSGGIGTQRRQQKESTAMFQSGNIDAIETINSTATSHKDSIGQTEQSDKGTIKAGPREGKQVVRKDPQGGERLVILDVFEDSSQYYKQYVKFNNDAERNCIPQVLSLSFAMSATQFPRVQPSLLQLKAAPDLIHTYMGMLANGTLHDFTHCLCAFAGSVGLKRSYIRRFRLLRFIVVLDETLGTNGKMLIYFLRDPRKQIVQMLVKAKPETVFDRSINYHDRARGAILPVSRLTKRKLRDLNMQWVSSGLTPFAFIYKPIHLDEFNLIMAHLPGVAVFKVGHFLKLDRHQSVKYQESTTCQQDPQADYFGEGGKSDAKARRWYARLTDYDAISMRYGSMHTDLRQRILTNSIGGLVNSCLKNSILLGMCATKYQYPKEVPSRIQSFHEAGIRFVYFSKHDEKQTRIVGGLLGLETSWNSMISLVKSGRYSHVNQDGRVVLPSGIDNIRRHIRDVDDIPLQVSLFCDCTHTSTVEMMRILRENGERIMCVGNGLRPSNFFVFCEAHSSVSVALGYHPTCRFCRGKRWSGIARAHAFEEATPEMKLSAFLTSLPCDLQTSKMYKVADPYFVMEMLHEVFKEARHMATNIQDAAAFFKLASHSVAWMLMFQASLGFRRILMPADLALLIFVYIPLMGSCLLSNVVAEGTMQQMPSRCTKDDAKVTIAAMCKTYPRLLLVATSLLVFYSFVLGQIQALLKIELERLYNFTLDDTQCSRFWRVASYSCLQEHEQSIALLRTQVQSVIFRENTAAHLAEQTASFAMAFLYSVSSASWIVRTGRIGAIASELLHSRWIISSAAILTIQGTILVARILAQPVPLASLNAIMPWGLVCGMLLGLSLAILAVDHLHKFYAARQHEMDQKNVESKVTSDLDIMDVTCICEPILSPSTQKISKRTSPPPIKQNVRGILQCKKRSYTSKSNRRRHPAIFNPDTTSAKDMGVLRTRQHYTSILLHFLQRGINGEQLQLVSLDIDQESFRLPLKKHSFGEEAAPAFLDVYVEEDIQKIPVMFNLVIETKGDYTYRNYFFKRDGDKFINFDVPKLSDYYNVRTEGIATYYLTYKPIRVPREVLKRLRREFPDNNQLKVSNDPEDGPRSNEITLKLKLNSGSLVSYMGTYVPKTSSRGCRSKAPIFNKFRYKLTTDGQAIPVDEVEEEGEEEEEENENEKTEETDDQDRKVVTKEDDEEEEDEEVEEVEEELVEIKDEEEDKCYIEAWEKHSKNYSKVEVATSNKKHISDTGSLKPIDELSGLFNALERKIATETALESDTTYTPKDNERWIIDKCNSEESILDLHIKEDILTFDVPITQLPIQEKEIVDPCNSQRKSAFFNKIGSAGEHYLRAFGSNILSVIDIMKSDLNVSIKKVNSDEINEDLPTKEKLLAMEPIMFNSMDGEDNNNKNTDEEVNGTKTLMHSINSQSIERCEMNSAYNQGSFISIHDGTSDSFMDSDSFSISQKITQDELLSKHDDKTLLNTDNALNDDSGLKTTEISHTVNDTVIPNLKLEKLLPQSKEQSAESTSSGEVGLIKKNKDVQSTKGKVGDLGNYVQINDDKEHPREIVTVEKFADETGIGTSTKKNEPATTSPLNEFDKIPNNDAAVNDSEKKTPADESRGNTNAKKTSDGNFKTISANLKPLKPFRPDSLVTAMHISNDNKFLATGNENGELYVWKFESAKMMSSPEISDNRINALPKMLNWEMLSSTPTAGVQAHEFFIYSIHISKIQNRKHSGSVLVVTSGGNNYVRIWALTKSDKENVLILKGDRQFDDDILTAFQLPISQHIAICGIDQVIEIWKFPPITTSVNDNPKNISSWNKTKERIDVELTIQSVSYSPSGMYIAIGSIDGLLSLYSAETMKLISMAICRNEKGWYSNSASITGIVWNTKETLVCATTADSRIRLFSTNIENDNALIYAEKLKGHKYCGENVAARFTGLNDEYVICISKGGYIVIWKHSCTEERIFYDGNPIIKNTNYCKFKIIPKPYIGKLLGVFNPGTWGHLWPTYQEQSVYEPLNDKNSVGCLDRFLTKNTPGVKFSKANPKNAILLVASVDGSQLLCTIIDASLLVFKKS

SEQ ID NO: 5 - protein, 2541 aa, babesia duncani; identical to SEQ ID NO: 4

SEQ ID NO: 6 - protein, 160 aa, babesia duncani
MPSQGTVKAQTGTTEVDNADSSEEESQKSQPESKAVNGISGTESRNENSQSVDTNSSGDTNPSQSAGGSAPTTGDSKNQQGKNVNAESSSNPNSEKSVATQDSSTDQTGKTDSSTTHTTTGDSVEQKGDDNTETTDTAQNTATEEATTGSGTEGSADQTE

SEQ ID NO: 7 - protein, 132 aa, babesia duncani
MARFFSYKKLIAFAIVALASLKEVSFLGGCPYALAVATTTTTGTNGAATGTNGAATGTNGAGANDTSKNTSDPNTPATPPSSPESNKDNAAGGSDGQKPTGQDPQKPNAGNGFAATSVIGAATIGLLTLAFN

SEQ ID NO: 8 - protein, 188 aa, babesia duncani
MTKYSQEPSNLAKSAKAYGAHLRVHFKNTYETGRAIQGKMILEAKRYLNDVIEHKRCVPFRKFNGGVGRCAQAKAFKHTQGRWPEKSCRILLDLLTNLESNAEAKGLDVENMVIENVLVNRAPLGRRRSYRAHGRIIPFLSHPCHVALIAVEKDENVPRFTPEAAKTIKLNKRQIARMRLCNGKGVAK

SEQ ID NO: 9 - protein, 490 aa, babesia duncani
MISHYCLLLLAAATVFGGEATVTIEDAKSATLEATTGVPEVAEGDVSIPRGVVTLTADDLHKSIEKHEAIMIKFYATWCGHCKILAPEYIKAAKILEEENVNVVLAEIDAVAHSDAVAEFEIKGYPTIKFFKRGIPIDYNSDRKAETIASWCKEMLNPALMETTNLEAEIASRKSKIALVAHGCNDKDELCVLFEKLAEVHRMDAHFFSVADSSSVWFEVRHVGDGTLKFNGLSPEELALFVKDETLPLLDEINPANYARYTSSGKSISWLCANTQDYTKYRSSIVEVAKEMRSHTVFVWLDTEKFSAVNEAFAISKLPAIAHQTMKGRFILSPDAYDFTSKSAMLQFYTDVEQGKIPLSFRSEAEPQDATEGPVMLVVGKTLQQLFTQTDKAVLLMIHAPYCEHCRNFMPVFEDFAKTIDAQAPLIVAKLDGDANESPLDYVSWEAFPTVLLFKAGDKQPIPFKGTRTIEELTSFVQEHVTLAPVKTEL

SEQ ID NO: 10 - protein, 350 aa, babesia duncani
MVEELDGTKNEYGFCKSKLGANAILVVSMAAARAAAAHLNIPLYVHLANLAGKPTNKFILPVPCLNVINGGSHAGNMLAMQEFMILPIGAGSFREAIQMGSEVYHTLKKVISSKYGQDATNIGDEGGFAPNIKNAEEALDLLLEAFRIAGVEGLFKIAMDVAASEFYDKNTGMYNLGFKGKEPQNKTGEEMISYYKLLCAKYPICSIEDPFDQDDFDSYTKLTAAIGEKVQIVGDDLLVTNPKRIEMALGKKACNALLLKVNQIGSVTESIDACKMAHANKWGVMVSHRSGETEDTFIADLVVALGTGQIKTGAPCRSERNAKYNQLLRIEEELGNKCEYAGHNFRTCGN

SEQ ID NO: 11 - protein, 126 aa, babesia duncani
MEEWYTYALRHVDLDLDNFFVEFGETILEDYTRIYPRSNVFVDNIRKGATLITLSTEHKGLLFVELRYPRPGKDSVMDIIYKNWEDEEVMFSLVWVFGDWVPQNFLYSGILDSGPEYVPVRRHDSR

SEQ ID NO: 12 - protein, 131 aa, babesia duncani
MQIFVKTLTGKTITLEVEPSDTIENVKAKIQDKEGIPPDQQRLIFAGKQLEDGRTLSDYNIQKESTLHLVLRLRGGVIEPSLVILAQKYNCEKMVCRRCYARLPLRATNCRKKRCGRCSQLRPKKKIKGGN

SEQ ID NO: 13 - protein, 218 aa, babesia duncani
MGNPGLIFIALFKYSFNCYYTFLPFKFDNNHFDVLPHENFLPRATVPRFVDIYVSADDPRIPILVNFVTGGMTTSGPDTRPTNRNYFFKRDGNKLVNYKFTNKPNDTVENNDIVTRISRERVTYYVGTSNPEDIIVATKDPQNDLNQRENSMYASLATLDPKATIPRIGLKTKSKYNKVALYSDEHKEKIEISNADIDGVMHEKLILFRYRLVKEYRV

SEQ ID NO: 14 - protein, 797 aa, babesia duncani
MRNVKKRTVPFLWISCFVLYGIYNVEPIALYNKNWVHKRVGFIANLKQPPCGITFQGLTHEKAKRGFKRYAEQSSQVKEASVLFDTLSNTEVLDELDSNDAIQDTPDPLESETIIEVEVEDKGKKTFSDIHNKLKGSINKLPSTFAVMKDLVAFLDTMEQQGNEKAKSIAYTSIKKKLITELNKNVEELKSDLPKTKKRLYISPFLKNTVDPFFEDQYVNPVDFKNYFSLYDNYKQEYRKIAYLCMKTYIARLILGLKRKLKHGIVSTFLWPFAKWAKDNNVRYKEPKPSQLENFERLLKYYGHEFENVYFEQNIAMVRPPQPKARPPNKWSLIFLGTGSRQPTDTRMTSTMAFTEHDGGRIWLFDCGEGTCACMQKLNLNPKAVDRIFITHLHGDHCFGLFSFISNSARALPITVYGPIGISKMLIDIMNFTTTSVLPKFVVHELVLHPDNEKHKTGWHVNYPSFGGYIYPQEAGHYLVYENDTCKVMAAPLKHILPTVGYVIKEKSKNENDSTKTQRKIVICQDSCDSSKMVPISMNPNVLIHEATTSTTSTIGSSLIMQLVYNFSKGKIDESLLSKINDIISQEELRRSCFSVTFTTMAMKLRRKCYLIERSYSNLTKTLEQNELKATETESNNADMMTDIYNIVQLVHAASKIKSCIAEAKKIESQLKELYDAPKHLGPRSTWLKSVYNHVRDVIENNGNTETSSSPTIQAMESLKNLLTKFWTTNKSMGLLFSLEINLPEAAPKDWFSLYSKSVRYSGHSTPWDAGKFAAKINAASLYLTHLSSVSLHVHFY

SEQ ID NO: 15 - protein, 380 aa, babesia duncani
MLLSCWFNLIACFICICSCYIRLNQTNCTNLRLINTNNSNIKMKATGKLSMSHFKNKQGLLIRTYAAEVDKPKGSAILVHGNKSHFRADFTNYNVDFYKDKYGLESVDPNIVIREMHAIYPNVDHKIDFNNDYEFHYSKLDGKNALDITPRFTLNGSIVEYLNGLGYSAYGLDLQSQGMSQGHNGHRNYFKKFDDHVVDVIQFIDIIRRNKFHNVNEEWDPNVLGKNYSLNKCFLMGLSMGGNVVLRAAQIFKTLSDFKSNIVDGIVCFAPMLDIDMHFSGAFNQLALAIAKMIVTFCHHSTFMINEKYEIDTLNSFLRVNDPYYITKTQTHKAIVSLLEATRTLEKNHSKYPVDMPTLVFHCKDDNVCDFKVFTCNEMI

SEQ ID NO: 16 - protein, 713 aa, babesia duncani
MADGMNQETYAFNADISQLLSLIINAFYSNKEIFLRELISNASDALEKIRYEAIKDPSITEDQPEYFIKLYADKNNNTLTIEDSGIGMTKADLINNLGTIAKSGTKAFMEAIQAGTDMSMIGQFGVGFYSAYLVADKVTVVSKNNNDEQYIWESNASGHFTITKDESGEQLKRGTRIILSLKDDQTEYLEERRLKELVKKHSEFIGFPIQLSVEKTTETEVTDDEAEESADAEGEKDKIQDVTDKDETEAKEGEEGDKDKKKKKRKVQNVTREWEMLNKQKPIWMRSPNEVTNEEYASFYKNLSNDWEDHLAVKHFSVEGQLEFRALLFVPKRAPFDMFESRKKKNNIKLYVRRVFIMDDCEELIPEWLGFIKGVVDSEDLPLNISREVLQQNKILKVIRKNLVKKCLELFNELTEKKDDFKKFYEQFSKNLKLGIHEDNANRTKISELLRFETTKSGDEAISLRDYVDRMKPEQKYIYYITGESKQSVANSPFLETLRQRGMEVIYMTDPIDEYAVQQIKEFEGKKLKCCTKENLELDDDEEANKNFEKLKEEMEPLCKLIKEILHDKVEKVTCGRRFTESPCALVTSEFGWSANMERIMKAQALRDPSITSYMVSKKTMELNPRHAIVRELRQRAESDKTDKTLKDLVWLLYDTALLTSGFNLDAPAEFGNRIYKMIKLGLSLDDDVAEASLDEVPALDEVPVDSRMEEVD

SEQ ID NO: 17 - protein, 193 aa, Babesia duncani
MRTLSRLPPPGDPGHVASFQEIATFNIDEFLANIDKTHDGVSQLQLPPNLLENLKQGAGSLLQHLAEQSTGPFATHNQPSLIGASGMAVAAPQMQLPQVAMAPQVMAAPQPGMPPAVAMPQMTSQFAGAPGQALPVAAQAVPNMQAPAGPQLASVPISNVIRGNNIAFPGAVPNEELAMELVMKITWALLMHC

SEQ ID NO: 18 - protein, 255 aa, babesia duncani
MSEENSQRAQLTYSAKLAEQAERYDEMADAMKLLVETCITDKDELTVEERNLLSVAYKNAVGSRRASWRIVSSVEQKEASKSNSVHKTLAGEYRAKIEKELNKICLCIIGLLDEKLIPATVDSESHVFYYKMKGDYYRYISEFSCDESKANASASARDSYQKATEIAESELKSTHPIRLGLALNYSVFFYEILNRPQQACEMAKRAFDDAITEFDSVSEDSYKDSTLIMQLLRDNLTLWCSDVTSDAPDKQKQED

SEQ ID NO: 19 - protein, 742 aa, babesia duncani
MFGLLRPCLIRLSGAVATRQTFGNLADCLKIAESSQSIATTDLYEAFKFISSSGQIRRQAIHDDRFVTLLDQLDARISTLNCSYMGNFGIRLGLIIQSLGNLDREDPIVEKSVKVIERLCTEMMEKSGNIKEISQLAFAAASAGLQHKFLDYAKQNLTLNIENADPDVLNLALLASYKTKVHDKVFLALICEKLSELTDRFTANDVVSTLRSLEKTSLMKGFLLRRLSMLIHDNLEQFTNEQLAQCCYRLSILKFQTPVQYSTILSLLEPKFQQLSIHLQIEVLASGCMCQCTDANERLVKLAKSITLTDKVDLAGLVNYIYSCVYLKLYKGDHLTGALEEALARSPFLIRKYALLFKEAYDTLSLECPNLTLELPEAWKMALENYESAEHDRCIQTSIIAETGNILKTSAGDFETFSKVGPFTVAFADVARKLVILAETPNTLGGLALAQRSIKAMDYKVAIIKYWEWRRLKTEKSELSYAFKRFDKSRLGVLSHVQFVRLLGAIGIHLTRQELKFLQFEEDIRGGFTLEDLEALGRDFYNDEVIATKVLESLQEHFGPCNTLDKQELASVLMKLGASLGVAREELDTFLNFYSAHSNSISVEAFIRGAALGVLELCADHTVTILQWETSRLQTKTSKMSGRGKGGKGLGKGGAKRHRKVLRDNIQGITKPAIRRLARRGGVKRISGLIYEEVRGVLKVFLENVIRDAVTYTEHARRKTVTAMDIVYSLKRQGRTLYGFGG

SEQ ID NO: 20 - protein, 400 aa, Babesia duncani
MVLSPILQFSFLALPSMLLNNVFALRMSAPTPIESPVTKYGDSLNMLFLDLGNAFHENFSMFQVATSMSNYAETNNDIVSRICERFESQKACFVLASKYINNCAKAKKCMQIEKFHLFQSPDMSIKLMNRAQLAAAIHVFRNSGVYEKNYLKRRFNKIFKRQPFGYSSYRTLLIPLLYSNASFNEYTTFSEIFITYYLNVATFMYATLIYRDTRLARFVNAFDKLNVLLIPLKKHLRNMVTGIANASPVAFCEEDFEPILRIFGHYLSGFDKSLTPLANRFAKLIRDVLKNELHKSESCILNRAGDFLKNAGRRAGKAVRDAGATIKERGMATYKGVKGGLAGASDKIRNRFRSRNGNGDDASSYGLLDEDATGEATDDVKPEDDSTGDNEPKEDEITRL

SEQ ID NO: 21 - protein, 242 aa, babesia duncani
MNLKWLLGLALIGSKYALGGDPNDSEVDSGKERGPGKRMTFDELLDELKTAEASVLGIKAEINGGLNRLRYRIGNLDAITKSDYDEISDAIRDIITKRTEFAKAVNKRVQLEAIANKFSERTSMGNLEDIQFSTFWVKLEAITRVPDFQLKEDFVKMKDEIIDVKEKFIEKLKKAREATAEVIPETIVEDQEMKSDLHEEIKSHGDDDIFNDKSDKKQNSGFAATSSSLILLAMATIGYSLF

SEQ ID NO: 22 - protein, 917 aa, babesia duncani
MAKSALYILDSNVKSKKVDKESLKLIKKQSGKPKKILRPVEHKAHNPSCLENAKVELDLDLLKKVIETLKKRAEVVRESNTRDLLEDPSRNYVFIQIALTKVVTEVHVKPLQIKLKHPIYTDKEVCIFVKDPQKHWKEIIKRENVPQIKKVIGVTKLKKKYKQFEDRRKLCRSFDLFLCDKAVCCSLPSLLGKVFIQRKKMPVPISMSKGGLGNSMREAIQSTYYKLSTGNTCSVKVGICSMNTEQLIDNIKQVFQTIKKFHTEDPIFRNVISSIFLNWEGTESLMLYSRALADDDIQIPQSHVTSPSKPTAAKPTCKWFGTSQSDLVARIVATAKGGTTSLSVWRQFSKDVIETIDTLNIPDVYRILKCFSILRYRHDPLLNVISYRIVESLDKIACKNLAEILKAYSKLECRNDFLLKTALPTVARHLEFFTPSDLSSVFYSYCNLGFHDLNFIRQVEWRIFNTLGKLQSCDFALLFCGLTRLERINTRFVISLACQFCKSLDAIDEKHFSLCVNALGRLEFAEHPHLYGILVQHIYNEIKLHHLTSVSLALLVNGFSRAKPKDLKVFQMLSKQIESRLSEFDIHSLCLITAGYSRISNAMEQVYLFEKIAESVGRKSLQLYPMAITSLMYSFSRAGHVHGPLMFYGSQHLTKFAEHYNIVELSMVSRAHCLLEIKNDDLMHCIAREIVKRFPNVIPTAADSPRVRRISQDSKDLEHVPQESEKCLILEAGTINLLWIMQGFASFYIFDGNIRNAIMAICNEMCMRIVDLTPMLVSNFLHALATLRYRHETFLEILVRELEDPRLGVKFNQDELKLCYEALNTFGVHGPIYKVSKMALKQAHEINGGDSTTTLRQVMAGELKTFQDDSEELTQELKIPPPPKKKVYIHVPEVIRKHLQVPNAQVTHQYDFVTFQV

SEQ ID NO: 23 - protein, 618 aa, babesia duncani
MDVFSILLVFSAFYVNAIAADDVKTFLFKKDVESTVEIDANDDAVLVCPIASVLIIKKARWLPVTGGDMRVKDGFSRTTRIGWLCNGLENCAFRPVAHLSKIGDRYEFLGQPIETDIYKLTVTATCGNFMFKRPGRREMLCIPTSAKPDIVLGCKDNEAIELSYVRVGGKSKHQWRHRDYCAESIIKTAHPLCTGKKTCKIAHDVFLKNAKECIPREFNVEYYCAAPHKNSFYDPLDAVVVDGVSVATKYVLTAEDGARASAKTNAYQVLQVDSALWESDGATERRDRLELVKFLCDGRAECVFSPTRSIIGPDERKCNDVVFGGMVKDTMSHFMLRAHFSLVPFDPKKYDEKEYHHVTIKSTEKKTLECPVNMSLTFYVALWGGKITDTSPLKGPKHFVEVDINGEKHRYSEIINIVGTQCFGKSKCEIEPLKLKPPRHEKDLKEFPTHEGVKKDDHQLELYYKCIDLQTLPSLVESLISDGPRYPREFITPIQLSPDMRIVVMLDIYGPTVLEVANALKLEIPVARTNEIKISWKDAKISQGIRLVKDTRNYVFEFVIGAEDYIHMTVNSFDNDGSPMSIPVEFEASKRILDFSRGIEDFVVATGEITNFRAFIKS

SEQ ID NO: 24 - protein, 274 aa, babesia duncani
MVTREMYIMGYANVISADIKKFKETLANEITTREEYEKLKADIASTRAIMGDLKSNVRVLTRLLNEVYILKTLMREEDVRALGDLSMEAKTLVAGKKELQIKLENEIREIKKKVDGLPLTKKILEENTEQLLEEISKIEMHVKKSAEAIEKNIDECNDKITQTNVMAEEEHEALTNKLGITKIVLNGIKTGLLKLISISNRLKAATSGATREENKILKETILNRIKFILAEGDVVFKRLEKDMTDIEKRIKRIPIEGTLPDLNTHGGYGTIETH

SEQ ID NO: 25 - protein, 498 aa, babesia duncani
MDFLWLLGFAIIYRKFVVGVGPDEDSDYPEVDVKSSKVNIGISATVDKFFDDMKLMEEDYKAYQDKILGALNTVRRRLEKAKHLEATDLSDLANIMLDVNKELTKMNSCVSRLAALRPYVEKSINLLHEDDKETAKKRLLNFMNPDEMVNVLHMLFSEYKELNVELVHVKKRGTAPSQSESLSKSKIESLSKSLSALNQEPGRILEPALAIYDKIMNGGTEILEEMNKLNLKIQGKQTMSSMEYLYIVQNMLHAKEYVMESKQPLTSLGYLGTALDQMQFTYSPTEKSHVENIKKEVGEILNGIKDFQNKVKNSIDTVEKRVTTISVTDALPKERALEIISAVPAYVQIFKKQMDELKGAVLNDINELDKQLDEKKRIPSKEHEQMEAKIPILETQVQFFLEFIEAMKYFRVTCEMIPKMMGVDEKKQFRRELILCDMALKNEKQEYDFMIEQFKKVKERIVQTRPRASRFKRKHSGFSTMEPSSLLLVLPVIVSLFY

SEQ ID NO: 26 - protein, 414 aa, babesia duncani
MGNACCKSSPPAAVSDAKTADKPNFESLSTLSMKNDGSHRNQEKAPSAKASPRKNVEFKFTDTGYDATGAKKWDEKVISALLGGKPTNIEAAPGIDVGDGLVERGPVLLKDGSVYCGQWKGSVRHGRGQFFDVDGTQYIGNFSKGVFEGAGELRTWTGDKYQGLFKNGKYHGKGIFTQKNGDVYEGVFVDGMREGYGTERYKDGSVYMGEFKGGKRMGNGELKMADGVLYEGEFNDEITGKGKMFWPTGECYVGSFLKGMKHGLGETTWKTGPMKSQRGKYENGKMCGTFENVMRDGKVVKGVYKDGILLQEITDTKPKVAPVVTQPPPVKEQATPTPTPKAAASPTTSTPTRAAASASPTLSRAPSTSSTPATATPPASTPTAAASTQAKPKAKSAKSSSAAKKKTSSKSSAR

SEQ ID NO: 27 - protein, 376 aa, babesia duncani
MADEEVTALVIDNGSGNVKAGVAGDDAPRCVFPSIVGRPKNPALMVGMDEKDTYVGDEAQSKRGILTLKYPIEHGIVTNWEDMEKIWHHTFYNELRMAPEEHPVLLTEAPMNPKANREKMTTIMFETHNVPAMYVAIQAVLSLYSSGRTTGIVLDSGDGVTHTVPIYEGYALPHAMMRLDLAGRDLTDFMQKILAERGFSFTTTAEREIVRDIKEKLCYVALDFEEEMTNAESSSEIEKSYELPDGNIITVGNERFRCPEVLFQPSFIGMEAAGIHTTTFKSITKCDVDIRKDLYANVVLSGGTTMYEGIGQRMTKELNALVPSTMKIKVVAPPERKYSVWIGGSILSSLSTFQQMWITKEEFDESGPNIVHRKCF

SEQ ID NO: 28 - protein, 292 aa, babesia duncani
MAIWKLFVFGICGAGKVLGYRLDDVLQGDGEDFALFGLGKEVIEARMDKLFSVIDLNNDGILDLEELAAFHAKTFQTILDLQLNHEMELVDRNKDGFVDVEELKVAFEREGTQDVDISTVEKGLQRRFVAADKDQDGKLNRQELGLLLNPGRDEELINIEIEEIMQTYDQNGDGLVSLEEYSHGRSDQEGVSAEFKPFDSNADGFLSREEIRGVYVEENKNDLDSEHEDLFAITGKKPITREVWNANLNKIAHTSLTDHGEMLRFPEDYHMDLGDIPRDRKDGAEERPSGEL

SEQ ID NO: 29 - protein, 476 aa, babesia duncani
MCGNGIINNLVYLKMDSIRESTSFHNDFYYITCPFSIKGFLKEYWLSTITFFLTFAAVFIPSVFKLDLKDHADYVMLPANMFLRHIRGFVVLFMFFASAAKIRLFLQRKVDSLKTRIMIKYLIAGLLSLLVTLGLAALIIPLNTSLGGNTYFSPHSIKDYNPTTEFKNFINHLAVHDLPLNMATTRVDFVNEFGKDQLHEGLGDGYNAPGFVVYGLLFAFAIYTMDEHTDALCNIITAMHKCLLSVYWILVVYSPFAFFLAGLVTFDELKVKGGLGIALMGYLYLTLAVLAVFVAWSFIVVPLLHFIRTARNPYPTIIKLLPYLPTAFCSGSSVLSGEKTKDFLKKRGFDPDDVENYLTFSTLVNFSGTTSGFTVCAILMLKLFGKSLDWVTVLKIIVTGLLVGFTIVEYIQGYLFGIIFFLNNTMLPPGSIILLLQIDWLLDRFRIVSNVIDDALTIDEITNVKSKLSSCPFHKV

SEQ ID NO: 30 - protein, 967 aa, babesia duncani
MGEGTNKTQYPFILGLHEEVVCAAKRLEICLLEKIEQLGHVDDKGWKVCVDSTIEGKLLGNHITCLSLCKVPNGSHGSEFLLAVGYKSGLIALARVDSQNEFHILSHNDQNKSSISQIELKYVGESTAAYNLMSLRVYAFLDSGKIITFSWGDKIQTSIQSGEHVLGFAVNSQNTTIAILQNTGIVCRSLDTETILRTLENVQIVNANRHYLSWHPYKNLLVFLDNKGISYTCHPKWDVYKFSQNSQHQELIRHVQFGICGDFVLLLTASFDKIIIWDFETESILYSHKGCDMVACGLIHLSNKRVRLAVFANFASNEFWRCKRLIMSGDELMEEAKSELPQSHKTRRLKRHITELDDYHIRKMVDQEAVDEDNEEQDEEYKDEETYRDILDSYDNLDKDEMTHFDQSRVHVMHEISKLRKKVASLDNRVAERTTLVPGSCPAPDDAAVQWVLFWDEVGQITKQLISDVWCLHVHVFSGPLAGYKRKPDRYNCHTAALNQKYVVTGSEINVDGGHVHGILTFLDFENNTQWDRRFFNEYISAVAVGDSFVAAITADGILYILSLARSLMGVFQLKGSPIAIAARGNVLATITEATTLTNVSSFGHVRMFWVNGLRGLAKNPASRIVDLYSDSLVLGPDKAISWISFSSQCTLWITDTSGQLMALLPAIESCYKVGGYSLEWIPFMNLENLVNESSDYEPSHTVFPLYVSDMKLNYILLKRGQSYPTCSQPLNFMGYTLKKACLRIDGCVGAYLPFQKFNGLVTKDPQLREVIGSDIISDVASIPWQQYDEMRHIQSLQACQNEYILKVFQNYNFWMQNSQGISDDAISAFGNAERVHDKWTLRILRKTKDSKQDSGVLLDALWMLRFPKCLEAAFSILQNQLDAKQRQVLQEASLLLENVTPFENNVQPLDAQSQPTATVSIPKQDLDPIPKKHAPLIQGPSNRLQEFVPLGNEPEESGPLFKNILE

SEQ ID NO: 31 - protein, 401 aa, Babesia duncani
MVDFEPESCRKHVDKNTRIYVDGAFDLLHWGHLNALRQSYKLGGELIVGINGDVETFHAKGISPIYNQDERAELVKGCRWVNEVMVGTPYEVNLDFLVNIAKCDYIAHGDDIAIGASGKDAYDEPKKAGKFIFFRRSLGVSTSTTVGRLIDALESDHFSHLSKNSENKLQYGDFEKALQENEEQMINEGIIDDALFSGNNKHDNKNKKSTDVFPYPRFRLSTSLLGEFITPKPKPKGGKIIYVDGSFDVFHVGHLRLLKRAREMGDYLIVGIYDDQTVRTLKGTPFPFSSLMNRALTILGMRYTDDVVLGAPYVPSRTYLENLGITTVVTGKQHDSKMINRDFDPYREARDMDILVEIDSGSDITTSDIIARVSSRMDQITANIRKRCAIEKTRKNIVCSL

SEQ ID NO: 32 - protein, 516 aa, babesia duncani
MLNNANTVKEVGFRKSVIYQPNVFKDADGVIKIKTNNKNIWIRQNSKCWEFTSKTPITSVDDYYKELLSDFNTDDSTSITLYNANVDLAKRGFNNFHEGYTKIKKLYCSILEAGLMIPYSGMLSGDFMYAVVSLPDKLDITYCPIDNYMENPTKSECENINRWMEFFKMYGTHVSTHIITGGKIFHEYKTLNITEYRKKSKFRQSTNIFEVTNINFDSAGQKSTKNTIVFGGNYVKGMESEARHFYNEWSKTLESRSLPIKVTVKPLSIFMSYRNDIYKEALKFYRDVALLTTVGIQYSTKIDELLRESTTVVSDDGMAHCPTNQIVLAGFIMSKNPKVPIVNCEQGKILCSNGTDKPASVYIICVKELNDIITTSTSMENVHICPNGNVTALGFAFRKQSESDDWTVVIPRIGKQQMINYTKGLNMSWLLCVPIEIMFWHMEMIIDSAPKDDSRTVSCKTGWTILKGFKLMFLKKEDTTRVVLEECISHQQHCILNCNEECTKMYGTILCKKQHD

SEQ ID NO: 33 - protein, 2356 aa, babesia duncani
MSTNGTVRWRTSSRSQLRDRRKNALTRRLRFEVGRLDTEAFETRTKYLREIFLDVDKRNSHDWTALEKKILLLDWCQNPQKFEKWKPAATEDMQSRFFANLIFNYSHLAMINSGMCIVIVRLVPLVVNIKAPYQCVILLVRDNVNGMVIDRLKVGLIWRGKDTNTDVLGKSTRIFTPPLSTDFQFLSHGIGYNFKSLLPQTEFEPHVACSDCISRELESLCTLETPDIVLESPALGMEVTSSFVNGHKVYLVVTRGVDGIVRIHRVQELIQTSIDIIRTWEQLRLKHLKRTMQSQQYAACTGCLVHKEELQQLYHLKQCARSFAPECGPMQFVGLEMIDVARPRTCATKREACGLPLALGYTSVRNEQAVIPSLLMAMASNTVCIWSIYSYLKEWNLNPLGPIKVMTYPDGTLPTDISSTVSIAASLEALERLEFIHAPKFYTTDDRGHLSLWSLGSTGPEAQVTLDQNALCSCAVNRSYPHIVAVGLDVGKIKIYNTLTQGTTCIAQMEHPLLTKVQTLQSFLPDNVYDYQRWYHPVVKLEWISDVFILAQYSEPIFTTESTNASAVAIWNVVKDIFDRDDALVCNKHWSLNSCNLYNTWHLASKLVCLYGGHFGCICGVLSSDAKWSAEQGLLAVTIDSTGQLHVFKPGIWTWGDCDDPMAIARLTGDCEFYHSILARVQEQHRRLSVPVMEIDNSVDMGQGTRLRKTRAKFSTYIQDAQTQLESVETASGLEEINSLEKLPMFCKKTLRLNSESEKYLKLVHRLVAEREHAEFLLSQGTPGKTGKRKCPLGAPNIKQEPLELIEAPQGPSSEAPVKSKNEYQNDAMEPESFNFSSVLTKFDTDVSMEPETELVETALPVVQPTKKRLVEKGLEACCIEGVQFDIMGDVEIARCAELEVQKRELYYYLSSMPFPQGVLDLRLGCNRNDTKCETCGRALMECVGHWGYINLQLPVFHVGFFKYTIQLLYCICKRCSSLLLPFDTVAQLRDARLRRSDDPLARAVIFKRILSSCRKVTKCPACGARQGVIRRIVKPTMDQFMKLRHVVKYKEGGKIVIVEDELNPLSVLRLFEAIDPVHARILNIMDPQRLIISNLPVPPACIRPSVSLQGQGTTEDDLTCILSDIVELNNVMATQMQQGFQTNQVIGNWQFLQLQCTRLINADAPAVSQLLAAKHISKPGRGICQRLKGKEGRFRGNLSGKRVDFSARTVISPDPNIGIDEIVIPEYIARRLTFPEKVTSANLGVLQKAVLNGVSKWPGACYVMKRDGVKCTLRFANPKQVAESLQIGDIVERHLWNGDVVLFNRQPSLHRMSIMAHKARVMPGSTFRFNECVCNPYNADFDGDEMNLHMPQTYEARAEALHLMGVLQNITTPRNGDPLIAATQDFLSASYLLTSKDRFLSHQEFCQLLCYVGDGILHAQVPAPAIVYPTFLWTGKQVYTAILRQIGAVVNLECREREFQHPEPNLFGRMQPNFMCIKDGHVIISNSELLCGALAKKTLGASKDGLFYQLLRRHGPHKAADVMLKVSKLTSRWVSDFGMTIGLDDTTPSPMLLATKQQLLSDGYAKVELAIANAANIEPFPGCTRKETLELQVKGILDDLRNQAGKACNKSLSANNKPMIMFNSGAKGALINIAQMIACVGQQNVMGQRIHHGFIGRTLPHFEVGCIDAKSRGFVSNSFFSGLDPAEFWFHTMSGREGLIDTAVKTSETGYMQRRLMKALEDLAICYDYTVRTSDGQIVQFIYGDDGLGASGAHATTPKALQETLAHVLTLSRFQRTPTILNSRAPGPNSKGDLRLPLPSGDGDDENFECGIVPKCKLAMPPKVKAKASARASWRTSQVGTPLDEATSAQINRNMYAHWVVHVHLDPEHAKAPLFGDEVLDWVALLEPLCREPLPRCMQESVTSMDHTSEPRIHASSMEIDKRVNGCSMKATNTGPRFSAVLLDTIRQWAARLQTLDPEIQREYESSGMDLVQFRAFKIPPQDRISVLQIYEFLRCAWSDYLHGICEPGEAIGALGAQSIGEPGTQMTLKTFHFAGVASMNVTLGVPRIKEIINAASVIQTPIIEVPLAKKDSYDFAKSVRAKIERHTLEQVVHSIKQVYTPGATLLLVSLDSQYIQQNMLQLDASSVCAVIRNSNLITKFKLSKHNIEAPQKWLVSIRLVASEALLFQLNALVAALGQLVVCGVKDIKRCVVKREQKDVINSLTPGATFEYALAVEGYGLQQVLGIFGVDAHRVISNHVAEVAKVLGIEAARLVIVTEIKKSMDAYGIDIDGRYMKLLGDVMTFRGEVVGINRFGIQKMRASSLMLASFEETNEHLFQAAVHGRRDPIKGVSECIIMGKHIALGTGAFDLLYRE

SEQ ID NO: 34 - protein, 681 aa, babesia duncani
MANGGLYVFPLLLCMHSVYALQCPHAHRACFLKYNGIYTSKANKLGVLDVVNVEDNALDLEDVVPLQEVYKFFNNYLMPFYHKRVSQNYGTNVPIIISCSGGVDSMALLHTFGLIKENSHTFIKKHPINYIDGSNAVIAETASNVLKYIFENVNVIYFDHKVRSDTKVDIEIIENACKKYNFNFNVQELDCNDSYFSNLEGGFQANSRKWRRRELKKYVMELHSSKMLSNEGKNKIGIRNGSYNSITTNKQDGLNSNSIGIVFMGHHANDNIETFFMKFIRGTHLLQMSEINDQSFLDSKDRIPLIRPFIHLPKNALHQFMQDFRFQYNEDSTNVLFDYGRNQFRKLVMPNLIEYIMSINKCQNDKAIDLLDKRIHVLSKQASNFRNDIEFQIQMFKVYLKSKYGDLLPIKKKRYMNRMGEIYSDFYKRLYFNRYNTAIHSNIQELKYFHDLNIPIMDLFFVDEWLILESKLLREEVLYDFFSHHFRKPLFYNAFTKIVDRLEVNFSEGSIKQYCLSKDVSMSHQGSLLKVKHRPEKNEKVLIFKDDLCSFSVLDNLQLFVEKAEGYTRNVFHLLFKVPMYATTESIDFDIRLFRNDDVLPSKILWNREAGSLLTHMKIKHIIKDYIPVIAMAGTNKIVGFYGFNIIPPYHCRGRQEVTYSFVDVAHGSTEYREYSIRVTR

SEQ ID NO: 35 - protein, 1523 aa, babesia duncani
MGFFIQSQLLSTLWILIVIPQNVQCLRNNSRNNSLAFASGYAHSNVRNSLDSLNGLPNLINRKEPTCQDGFKLFGVPRMYGWMIENLGKINQSFDTCDFYEDVDYFYIDMNAVIHSATHGNMSPIVEIEDEQRMRRITSTLLKLFHMIKPKKVMYLAVDGVCPSAKINQQRTRRFRLAKKVEDLTARVQDICEMKPIEDYNIDKLPCGSYDNVTFNPNYISPGTEFMQMFDSEIKNWLAIKTLEKQWGDCLVIYDGANIPGEGEQKIYEFMRKLNESKVKSRNKNHLVYGLDADIMMLSLLTKMPNVCVLREKRDYTPHILSKIKPEPYFTPETGIVHYHANDYIDLEAKHFDVVSMMDIRRALYNRCIKYVGLKYDLANIPFLNQENVPNRLADDFVLLSFLAGNDFLPHLPTVDLEFHTFSDMLNSYFFMLPRLHGFITRGYRIHMGRLQKLFRVLQRQEVHVFKEKAQHESVPEYRDVTKYAEHYYRVKCNINYKNRNQVTRMCQEYIKGLVWNLYYYYKGCPSWNWCYKYHYAPLVSDLSQTSGVFVSFKRGRPIKPLEHLLAVSPPNGNELLPEQYRKLSASPNGELAEFFPTDYEICEDGKVNEWEHVVKLPFLNTNKLVTHAEQANQHLKYNSLSKNKLGRVNVYKCRPHNVNKSGDNRLIFDDLTKKGLIVRDGMHYGATFSVYEERPGLAHSSCLIFARHGQTPIMIKDLVRWTRISQAANKRVLIITILYKTMNTSGWSSDFSSLSNSDKITGKDRINEQESISVPSGLHCGPAGRRTLIKWIEENYRVRKGMDDTNPGVVACRLWQDSQGFSDPVSKAAIDLLRLQGIPANVTYKTLFEQHLSKMLSSLINQSRNSQARLYSIAEKMIGMFQVAEIQPLICDVLDQLDEIPQFALPKLLDDAASANNFYKICNDGLKRKIWATSAVRLYEEMLPLFQEAVLCIQLGVLEPRMHHASFIQSCRERYEIVIEQICQMIGDCKSQASRQVFRLTMRIIKVLYVKSILGDKGDFQIYFTPHDHLDTALKLNWRDLAARSVGICFKSLQLEELDEAHDSFSSTDHSSFCALAHSIISRHQDMHKITSSDMQDLESFYTLWSLVDTVIASPCMSLDNEQIASVLNVVLGDLRIRDEIDLFDASFVLGHFEFLIKISKYIVTRSININEQLPEDQDTLGQWFSLASLGLSCGYIGACQFLFRAQWNNDTCIWHFPTETEPGKPISLSKLLGYTFPYNRSDQMCLDTLPPFFYKIVHGQASPHLSETSPVRNPYEANKASLRNLLCLYLPMVDVWKFKRAMMDLKAFEQNGYGTLLNSFKRQTFQTEKLLFYMQDMAIRDFESDDYDWTIARKLYQNLERLQALKLATPPHGTVMDPKSPQPHVDDGKRLMGTILIQGDITCGFIRMMGVSMVEASDEMARRLENYWKNLLQPYMCCMQFRLLRMLFSNWTKISSFSLDVLCKMVKGYPIVAFLPLFTKAPNCDISTALGRLSASLQGTLPNHSSKGWHRIVFTMYNKHS

SEQ ID NO: 36 - protein, 825 aa, babesia duncani
MKLQINSFNHIFFIKQNIRTNLFKLSQKPHPFNLKFFRQYSHHEIFTSIKLASNKSEMASVTQILLNRSNYKGVVTLDNSTVVSANDIVLYSEFNSKISFSLTLDTLKKLTKLQTPCKVVWNYVIDSILSDSSHFTPNEIAKILVYILNIKFNDIFIKSKIELASETLIEKLNGSLSLMVGFSVIDIKRAGLRNISDTIRHDAEFDKLDSMDFEIIWKLWYTLSKQRGNERIRGLLFEYLERGKDKVYKFDLPKVIRSMDIILSNSKSLDLLELKFLEKLFKRFLNLYNSDNIPTFGGSSNIYMTTIQAKKALYILGNICKRNPKIFENIVKHECAMLSLVNFLKQVKEITSRSLEVHCRLYNHLKNINVENTNMKHNVRIEAIKRSSNVHAKMFVYCVEFLNNFDLITKANYNNASSYSKATSDNKRSTDLDHTIFDICTNLLRCTAQSSFLIHNVTMYCRSIYQLLVLALYNTCTGINESFVKAALSSISNIQATTLQMDRDNWDISSVIRNLSYIDKGISVLADISYSSIILEPIQNANAEIVSKCSSKYEEVLMGGEILQLDKLVYCTLLNKYNIIDGTMLDVLLQFIKANELTSRELCMFCDLLLKTREMLMDDCDKRNNVLAKLLPLALELLKSIDFDFNKRTLKDLLLILECLGAFGFSCADDLSKRILEHGAKLTISQLMKLYSIVNDDYKRRIRLLLPGILKQRGKINITAILKVLNVLDIDYNLLETLEHNLGDDLYLVDKAYFARKRLLHQRNSSNPRKKTKVDYNNSGSHHMFIIDSSVAKAFIKCNKGTHSSSKLYKILVSKYQSSSSSMAL

SEQ ID NO: 37 - protein, 374 aa, babesia duncani
MQGGWLLVCIVGFLACFGASTKPNDKKATNEELTCPAVNDLQGTPIDLQYTFDRLDMTSLSRLLAIQNMYSKRNPRNIRIPKFWNTDVAHGIWSRFRIYGTNLVYKTSRGDGSCLFWSVSDSLRLGGFTIKKIKENLDHYGTKNRGIYEAIMSLPNDDEYLTMQDIQRIATIGFVGFDPDVEASVKQWDREPILSHLETVKSLKTAYMGDVIPFDLGCHRFTSFRAVDDFYNGIANDATCIKAGRDLFNHTIKYLASGAWGYEIDIYAIETALNLKIVLISYNTGSLVCYYWSEDYVPQSMILLYYHDSRHFDVAGLVDMFKVPNVRNPRAKVLTSFHIREMPMALAIILKDDCRITKRNERFSFGNEDLTHLY

SEQ ID NO: 38 - protein, 654 aa, babesia duncani
MQMFNRFLKASVALLAVASFGIQYIFAKGSNSGKIEGPIIGIDLGTTYSCVGIYKNGRVEIIANEMGNRITPSYVSFVEGTQKVGEAAKSEATINTESTVFDVKRLIGRKFTDRDVQEDMKLLPYKIINKSTRPYISLHDGKEQRTFAPEEISAMVLKKMKQVAESYLGKEVKKAIITVPAYFNDSQRQSTKDAGAIAGLDVVRIINEPTAAAIAYGLDKANAESNILVYDLGGGTFDVSVLTLDSGVFEVIATGGDTHLGGEDFDRRVMDHFIDIFKKKHKVNIRDNKQSLQKLRKEVEAAKRTLSSTTEVLVEVENLINGIDFSEKLTRAKFESLNAELFEKTLATVKKVVEDADIPIRDINQVVLVGGSTRIPRIREMIKEYFGKEPDYGINPDEAVAFGAAMQGGILSGESSDNLLLLDVCPLSLGIETLGEVMSVIIPRNTMIPAHKSQVFSTSVDNQPMVTIKVYQGERKLTKDNVILGKFDLSGIPPAPRGVPQIEVTFDIDTNGILSVSAEEKGSGNKHNIVITPDKGRLSPEEIERMIKDAEMNAEKDKEVFNRVQARQALEGYIDSMTKTINDDKTGKKLEDDEKEKIRDALDEGTKWLASNPEVGADEISAKQHEIEAICNPIISKLYGSGEDSDDSGYSDEL

SEQ ID NO: 39 - protein, 400 aa, babesia duncani
MAIPDNNNNTQSNGFDTLESNYDEVVDSFEALKLNEDLLRGIYSYGFERPSAIQQRGIKPIIENYDTIGQAQSGTGKTATFSIAALQIINYNIMSCQTLILAPTRELAQQIQKVVLALGDYLKVQCHACVGGTVVRDDIHKLKAGVHMVVGTPGRVYDMIDKKALLTDKIRLFILDEADEMLSRGFKGQIHEVFKRMPPDVQVALFSATMPNEILELTTKFMRSPKLILVKKDELTLEGIKQFFVMIDKEDYKFDTLCDLYESVTITQAIIYCNTRRKVDMLTNKMQEKDFTVSSMHGDMGQKERDLIMREFRSGSTRVLITTDLLARGIDVQQVSLVINYDLPMSPDNYIHRIGRSGRFGRKGVAINFLTPLDMDAMKSIENYYNTQIEEMPADIAAYM

SEQ ID NO: 40 - protein, 108 aa, babesia duncani
MSKKLKTKGPENINHSLQLVMKSGKVCLGFKSTRAALRSGKAWMIILSNNIPALRRSEIEYYAMLAKCSVYRYSGDNNDLGTACGKYFRVGCMAVLDAGDSDILRNIE

SEQ ID NO: 41 - protein, 418 aa, babesia duncani
MTSVNSDVDISEVSQMSDADIRVRINLIDSEIKILRSEHTRLKSRQKTLQDRIKDNLEKIQLNKQLPYLVANVVELLDFTSDDEQDDGLTPSPSQKKSKSLVIKTSTRQTIFLPVIGLIPASELHPGELVGVNKDSYLVLDKLPPEYDNRVKAMEVCEKPIEDYSDVGGLDKQIQELVEAIVLPITHQERFKKIGIKPPKGVLMHGPPGTGKTLLARACAAQTKATFLKLAGPQLVQMFIGDGAKMVRDAFSLAKEKAPTIIFIDEIDAIGTKRFDSELSGDREVQRTMLELLNQLDGFASDDRVKVIAATNRPDTLDPALLRSGRLDRKIELPHPNEQARCHILQIHSRRMNVNPDVNFKELARSTDDFNGAQLKAVCIEAGMVALRRDASELEHEDFVEGISMVQAKKKNTLNYLN

SEQ ID NO: 42 - protein, 524 aa, babesia duncani
MAGHAALILNFGSCFSGVLVRVLRDVGINCVLESAEKALDALNTNSTVKVAILCGGLDSVYDETSLTVPEEFIKACEEKNVKILAISHAFYALCKTLGARLMNGKGNDYIIDTVTVQRPMVLFNNVGKHFKAKINPINGVEVLPAGFESLATFGNGHYAAIGDEKRGIFGVAFHPESDDTENGLVILKNFCLEQGACPIEWSMEQYLKDELARCIAQCGDTKVVVAGLSGGVDSTVCAAIVHKAIGNRFHGVMINTGLMRLDETKKCAERLKKEIPGIQLHIRESADVFFGELKGILDPEQKRKIIGKVYIDEFERAIKDLGFDKSNCLLLQGTIYPDILESELNRRNQMPIKSHHNVGGLPKDLALELIEPVRLLFKEEVRKLGRLLGLSQESCERQPFPGPGLGVRVIGELNPRNLDLVRRADAVMNQILDARGYRSKISQSGCILLADVHNTGIRNSGRTYGHAVIIRIIITTDFVTAQWARIIDTDCLAEISKTITDTVPEITRVCYDITDKPPACIEWE

SEQ ID NO: 43 - protein, 211 aa, babesia duncani
MAEEMPQFKLLLVGDGGVGKTTLVKRHLTGEFEKKYIPTLGVEVHPLKFRTNCGGIQFNAWDTAGQEKYGGLRDGYYIKGECAIIMFDVTSRITYRNVPNWHRDIVRVCENIPMVLVGNKADVKERQVKAGHIQFHRKRNLQYYDLSARSNFNFERPFLWLSRRLLNQPQLVFVGECAKAPEIQIDPLLAQQSERDLEAAARVAIDDDGDL

SEQ ID NO: 44 - protein, 612 aa, babesia duncani
MADRFTGRNNREAVVAYPGWFSETQKQCLRACVTASGLSCLRVISHVHAMAMDYGVYRVKQLNDETPTRVALVMIGHCHASAAIVDFYASHCSILSQVSRRNLGGRNLDMMLMKYMATEFSKKYHCDPLENNKTRLKVEAVAVKTRRVLSANAESSYSAECLMEDNDMSGHITRTQFEEMCNAEFIPQLIEMLKECIEASRTDLDSIFSVEIAGGSSRIPCIQQAISSIFNKVPSRTLNADECIARGCVLEAAIKSNHYRVREYKTRLTLPRSLTLGYFNGQEPMLLEAIAAGTPLGDPIRVTLQAQAPVCVRVALGDALDPRSQDALGTLDIARHISQEAQPAPVTTNDGAAIQTDEQDAEIQSESSPSGGISVTLGFDDCGQFVASPECCEYRWLPATILDIARLEAAELEARGRDLKENSRLQALNDFETLLYTVRDKMQSSHRDFIDPQMIPAYESELDHWREWLYENSGASQETLQEGIDKVSSEWKRIDKYFKEHQNKLENLEPFLQRLQERYNFCCEDNNPNWHGATPEERLNFAQELMDLDSRVRQMHQDESQRPRHMEPLFTMQQIQGEMQKLLVSISEFCQAKAAKAPAQEPPEQQPKEQQE

SEQ ID NO: 45 - protein, 152 aa, babesia duncani
MDAGGKIGGKIGGKVGGMGKGGKGKTGSGKGKKAPMSRAARAGLQFPVGRVHRMLKSRISADGRVGSTAAVYASAILEYLTAEVLELAGNASKDLKVKRITPRHLQLAIRGDEELDTLIKATIAGGGVIPHIHKALMNKGPAQVLVKPPKRI

SEQ ID NO: 46 - protein, 396 aa, babesia duncani
MGIVTASIAPLHIGDDLYTRMKTLEKKLEICEIQENYVREEYRNLKLELIRAREEIKRIQSVPLVIGQFLDMIDKNYGIVSSTAGSNYYVRILSTINRELLTPNSSVALHRHSHSVVDLLPPEADSSIQLMQVSEKPDVTYADIGGLDIQKQEIREAVELPLTCPELYRQIGIDPPVGVLLYGPPGTGKTMLAKAVANQTDAKFIRVVGSEFVQKYLGEGPRMVRDIFRLARENAPAILFIDEVDAIATKRFDAQTGADREVQRILLELLNQMDGFDQNATVKVIMATNRADTLDPALLRPGRLDRKIEFPLPDRRQRRLIFQTITSNMNLAADVDLETFVARPEKVSAADIAAICQEAGIQAIRKNRYVVTTKDFERGWKRHIRKHERDYGFYGV

SEQ ID NO: 47 - protein, 712 aa, babesia microti
MSSQETFEFNADISQLLSLIINAFYSNKEIFLRELISNASDALEKIRYELLRDGTKVSDESEFSIKISADKSNNTLTIEDSGIGMTKADLINNLGTIAKSGTKAFMEAMQSGCDMSMIGQFGVGFYSAYLVAEKVTVVSKHNSDEQYIWESSASGVFTITKDETTEKMKRGTRLILQLKEDQTEYLEERRLKELVKKHSEFISFPIHLLCEKTKEEEVTASDDEGDKKEDDKKEDDEKEDDKKGEDEKVEDVSEDKKKTKKVSTVTKEWEVLNKQKPIWMRQPNEVTNEEYANFYKNLTNDWEDHLAVKHFSVEGQLEFRAILFIPKRAPFDMFENRKKKNNIKLYVRRVFIMDDCEELIPEWLSFVKGVVDSEDLPLNISRETLQQNKILKVIRKNLVKKCLELFSELTEKKDDFKKFYEQFNKNLKLGIHEDSANRNKISELLRFETTKSGDEAISLREYVDRMKPNQKYIYYITGESIQAVSNAPFLEKLKDKNIEVIYMTDPIDEYAVQQIKEFDGKKLRCCTKEGLDIDDEKDEEEEKRFEQVKQEMEPLCKTIKEVLHDKVEKVTCGKRFTTSPLALVTSEFGWSANMERIMRAQALRNSSITSYMVSKKTMEINPYHSIMKALKERVAADKSDKTVKDLIWLLYESALLISGFNLEEPTQFGNRIFRMIKLGLALEDDQPDDTDLPPLDEGVAVDGGDSKMEEVD

SEQ ID NO: 48 - protein, 447 aa, babesia microti
MPKEKTHINLVVIGHVDSGKSTTTGHLIYKLGGIDKRTIEKFEKDSSEMGKSSFKYAWVLDKLKSERERGITIDITLWKFETQKYEYTVIDAPGHRDFIKNMITGTSQADVAMLVVPAESGGFEAAFSKEGQTREHALLAFTLGVKQMIVAINKMDSCQYKEDRYMEIFKEVQQYLKKVGYKVESVPFVAISGFHGDNMVEKSTNMPWYKGKTLVEALDQMEPPKRPVEKPLRLPLQSVYKIGGIGTVPVGRVETGQLKAGMIITFAPTGLTTECKSVEMHHEVVEVASPGDNVGFNVKNVSVKDIKRGNVASDSKNDPAKEATSFSAQVIVLNHPGTIKAGYSPVVDCHTAHIACKFESLDTRIDKRTGKTLEENPKTIKNGDAAMVTMKPNKPMVVETFTDYAPLGRFAVRDMRQTVAVGIIKAVEKKDPSSAKVTKSAVKAGKK

SEQ ID NO: 49 - protein, 438 aa, babesia microti
MTKIISACGREVLDSRGNPTVECEVTTEGGKFRAIVPSGASTGIYEALELRDGDKTRYLGKGVQNAIKNMHNIICPGIQGFLCTEQEKLDNHMVKVLDGTQNEWGFSKSKLGANTILAVSMGAARAGAAAKGIPLYEHLAQLSGKPTDKFIMPVPCLNVINGGSHAGNALAFQEFMILPTVADNFSNALRMGVEVYHTLKKVINKKYGQDATNVGDEGGFAPNISTPQEALDLLVEAIAAAGYTGKIKIAMDVAASEFYQKDVKMYNLTFKSSSPDIKTSDQLVELYKELVNKYPIVSIEDPFDQDDWEAYAKLTAAIGDKIQIVGDDLLVTNPKRIEAAIQKKACNALLLKVNQIGSVTESIQACKISQENGWGVMVSHRSGETEDVFISDLVVALGTGQIKTGAPCRSERNAKYNQLLRIEQELGERATYSKVFNK

SEQ ID NO: 50 - protein, 77 aa, babesia microti
MQIFVKTLTGKTITLEVEPSDTIENVKAKIQDKEGIPPDQQRLIFAGKQLEDGRTLSDYNIQKESTLHLVLRLRGGD

SEQ ID NO: 51 - protein, 282 aa, babesia microti
MQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGVIDPSLALLAQKYNCNKMVCRKCYARLPPRATNCRKKRCGHCNDLRPKKKIKGN

SEQ ID NO: 52 - protein, 376 aa, babesia microti
MGDDDNAALVVDNGSGNVKAGIAGDDAPRCVFPSIVGRPKNPALMIGMDEKEVYVGDEAQSKRGILTLKYPIEHGIVTNWDDMEKIWHHTFYNELRVNPEEHSVLLTEAPLNPKTNREKMATIMFETHNVPAMYVAIQAVLSLYSSGRTTGIVLDSGDGVSHTVPIYEGYAMPSAIMRLDLAGRDLTEYMQKILVERGFSFTTSAEKEIVRDIKEKLCYIALDFDEEMQAAETSSDLEKSYELPDGNIITVGNERFRCAEVLFQPSFIGKECHGLHKTTFDSIIKCDVDIRRDLYSNVVLSGGTTMLQGIGERLTKELSCLAPSTMKIKVVAPPERKYSVWIGGSILSSLSTFQQMWITKDEFDESGPVIVHRKCF

SEQ ID NO: 53 - protein, 187 aa, babesia microti
MHNHSYYTILLTIALIHTTGCHGFLHRNIKFILHSMTVYGKPKRLHKTPPWAHLFEEKVEPSPLGEPWAKLSKDVANGERRVRLTVKKSHLQIYAAVVDDYKNQVICIASSNLPVLADVLGTVPTKDPTVRRNKGNNVKAAYEVGKHIGRLALAKGVAKVYFDRAGYKYHGRVEAVAIGARKVGLQL

SEQ ID NO: 54 - protein, 425 aa, babesia microti
MYQIDRMIDKHKDPIDPLQTILSVKGTMKCKLSEMLTRHSTEDRHSLSLVADLKRESPTHTDSRSGVRLSFLDAGEVVVTMANTGFDVVLVNTDDIAYKGTLDDLKTSICAAHAIGNRSRPAVVMKDIILHPLQLAQAAQLHADGVVLNSFYLGPALESMIDTSYNLGIEPIVEVHTLEDALYAIQLHTKILMINQWDRLTNKCHPNRALQIREIVPDGIITIACGGIKTLEQIEQLGLAGYDAVVLGKKLADTNIPSFVGSIKKWNAPGKGILAISKPLFFTDEMDEKDVSGGGHVIRMKQNYRQTLQEMCEFYQIAENNCVEAPRPKDVLLRFIGNDMPEKWIFKREKWMKDHAHEYPSEDHATAVYDFNLAVELNTTLECNKKLLSQHFKREMVDKLEEAIKNYVKKCYINLKRFTNKISCS

SEQ ID NO: 55 - protein, 644 aa, babesia microti
MSQGPAIGIDLGTTYSCVGVWKNETVEIIANDQGNRTTPSYVAFTDVERLVGDAAKNQDARNPENTVFDAKRLIGRKINDPCIQSDIKHWPFTVAAGPNDKPVIKVQFQGETKSFHPEEISSMVLTKMKEIAESYLGKTISNAVITVPAYFNDSQRQATKDAGTIAGLNVMRIINEPTAAAIAYGMDKKGTSEKNVLIFDLGGGTFDVSILTIEDGIFEVKATQGDTHLGGEDFDNRLVNFCVDDFKRKNGGKNISTNRRALRRLRTQCERAKRTLSHSTQATIVVEAIFDGIDYSCNITRARFEELCAEMFKNTLIPVEKALADADMDKKQIHEVVLVGGSTRIPKIQQLIKDFFNGKEPCKSINPDEAVAYGAAVQAAILTGEQSSKVQDLLLLDVTPLSLGLETAGGVMTVLIPRNTTIPAKKEQEFTTNENNQTGVMIQVFEGERSMTCDNNLLGKFHLTGIPPAPRGVPQIKVTFDIDANGILTVSAADKSTGKTEHVTITNDKGRLSQQDIDRMVAEAEKFREDDEKKKRCVESKNELENYCYSMKNALEEEGVKSKLSSSELSEAQKLLQNTFSWIESNQLAEKEEFEAKLKEVQAVCTPLTAKLYQAGGGVPGGAAPGGFNAGGAAPSGPTVEEVD

SEQ ID NO: 56 - protein, 395 aa, babesia microti
MTAYIPGLLERLSAPIVSSRLDDDDLAYLGKYESEISDSDNLQNIYSNDTIVALGDLDAKYKQQTYLSSLNSHIVDQDAQIAFYPVKYTNKIIRNDSNLTYRRTTSSLSNPLVNYIQKSQHDTPDPVDSNIQNSSSEQYAITTASSGSDKSKRLSHDNSKTDHDIHDKSGVVTANNHSTDRFQNAGDLVWQPGFTRQLGTKGRRVLLDLIRKVYRGNPEYFKNILALKNPPTSISNLPFCNVTMLWELANDFGVFDQAIQIHYAHGKPGYPANYHNTANYGCNKTRKTNSGKSKKNKFYNYDYEAYDEYYEDEYISSFSNGRTSRGRKIVQPKRFDDYDHLLDSNYQVEYGYCSNRHLNKDGNTILYYKNKSHDYVNLGELKGHKIERFTSQLIC

SEQ ID NO: 57 - protein, 404 aa, babesia microti
MKNDFNSVELPGHFLFTSESVNEGHPDKLCDQISDSILDACLEQDPESKVACEVCTKRGMVMVFGEISTNAKVNYEEVVRNVVKNVGYDSEDKGIDYKTMDVIINLDQQSHEIAQAVHLGKDADNTCAGDQGIMFGYATDETPEYMPLSHSLATGLGKRLKDVRLSGLLPYLGPDGKTQITVEYIKEGYGSIKPIRVHTVLISQQHSANVSNDKLREDLMTHVVKAVIPPHFLDDKTKYYLNPSGHFVVGGPSSDAGLTGRKVIVDTYGGWGAHGGGCFSGKDGTKVDRSAAYYARKVAKSLVANGFCRRALVQVSYSIGIRSPLSLHVDSYNTCIEGFTDLDLEQIAVRNFDFSVGNIIKELQLKKPIYSQTGVYGHFGKDNPEYLWENVKDLSHELTHKPKR

SEQ ID NO: 58 - protein, 467 aa, babesia microti
MDYTRSLFTLSGPATASEVEKHIQNAIEFVKRRDPDQVQFIQAFTEVANGLAPVFQTDLKYLEIFLSLSEPERVITFKVPWVNDAGKLMINRGFRVQFNSTLGPYKGGLRFHPSVNLSILKFLGFEQIFKNSLTTLAMGGGKGGSDFDPKGKSDNEVRSFCQSFMTELQRHIGPDTDVPAGDIGVGEREIGFMYGQYKRLSNSSTGTLTGKDPKWGGSFIRPQATGYGLVFFVQYILNDLHNGDSFKGKRVAISGSGNVAQYAADKVIDFGGIPITFSDSSGYIYEPNGFTKEMVTVLMELKNIQRARVSEFLKYSNTAKFFPNKKAWDVDTNVNVALPCACENELDKADAEMLVKKGCIIVGEGANMPTTPEAISVFKAAKVTVCPGKAANAGGVAVSGLEMSQNSQREKWTSEKVLEKLQDIMKNMSKACQEAAAKYNVHGDIISGANIAGFLKVAHSYCDQGCV

SEQ ID NO: 59 - protein, 433 aa, babesia microti
MSYSAEETDASIEQWKILRLIRNLESAKGNGTSMISLIIKPKDEIARINKMLADEFGTASNIKSRVNRLSVLSAITSTQQKLKLYRQTPPKGLVVYCGTILTEDGKEKKVSLDFEPFKPINTSLYLCDNKFHVEALKELLESDEKFGFIIVDGNGVLYGTLQGNTKEVLHSFTVDLPKKHGRGGQSALRFARLRLEKRHNYVRKVADIAVQMFITNDRPNVSGLVLAGSADFKNDLMSSDMFDPRLAAKVVKIVDVSYGGDHGFNQAIELSAQCLSNVKFIQEKKIISRFFDELAHDTGRYVYGVHDTINALEMGAVEMLIVYEALDIQRLQMRNPVTGEESVIIQTSERDTEAMRDPVNNVDLELVESIHLSEWLVNNYRNYGATLEFITNKSQEGSQFHRGFGGIGGILRYKLDMSEYDLPVNDNDFDDFI

SEQ ID NO: 60 - protein, 1094 aa, babesia microti
MDSLVPPYNKLNFDISRPDISGNTLHYTFFQYPDSSISKLRNVFAHNPQQNFTNQPFYPPKHNDTPHTEQNGSQFIHSNSNTSNNLESNDVDNNASASACDKRSFPHDDRYSSSNYNEYPGILSSIQDIADLFDLDNYHIYHGIDNISYLTYSTDANRTGIDSKLEFIYEVLNSNGSNMTLSKLEGFLRILTTDDNPIGTLTPYGSLMAHTCLRFLKSYIRTDNKGNSHDPDTFIYSIEGKKGKSKLSKQGITNENETTTVSSDFRRIAMFNFELYSKQLMDTIHSQDKSIAINKRKFDDLTSNYTKSESVSIDSMSAISDSTDKRAVSSQTLKIIKQGSAYLQDFISKYRIGFEPHFPRIVCGSIDRSLVTNQASITVPIDKSGRSVHIAFEALSDSYGMSVWPELNQLPNSLQLGTKIQIAWHKTPGNMRWYIGTIVQSTASQYSVQVYLGKNEYKTRYCTMKYFVPPNVPVVKPDNTWWRLVPREINLSTDLYVGSCVSLYDIISNFESIDCVICNVYYSNDNSPNGPIKGPNEEIYKSDKSSSGFKIQLNCACHGPTRRIRQGVLTTGVVRVHIYCMSHKIDKIIPIDSIRGYKLNYDLHAHFDGQPDYGPLMQSTTWTWVIPRLVIAPDNKYDDEKYWTSNPKYSLLIFPYHRNMEYIISATKLIAILHPELNVTSLYNNGEWFVRQEGDIAIKGYGGLRPSGLSFSKIPLLDRLFGCHRIKVFYNVPIDVNKKVEDFKNDYTISRCANCRGIYYTDISHASNGKTYISSDVEYRKKIQIAAAIRWKRVRESSNTILNCYNENDELVYRTKNSFSLSWEQDGRNLELAYRRAHAPGISKFESKRLDSLMKSNKQVSNVPENMCTEGLLEKFNESMKFSESVKSTDSKNAAKLLEVAILGGWMNDSTKTESINTNKGDNIFRYINKSIQAKAAQDALDFLKNCSPPCLSKQVGKHYTPINGKQSMFKQGGMSDYQDSIPHLDIGNPMISDYNSKKVKLQSVSGDQRGLNIYENPCIATVHDPSVNYNSLRNFLAKAEWDEEIEDAPSHGVLDFTIKSEEPVHNEAGEDCIYAVPINDSVNIIWSDTEDAK

SEQ ID NO: 61 - protein, 1024 aa, babesia microti
MVERLIARSLSIDSIETQILRFFSGDIGLQTVLLEFQKGPRAFVICLDIINKHCKEFSSKSLPLILFCSQTLVECDHIYSLCRLVSDGQAQDLDKSDATKLDTEREETIEKLERVINLLEQLNKVNPQIFPSLRFLLCAWLRLKLFDNWSKGITFEIIQFVGTFQSNRQFQIELLAILAQEICNDKFILSIARRNELAKNCISQAPMVFKFLGVKDSSIGSVYSDWIQLHVKYLDSIVDEEQCELPSDVDLMVKLSLSTLISLNLIDKLFEQSPLPIHTITNLIVLCPLDNHTALQDDCDMINPLVNEILTLIICNLSKLYKIQSPLFKTTSPILLSLSYEQLPYLIEHQFSEDIDNILDGTIRILSNGDYPTRDVMVNFWINLKRNINTQNGWEKLADYLQKIVIVFYEMPLYETEKYDFAELCSFRESASMLLFEIADAIGHQFIFDIVECRLQVLVETINLKEQITVSFSEIESIFFILSAISSNAEMGKDTCIPTALALLNKLKYPTQGSLSLLLSISIGRLILWTAEYSGKKTDLFNCLFMLIIGTLLPSILRIKNNSASKGMYYYLLGENILIDAMLALSRTSRKIVSEDTQEVKKYLICIYQIIKNVQFSIENRIKAVNAAGAIISYMPIAEMKTLFSDLIENLSNNIKSVSNPTDYIQLYLMAMQSVCPIPDCIESEVAILKIVEKHVSVMELLFTSSNEDIIERLSQVLVVVNRISRNHSEASPLFLWTLKLLSVSFRCSHPSHPYSALRSILINVNDCSVENWASISNSLLPSLAMLKDSIVSCCVGDKDRNLLQQQMSDLTMDFNTSQLLSAPDSVGLCVDCLNVALNRHEIANFLLDKSKFIIFLESLLIILPYIVHPKVLHACMILIKLVASIDMNEASGNDYSDSAVIGTSVYAKKIRLLNIISQRCNESSDEIHTITFKIVTAILSTVISGTCGVETWIDVAAETILALMCDGKTADEAKNAIDSFFEMISEPSINVTIKNDYCNKLKVPNTLFQALLQLEGWKLWKKC

SEQ ID NO: 62 - protein, 438 aa, babesia microti
MRGMFSNKWMSFVCFSILFVALKSDLEYVSALKLLRAPPQTSLFLEKLIDDGSDIPKDPIDTDKEESQSSLFKFNLNLFNKKSIWEADEKFVITLAKSRLNVILAQKLDKFLAKTCKIYTVDSEHSACINDIKIYAQKCIESNDLNSCYVIPIQPIAKLPTSRLYGLVPHVLNFSILIFTNLRSNLDRYYIDGSKDWFSHIFMRLKRFFGIRNKHSYFSDNRLMNKIFSRTSTTFGPDRSDSLLSNYIKFGAIEYAILLNTRSNLVKMILSSFAHIKFVRKRLYKFYTNKWKSIEGLVTRGHLKPVDLSNNPISDNIFKYFGKFSNNTNLSNAIAGAFLDHYKSLFSNSTDVNGEGSSGEGPSGEGFNGEGSSGEGPSGEGFNGEGFDGEGPSGEGPSGEGFNGEGFNGEGLNGEGPSGEGPSGEGLNEWNGLMNGTA